(19) United States
(12) Patent Application Publication
(10) Pub. No.: US 2025/0207105 A1
(43) Pub. Date: Jun. 26, 2025

(54) FROZEN FORMULATION COMPRISING APOPTOTIC CELLS

(71) Applicant: ENLIVEX THERAPEUTICS R&D LTD, Nes-Ziona (IL)

(72) Inventors: Shai NOVIK, Ramat Hasharon (IL); Oren HERSHKOVITZ, Kibbutz Revadim (IL); Veronique BAROUKH, Rehovot (IL); Chen ANKRI, Ness Ziona (IL)

(73) Assignee: ENLIVEX THERAPEUTICS R&D LTD, Nes-Ziona (IL)

(21) Appl. No.: 18/852,502

(22) PCT Filed: Apr. 18, 2023
(86) PCT No.: PCT/IL2023/050402

§ 371 Date: Sep. 30, 2024

Related U.S. Application Data
(60) Provisional application No. 63/332,286, filed on Apr. 19, 2022.

(51) Int. Cl. C12N 5/00; A61K 35/17

(52) U.S. Cl. CPC C12N 5/525; A61K 35/17

(57) ABSTRACT

Disclosed herein are frozen formulations of apoptotic mononuclear enriched cell populations, methods of making the frozen formulations, and methods of using the frozen formulations following thawing of formulations. Frozen formulations further include an isotonic crystalloid solution and a cryoprotectant. The thawed formulations of apoptotic mononuclear enriched cells maintain the biological activity observed in fresh cells prior to freezing.

### FIELD OF INTEREST

Disclosed are frozen formulations of apoptotic cells and methods for preparing frozen formulations of apoptotic cells. Apoptotic cell populations may be maintained and stored as frozen formulations, and then thawed for use to treat a subject in need.

### BACKGROUND

Cryopreservation is a process for preserving cells by cooling to sub-zero temperatures. At these cold temperatures, any biological activity, including the biochemical reactions that would cause cell death, is effectively stopped. However, cryopreservation is known to cause damage to frozen cells and thus affects cell survival. The cell survivability during the cryopreservation is measured by cell recovery and viability after thaw. Cell survival during the cryopreservation can be significantly affected by process variables such as e.g., cryopreservation formulation, freezing cycle and procedure, and thawing procedure. Phenomenon causing damage that occur during freezing include but are not limited to solution effects, extracellular ice formation, dehydration, and intracellular ice formation. In addition, it is now clear that low recovery of cryopreserved cell populations is largely caused by apoptosis.

Apoptotic cells present one pathway of physiological cell death, most commonly occurring via apoptosis, which elicits a series of molecular homeostatic mechanisms comprising recognition, an immune response and a removal process. Interestingly, apoptotic cells are immunomodulatory cells capable of directly and indirectly inducing immune tolerance to dendritic cells and macrophages. Apoptotic cells have been shown to modulate dendritic cells and macrophages and to render them tolerogenic and inhibit proinflammatory activities such as secretion of proinflammatory cytokines and expression of costimulatory molecules.

Populations of apoptotic cells may be produced for therapeutic purposes. As summarized recently (Trahtemberg and Mevorach (2017) Apoptotic Cells Induced Signaling for Immune Homeostasis in Macrophages and Dendritic Cells. Frontiers in Immunology, vol. 8, 1356) apoptotic cells may have a beneficial effect on aberrant immune response, with downregulation of both anti- and pro-inflammatory cytokines derived from PAMPs and DAMPs, in both animal and in vitro models. Further, administration of apoptotic cells has been shown to be effective in models of cancer, independent of any other treatment. Moreover, a marked synergistic effect was observed when apoptotic cell populations were administered in combination with other cancer therapies, further delaying disease onset and progression, and improving survival (See for example, International Publication WO 2018/225072).

Thus, apoptotic cells provide a therapeutic solution for treating a range of diseases and conditions, including but not limited to cytokine storm, cytokine release syndrome, autoimmune disorders, viral infections, osteoarthritis, GVHD, sepsis, lung dysfunction including ARDs and fibrosis, COVID, and cancer.

It is important that any cryopreservation formulation developed for apoptotic cell populations, developed as a vehicle for delivery of an apoptotic cell product, be able to support and to maintain high cell survivability, preserve the product integrity during thaw, and maintain the apoptotic cell characteristics. The formulation also needs to be safe to administer to a recipient. Furthermore, the cryopreservation formulation should support product stability over a long period.

Freezing solution composition also influences cell survival; therefore, appropriate formulation is needed to enhance the stability of cell-based products; to ensure preservation of product characteristics and quality attributes and to prevent it from being damaged by harmful factors. Several commercially available serum- and proteins-free cryopreservation solutions have been developed and are widely used in cell therapy. Yet, there remains an unmet need for cryopreservation formulations and methods of use thereof with apoptotic cells.

The formulations and methods described herein, are directed to reducing possible known adverse effects caused by the high levels of dimethyl sulfoxide (DMSO) transfusions and maintaining the properties and therapeutic effectiveness of apoptotic cell populations following thaw of a frozen formulation of apoptotic cells.

### SUMMARY

In certain aspects, this application discloses frozen formulations comprising: a population of apoptotic mononuclear enriched cells; an isotonic crystalloid solution; and a cryoprotectant agent; wherein the formulation comprises a pH range of about 6.5-8.0.

In a related aspect, the concentration of said apoptotic cells is between about 17-150×10\hat{ }6 cells/ml.

In another related aspect, the mononuclear enriched cells comprise at least one cell type selected from monocytes, lymphocytes, T cells, B cells, and natural killer cells. In a further related aspect, at least one cell type comprises a single cell type selected from monocytes, lymphocytes. T cells, B cells, and natural killer cells.

In another related aspect, the isotonic crystalloid solution comprises sodium chloride, potassium chloride, magnesium chloride, sodium acetate, or sodium gluconate, or any combination thereof. In further related aspect, the isotonic crystalloid solution comprises 140 mmol/L sodium, 5 mmol/L potassium, 1.5 mmol/L magnesium, 98 mmol/L chloride, 27 mmol/L acetate, and 23 mmol/L gluconate.

In another related aspect, the cryoprotectant agent comprises DMSO. In a further related aspect, the percent DMSO in said formulation is at a concentration of about 2.5%.

In another related aspect, the pH is at about 7.4.

In certain aspects, disclosed herein is a method of preparing a frozen apoptotic cell formulation comprising steps:
resuspending a freshly made apoptotic mononuclear enriched population of cells in an isotonic crystalloid solution having a pH of about 6.5-8.0;
mixing said resuspend apoptotic cells of step (a) with a cryoprotectant agent; and
gradually freezing said mix of step (b) to a temperature of about −196° C.

In a related aspect, the concentration of said resuspended freshly prepared apoptotic cells of step (a) is between about 25-300×10\hat{ }6 cells/ml. In a further related aspect, the mononuclear enriched cells comprise at least one cell type selected from monocytes, lymphocytes, T cells, B cells, and natural killer cells. In yet a further related aspect, the at least one cell type comprises a single cell type selected from monocytes, lymphocytes, T cells, B cells, and natural killer cells.

In another related aspect, the isotonic crystalloid solution comprises sodium chloride, potassium chloride, magnesium chloride, sodium acetate, and sodium gluconate. In a further related aspect, the isotonic crystalloid solution comprises 140 mmol/L sodium, 5 mmol/L potassium, 1.5 mmol/L magnesium, 98 mmol/L chloride, 27 mmol/L acetate, and 23 mmol/L gluconate. In still a further related aspect, the isotonic crystalloid solution comprises a pH of about 7.4.

In another related aspect, the mixing of step (b) is 1:1 (vol/vol) with said cryoprotectant agent.

In yet another related aspect, the cryoprotectant agent comprises DMSO. In a further related aspect, the final concentration of DMSO is about 2.5% DMSO.

In another related aspect, the method of preparing a frozen apoptotic formulation further comprises a step of irradiation of said apoptotic cell population following said resuspension step (a) and prior to said mixing step (b).

In another related aspect, the frozen formulation prepared comprises a concentration of about 20-100×10\hat{ }6 cells/ml apoptotic cells.

In certain aspects, disclosed herein is a method of treating a subject in need with a frozen apoptotic formulation of claim 1 comprising:
thawing said frozen formulation; and
administering said thawed formulation or a portion thereof to said subject in need;
   thereby treating said subject.

In a related aspect, the method of treating further comprises a step of analyzing said thawed apoptotic formulation for Annexin V staining, wherein administration comprises administering an apoptotic population of cells comprising at least 35% Annexin V+ cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

The subject matter regarded as the frozen formulation of apoptotic cells, methods of preparation and use thereof is particularly pointed out and distinctly claimed in the concluding portion of the specification. The description of these frozen formulations and methods thereof, however, both as to organization and method of operation, together with objects, features, and advantages thereof, may best be understood by reference to the following detailed description when read with the accompanying drawings in which:

FIG. 1. Analysis of Allocetra-OTS cells following AnV and PI Staining. A frozen formulation of Allocetra-OTS produced using the methods described herein, was thawed and stained with Annexin V (AnV; X-axis) and Propidium Iodide (PI; Y-axis) to assess the prevalence and state of the apoptotic cells. The graph shows that 60% of the total thawed apoptotic cell population (comprising the mature and early apoptotic cell populations), met the criteria of ≥35% AnV. The late apoptotic population was shown to have the highest PI fluorescence intense (Y axis). There is a distinct separation between the apoptotic population and the late apoptotic population. The late apoptotic cell population comprised 22.2% of the total product cell produced and was ≤30% AnV.

FIG. 2 presents a schematic of one embodiment of an optimized cryopreservation method for apoptotic cells, as disclosed herein. The stages presented on the left-hand side of the schematic include steps in the preparation of a fresh apoptotic cell formulation, while the stages presented on the right-hand side of the schematic include steps in the preparation of a frozen apoptotic cell formulation. Italicized text provides an indication of difference between the methods.

FIG. 3 presents a schematic of the traditional main manufacturing stages of a drug product comprising apoptotic cells.

FIGS. 4A-4B present effects of CryoStor® based cryopreservation media on frozen Allocetra (apoptotic mononuclear-enriched cell population). FIG. 4A shows late apoptosis, while FIG. 4B shows apoptosis levels. The dashed gray line denotes the 7.5% CryoStor® CS based cryopreservation medium (7.5% DMSO final) formulated by centrifugation after the completion of the standard Allocetra manufacturing process. The black line denotes the formulation of the 5% CryoStor® CS based cryopreservation medium (5.0% DMSO final) created by the 1:1 dilution of the Allocetra cell suspension with the CryoStor® CS-10 commercial medium. The solid gray line denotes the formulation for the cryopreservation medium (the 2.5% CryoStor® CS based medium (2.5% DMSO final) created by the 1:1 dilution of the Allocetra cell suspension with the CryoStor® CS-5 commercial medium). The dotted line marks the Allocetra-OTS apoptosis and late apoptosis assays' release criteria wherein apoptotic cells comprise at least 35% apoptotic cells (early and mature) based on at least 35% AnV+ cells and IP^{DIM}.

FIGS. 5A-5B present the effects of home-brew cryopreservation media. Effects of home-brew cryopreservation media on frozen Allocetra (FIG. 5A) late apoptosis and (FIG. 5B) apoptosis levels are presented over time following thawing of the frozen formulation. The dashed gray line denotes the 7.5% DMSO home brew cryopreservation medium, the black line denotes the formulation of the 5% DMSO home brew cryopreservation medium, and the continuous gray line denotes the 2.5% DMSO home brew cryopreservation medium. The horizontal black dotted line marks the Allocetra-OTS apoptosis and late apoptosis assays' release criteria, wherein above 30% late-stage apoptosis is not acceptable and above 35% apoptosis (early and mature) is required.

FIGS. 6A-6D present the effect of cryopreservation media on late stage and apoptotic Allocetra population. Effects of cryopreservation media on frozen Allocetra late apoptosis levels analyzed by Attune NxT (FIG. 6A) and Calibur (FIG. 6B) FACS devices, over time from thaw. The gray line denotes the 2.5% DMSO home brew cryopreservation medium and the black line denotes the formulation of the lead cryopreservation formulation of 2.5% CS medium. The effects of cryopreservation media on frozen Allocetra apoptosis levels were also analyzed by Attune NxT (FIG. 6C) and Calibur (FIG. 6D) FACS devices. The gray line denotes the 2.5% DMSO home brew cryopreservation medium and the black line denotes the formulation of the lead cryopreservation formulation of 2.5% CS medium. The horizonal black dotted line marks the Allocetra-OTS apoptosis and late apoptosis assays' release criteria, wherein above 30% late-stage apoptosis is not acceptable and at least 35% cells in apoptosis (early and mature) is required.

FIGS. 7A-7E present the effects of excipients on the cryopreservation media. FIG. 7A shows the effects of 30 mmol/L Trehalose on apoptosis and late apoptosis levels of cryopreserved Allocetra following thaw. FIG. 7B shows the effects of 0.2% Tween-80 on apoptosis and late apoptosis levels of cryopreserved Allocetra following thaw. FIG. 7C shows the effects of 1.25% Dextran-40 and 0.63% Dextrose combination solution on apoptosis and late apoptosis levels of cryopreserved Allocetra following thaw. For FIGS. 7A-7C, black bars represent the apoptosis percentage, and the gray bars represent late apoptotic cells. FIG. 7D shows the effects of excipients addition to the cryopreservation media on cell population composition levels. The gray bar of the stack represents the CD3 lymphocytes cell levels, the white stippled background bars represent the monocyte/macrophage cell levels, the gray background stippled bars represent the granulocytes cell levels, the black bar in the stack represent the B lymphocyte levels, and the diagonal stripes bar in the stack represent the NK cells level. FIG. 7E shows the effects of excipients addition to the cryopreservation media on CD45 MNC population levels.

FIGS. 8A and 8B present the effects of Allocetra base media solution on (FIG. 8A) late apoptosis and (FIG. 8B) apoptosis levels. The black column denotes PBS base solution results, the meshed column denotes the saline base solution results, the gray column denotes the Hartman base solution results, and the diagonal stripes column denotes the PlasmaLyte base solution results. The horizontal black dotted line marks the Allocetra-OTS apoptosis and late apoptosis assays' release criteria of at least 35% AnV+ and less than 30% PI of the total cells in the population.

FIGS. 9A and 9B present the effects of cryopreservation base solution identity in home brew media on Allocetra (FIG. 9A) late apoptosis and (FIG. 9B) apoptosis levels. The black line marks the results of formulating the cells into Lactated Ringer's solution using the LOVO whereas the gray line marks the results of formulating the cells into the PlasmaLyte148 solution. The black dashed line marks the Allocetra-OTS apoptosis and late apoptosis assays' release criteria.

FIGS. 10A-10B present the effects of cryopreservation base solution identity in home brew media on Allocetra (FIG. 10A) late apoptosis and (FIG. 10B) apoptosis levels. The cells were formulated into CryoStor basal solution (CSB) using the LOVO followed by the addition of the equal volume of the CS-5 solution to a final concentration of 2.5% (denoted by continuous black line), or by 1:1 dilution with a commercial CS-10 solution and a PlasmaLyte148 equal volume mixed solution creating the 2.5% DMSO formulation (denoted by dashed gray line), or by the addition of WAK's Cryo-Sure DMSO solution at a final concentration of 2.5% (denoted by continuous black line) in comparison to the average standard frozen Allocetra drug product formulation (denoted by dashed black line). The horizontal black dotted line marks the Allocetra-OTS apoptosis and late apoptosis assays' release criteria.

FIGS. 11 and 11B show the effects of liquid nitrogen storage time on post-cryopreservation Allocetra cell population stability: (FIG. 11A) late apoptosis (AnV+, PI high+) and (FIG. 11B) apoptosis (early apoptotic population (AnV+, PI negative) and mature apoptotic population (AnV+, PI dim). The dashed line denotes the Allocetra-OTS apoptosis and late apoptosis assays' release criteria, wherein thawed frozen populations having a % late apoptotic cells less than the dashed line and a % apoptotic cells greater than the dashed line are considered acceptable for use, that is, they meet the release criteria.

FIG. 12 presents a flow cytometry analysis of cell distribution in fresh and frozen formulations, wherein there is minimal change in the distribution of cell types between the fresh and frozen formulations. Markers used for cell identification: CD3+ cells—T-cells; CD14+ cells—monocytes; CD15+ cells—granulocytes; CD19+ cells—B-cells; and CD56+ cells—Natural Killer (NK) cells.

FIGS. 13A-13B present bar-graphs showing measures of activity of apoptotic cells (e.g., Allocetra), as a measure of percent (%) inhibition in TNF-α expression by intracellular staining and flow cytometry analysis. FIG. 13A present activity of Fresh Formulations at T=0 time vs. Frozen Formulation 1-hour post-thaw. FIG. 13B presents activity of Fresh Formulations after 96 hours storage at between 4° C.-8° C. degrees (T=96) vs. Frozen Formulation 1-hour post-thaw. The results are shown under conditions of high lipid polysaccharide (LPS) concentration (0.05 ng/ml), and low LPS concentration (0.025 ng/mL).

FIG. 14 presents a bar graph showing measures of activity of apoptotic cells (e.g., Allocetra) from Fresh and Frozen formulations, wherein activity is measured as a percent (%) inhibition of TNF-α secretion based on the evaluation of cytokine secretion by mouse TNF-α ELISA.

FIG. 15 presents the beneficial effects of Allocetra-OTS on CLP mice survival. The Kaplan-Meier survival curves of CLP mice treated with ertapenem+Allocetra-OTS in fresh or frozen thawed formulation show that Allocetra provided as either a fresh cell population or as a thawed population from a frozen formulation described herein, provided a significant increase in lifespan compared with mice not administered Allocetra. The numbers of mice in each group (N=12), representative results.

FIG. 16 shows that CLP mice treated with Frozen-thawed and Fresh Allocetra-OTS display a higher survival rate than Ertapenem (SOC) group (SOC data not shown). The graph presents the improvement in survival rate following 120 hrs post the CLP induction, compared to the standard of care group. The values in the graph represent the average from four separate experiments with bars showing SD.

FIGS. 17A and 17B present analysis of necrotic peripheral blood mononuclear cells (PBMCs). FIG. 17A shows analysis of necrotic peripheral blood mononuclear cells (PBMCs) following AnV and PI staining. The graph shows that following the necrosis induction, more than 80% of the total PBMCs were induced to necrosis phase and appeared in the AnV+, PI+ necrosis gate, which is the same gated region as cells in late apoptosis. FIG. 17B presents a bar graph showing measures of the activity of apoptotic cells (Allocetra OTS) and of necrotic cells (“Necrotic Allocetra OTS”), wherein the activity is measured as a percent (%) inhibition of TNF-α secretion based on mouse TNF-α ELISA. The graph shows that following the necrosis induction, Allocetra-OTS activity was significantly impaired.

### DETAILED DESCRIPTION

In the following detailed description, numerous specific details are set forth in order to provide a thorough understanding of the frozen formulation of apoptotic cells, methods of preparation and use thereof. However, it will be understood by those skilled in the art that storage, preparation, and use of these frozen formulations of apoptotic cells, may be practiced without these specific details. In other instances, well-known methods, procedures, and components have not been described in detail so as not to obscure the present description of the frozen formulations and uses thereof.

Cryopreservation and thawing of frozen cell populations has been associated negatively with varying extent of activation of apoptotic pathways in the cells being frozen (Matsushita et al., (203) Cell Transplantation, Vol. 12, pp. 109-121; Paasch et al. (2004) Biology of Reproduction 71, 1828-1837). The ability to freeze apoptotic cell populations as disclosed herein, wherein upon thawing the frozen formulation, a population comprising apoptotic cells that maintain functionality is retrieved, is therefore surprising and unexpected.

Disclosed herein are frozen formulations comprising apoptotic cells. Frozen formulations of apoptotic cell populations may provide a therapeutic source of apoptotic cells when needed. The ability to freeze apoptotic cells for long term storage and then to provide a frozen formulation for therapeutic use would be advantageous, wherein upon thawing the apoptotic cells maintain functional characteristics and activities useful in the treating of diseases and conditions.

In some embodiments, one characteristic of thawed frozen formulations of apoptotic cells, is that the thawed population comprises a mononuclear enriched apoptotic cell population comprising at least 35% AnnexinV+ (AnV) cells with no or low Propidium Iodide (PI) staining (apoptotic cells=Early Apoptotic cells−Annexin V+Propidium Iodide−(AnV+PI−; and Mature Apoptotic cells AnV+PI^{DIM}), and no more than 30% AnV+ and PI^{Hi} cells (Late Apoptotic cells). An apoptotic population is clearly distinguished from a late apoptotic cell population based on the lower PI fluorescence intensity. The late apoptotic cells are the population placed on the highest PI axis scale. In some embodiments, late apoptotic cells comprise cells not efficiently removed by phagocytic cells and therefore the cells become necrotic (secondary necrosis).

In some embodiments, the frozen formulation comprises a population of apoptotic mononuclear enriched cells, an isotonic crystalloid solution, and a cryoprotectant agent, wherein the formulation has a pH range of about 6.5-8.0. The mononuclear enriched cells used as the source for preparing the apoptotic cell population may comprise at least one cell type selected from monocytes, lymphocytes, T cells, B cells, and natural killer cells.

In some embodiments, the frozen formulation comprises a concentration of about 20-150×10\hat{ }⁶ cells/ml post freezing. In some embodiments, the frozen formulation comprises a concentration of about 24-150×10\hat{ }⁶ cells/ml post freezing. In some embodiments, the frozen formulation comprises a concentration of about 20-100×10\hat{ }⁶ cells/ml post freezing. In some embodiments, the frozen formulation comprises a concentration of about 24-100×10\hat{ }⁶ cells/ml post freezing. In some embodiments, the frozen formulation comprises a concentration of about 24-80×10\hat{ }⁶ cells/ml post freezing. In some embodiments, the frozen formulation comprises a concentration of about 24-60×10\hat{ }⁶ cells/ml post freezing. In some embodiments, the frozen formulation comprises a concentration of about 24-40×10\hat{ }⁶ cells/ml post freezing. In some embodiments, the frozen formulation comprises a concentration of about 25-150×10\hat{ }⁶ cells/ml post freezing. In some embodiments, the frozen formulation comprises a concentration of about 25-100×10\hat{ }⁶ cells/ml post freezing. In some embodiments, the frozen formulation comprises a concentration of about 25-80×10\hat{ }⁶ cells/ml post freezing. In some embodiments, the frozen formulation comprises a concentration of about 25-60×10\hat{ }⁶ cells/ml post freezing. In some embodiments, the frozen formulation comprises a concentration of about 25-40×10\hat{ }⁶ cells/ml post freezing. In some embodiments, the frozen formulation comprises a concentration of about 20-80×10\hat{ }⁶ cells/ml post freezing. In some embodiments, the frozen formulation comprises a concentration of about 20-60×10\hat{ }⁶ cells/ml post freezing. In some embodiments, the frozen formulation comprises a concentration of about 20-40×10\hat{ }⁶ cells/ml post freezing.

In some embodiments, the frozen formulation comprises cells, wherein at the time of freezing at least 35% of the population of apoptotic cells are in early or mature apoptosis, and less than 30% of said cells are in late apoptosis. In some embodiments, the frozen formulation comprises cells, wherein at the time of freezing at least 35% of the population of apoptotic cells are Annexin V+ (AnV+), and less than 30% of said cells are AnV+ and PI+^{High}. Immediately after thawing said frozen apoptotic cell formulations, the percentage of apoptotic cells show a gradual increase but remains within the release criteria of less than 30% late apoptotic cells. In certain embodiments, the thawed cells comprise biological activity useful in the treatment of diseases and conditions. In certain embodiments, a thawed apoptotic formulation comprises at least 35% early and mature apoptotic cells.

In some embodiments, the frozen formulation comprises an isotonic crystalloid solution and a cryoprotectant agent. In some embodiments the cryoprotectant agent comprises Dimethyl sulfoxide (DMSO). In some embodiments, the percent of DMSO used in a frozen formulation disclosed herein is less than typically used and may not be expected to provide the necessary protective actions of reducing ice formation in the cells.

In some embodiments, disclosed herein are methods of treating a subject in need comprising thawing a frozen formulation of apoptotic cells as described herein, and administering the thawed population of apoptotic cells.

### Apoptotic Cells

Apoptosis may be induced in a population of mononuclear-enriched cells. In some embodiments, mononuclear-enriched cells may be obtained directly, for example but not limited to, from a human subject using techniques such as leukapheresis. In some embodiments, mononuclear-enriched cells may be obtained indirectly, for example but not limited to, from a blood bank when peripheral blood mononuclear cells (PBMC). Methods of induction of apoptosis are well known in the art and include but are not limited to serum deprivation; incubation with a corticosteroid, for example by not limited to incubation with dexamethasone; activation of Fas or TNF receptors on the cell surface by their respective ligands, or by cross-linking these receptors with an agonist antibody; or irradiation, for example but not limited to ultraviolet B irradiation or gamma (γ) irradiation. (See for example, Roberts K. M., et al., (2004) Methods for Inducing Apoptosis. In: Perl A. (eds) Autoimmunity. Methods in Molecular Medicine™, vol 102. Humana Press; Nijhuis et al., Induction of apoptosis by heat and γ-radiation in a human lymphoid cell line; role of mitochondrial changes and caspase activation Int. J. Hyperthermia, December 2006; 22 (8): 687-698; Corinne Petit-Frère, et al., Apoptosis and cytokine release induced by ionizing or ultraviolet B radiation in primary and immortalized human keratinocytes, Carcinogenesis, Volume 21, Issue 6, June 2000, Pages 1087-1095; International Application Publication Numbers WO 2016/170541, WO 2019/038758, WO 2020/105034, WO 2014/087408, and WO 2018/225072, which are herein incorporated in their entirety).

In some embodiments, inducing apoptosis of a mononuclear enriched cell population, for example but not limited to primary lymphocytes, comprises treatment with a corticosteroid and is effected by treating the primary lymphocytes with dexamethasone. In another embodiment, inducing apoptosis of mononuclear enriched cells, for example but not limited to primary lymphocytes, is via irradiation and is effected by treating the primary lymphocytes with gamma-irradiation.

In some embodiments, an apoptotic mononuclear-enriched cell population comprises a freshly prepared apoptotic cell population. In some embodiments, a freshly prepared apoptotic mononuclear-enriched cell population is maintained on ice or at between about 2-8° C. comprises apoptotic cells. In some embodiments, maintenance of an apoptotic mononuclear-enriched population on ice is for about 24 hours. In some embodiments, maintenance of an apoptotic mononuclear-enriched population on ice is for about 48 hours. In some embodiments, maintenance of an apoptotic mononuclear-enriched population on ice is for about 72 hours. In some embodiments, maintenance of an apoptotic mononuclear-enriched population on ice is for about 96 hours. In some embodiments, maintenance of an apoptotic mononuclear-enriched population on ice is for between about 24-96 hours. In some embodiments, maintenance of an apoptotic mononuclear-enriched population at 2-8° C. is for about 24 hours. In some embodiments, maintenance of an apoptotic mononuclear-enriched population at 2-8° C. is for about 48 hours. In some embodiments, maintenance of an apoptotic mononuclear-enriched population at 2-8° C. is for about 72 hours. In some embodiments, maintenance of an apoptotic mononuclear-enriched population at 2-8° C. is for about 96 hours. In some embodiments, maintenance of an apoptotic mononuclear-enriched population at 2-8° C. is for between about 24-96 hours. In some embodiments, maintenance of an apoptotic mononuclear-enriched population is on ice for between about 24-96 hours.

In some embodiments, an apoptotic mononuclear-enriched population is stable at Rm. Temp. for up to one hour. In some embodiments, an apoptotic mononuclear-enriched population is stable at Rm. Temp. for at least one hour. In some embodiments, an apoptotic mononuclear-enriched population is stable at Rm. Temp. for more than one hour. In some embodiments, an apoptotic mononuclear-enriched population is stable at Rm. Temp. for between about 1-3 hours. In some embodiments, an apoptotic mononuclear-enriched population is stable at Rm. Temp. for about 1, 2, or 3 hours. In some embodiments, an apoptotic mononuclear-enriched population is stable at Rm. Temp. for at least 2 hours. In some embodiments, an apoptotic mononuclear-enriched population is stable at Rm. Temp. for at least 3 hours. In some embodiments, an apoptotic mononuclear-enriched population is stable at Rm. Temp. for more than 2 hours. In some embodiments, an apoptotic mononuclear-enriched population is stable at Rm. Temp. for more than 3 hours.

In some embodiments, freshly prepared apoptotic mononuclear-enriched cell populations are stored as a frozen formulation as described herein, wherein said frozen formulation comprises apoptotic cells. In some embodiments, freshly prepared apoptotic mononuclear-enriched cell populations are first maintained for a time period of up to 96 hours, for example for up to 24, 48, 72, or 96 hours, on ice and are then stored as a frozen formulation as described herein, wherein said frozen formulation comprises apoptotic cells. In some embodiments, freshly prepared apoptotic mononuclear-enriched cell populations are first maintained at between about 2-8° C. for up to 96 hours, for example for up to 24, 48, 72, or 96 hours, and are then stored as a frozen formulation as described herein, wherein said frozen formulation comprises apoptotic cells.

The state of apoptotic cell populations is an important quality attribute of the product. To determine apoptotic state, in some embodiments, Annexin V (AnV) and propidium iodide (PI) staining is performed as is well known in the art (Trahtemberg, U., Atallah, M., Krispin, A. et al. Calcium, leukocyte cell death and the use of annexin V: fatal encounters. Apoptosis 12, 1769-1780 (2007). During the initial stage of apoptosis, membrane-bound phosphatidylserine (PtdSer) translocates from the inner, cytoplasmic layer leaflet of the cell membrane to the outer, extracellular layer leaflet of the cell membrane, thus exposing PtdSer to its immediate environment and making it accessible to AnV binding. Following PtdSer exposure, the apoptotic process progresses and continues with activations of additional caspases.

Two sub-populations of apoptotic cells are derived from this dynamic process: early apoptotic cells characterized by AnV+PI⁻ staining, and mature apoptotic cells characterized by AnV+ and low PI staining (dimly PI fluorescent population due to the progression of the membrane permeabilization process which leads to gradual loss of membrane integrity). The apoptotic cell is eventually engulfed by phagocytes. If cells are not phagocytosed, the plasma membrane gradually loses its integrity. Loss of plasma membrane integrity can be detected by nonselective and massive uptake of fluorescent dyes such as PI, which leads to high intensity of PI staining on the cells. The PI enters the nucleus and intercalates within the DNA double strand and may serve as a biomarker for late apoptotic or necrotic cells. In some embodiments, late apoptotic cell populations are characterized by AnV+PI+^{High} staining. In some embodiments, late apoptotic cells comprise cells not efficiently removed by phagocytic cells and therefore the cells become necrotic (secondary necrosis).

As used herein, the terms “late apoptotic cells”, “necrotic cells”, and “secondary necrotic cells” may be used interchangeably having all the same qualities and measurements.

In some embodiments, apoptotic cell populations comprise early and mature apoptotic cell populations. In some embodiments, late apoptotic cell populations comprise necrotic (secondary necrosis) cell populations. In certain embodiments, a fresh (non-frozen) apoptotic cell population comprises at least 35% apoptotic cells (early and mature apoptosis) and less than 30% late apoptotic cells. In certain embodiments, a fresh (non-frozen) apoptotic cell population comprises at least 35% apoptotic cells (early and mature apoptosis) and less than 30% late apoptotic cells. In certain embodiments, a fresh (non-frozen) apoptotic cell population comprises at least 35% AnV+ cells (early and mature apoptosis) and less than 30% AnV+ and IP+^{High} cells. In certain embodiments, a thawed apoptotic cell population comprises at least 35% AnV+ cells (early and mature apoptosis).

A skilled artisan would appreciate that the terms “apoptotic cells”, “apoptotic cell populations”, “Allocetra”, “Allocetra-OTS”, and the like may be used interchangeably having all the same qualities and measures, and comprising greater than 35% apoptotic cells (early and mature apoptosis) and less than 30% late apoptotic cells.

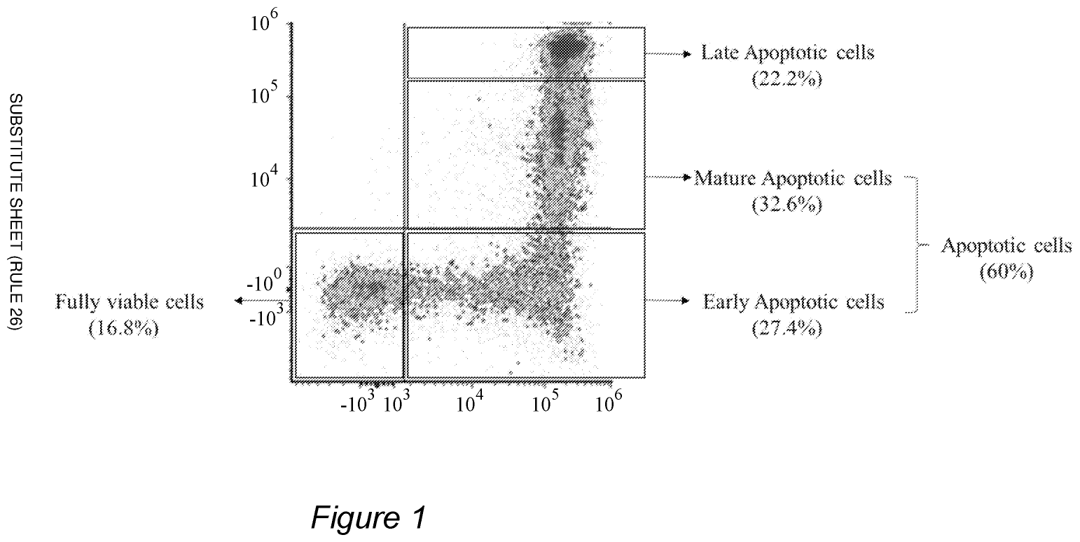

The distinction between the different apoptotic sub-population (early, mature, and late), as indicated in FIG. 1 (See, Example 1), was achieved following optimization of the analytical method and improvement in the flow cytometry sensitivity. The optimized method, with a more accurate gate strategy allows a more accurate measurement of the apoptotic population. This led to an update in release specifications. An additional characterization of late apoptotic cells is provided in Example 2.

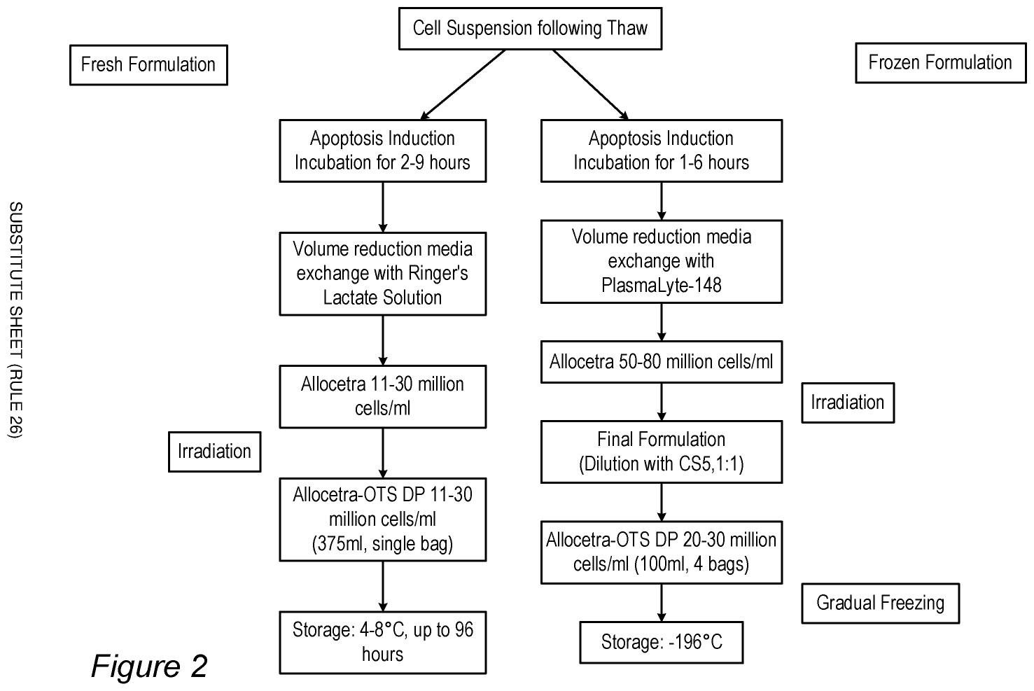

In some embodiments, freshly prepared frozen apoptotic cell populations are frozen following induction of apoptosis. In some embodiments, freshly frozen apoptotic cell populations are frozen after induction of apoptosis and a step of irradiating of said cell population (FIG. 2). As used herein, the term “freshly frozen” encompasses apoptotic cells that have been prepared and not previously frozen as an apoptotic cell population. In some embodiments, freshly prepared frozen apoptotic cells are considered cryopreserved.

As used herein, the terms “frozen apoptotic cells”, “frozen apoptotic cell populations”, “frozen Allocetra population”, “frozen apoptotic population”, “frozen formulation of apoptotic cells”, “frozen apoptotic formulation”, “cryopreserved apoptotic cells”, “cryopreserved Allocetra”, “cryopreserved apoptotic population”, “cryopreserved apoptotic cell population”, and the like may be used interchangeably having all the same qualities and meanings. Frozen formulations of apoptotic cells are prepared from apoptotic cell populations comprising at least 35% apoptotic cells (early and mature apoptosis) and less than 30% late apoptotic cells.

In some embodiments, an apoptotic mononuclear-enriched cell population comprises of a cryopreserved apoptotic cell population. When needed for a therapeutic treatment, an appropriate number of frozen bags comprising frozen formulations of apoptotic cells may be thawed.

In some embodiments, thawing comprises quickly thawing bags of cells in a pre-heated 37° C. water bath. In some embodiments, quickly thawing cells comprises thawing a bag of cells within about 1-10 minutes. In some embodiments, quickly thawing cells comprises thawing a bag of cells within about 1-5 minutes. In some embodiments, quickly thawing cells comprises thawing a bag of cells for 1, 2, 3, 4, 5, 6, 7, 8, 9, or 10 minutes. In some embodiments, quickly thawing cells comprises thawing a bag of cells within about 2-5 minutes. In some embodiments, quickly thawing cells comprises thawing a bag of cells for 2 minutes. In some embodiments, quickly thawing cells comprises thawing a bag of cells for 3 minutes. In some embodiments, quickly thawing cells comprises thawing a bag of cells for 4 minutes. In some embodiments, quickly thawing cells comprises thawing a bag of cells for 5 minutes. In some embodiments, quickly thawing cells comprises thawing a bag of cells for 6 minutes. In some embodiments, cells are thawed until no ice crystals remain in the back and are then stored until use at ambient temperature. In some embodiments, cryopreserved apoptotic cells are thawed using dry thawing techniques, and then stored at ambient temperature until use. As used herein, in some embodiments, the term “ambient temperature” may be used interchangeably with “room temperature” having all the same qualities and measures.

In some embodiments, the thawed cryopreserved apoptotic mononuclear-enriched cell population may then be administered to a subject in need immediately upon thawing. In some embodiments, the thawed cryopreserved apoptotic mononuclear-enriched cell population is maintained at ambient temperature. In some embodiments, maintenance of an apoptotic mononuclear-enriched population at ambient is for about 3 hours. In some embodiments, a thawed cryopreserved apoptotic population comprises at least 35% apoptotic cells (early and mature apoptosis). In some embodiments, a thawed cryopreserved apoptotic population comprises at least 35% apoptotic cells that are AnV+ and, PI or PI^{DIM}. In some embodiments, a thawed cryopreserved apoptotic population comprises at least 35% AnV+PI+^{DIM} apoptotic cells.

The skilled artisan would appreciate that freezing of mononuclear enriched cells for storage prior to apoptotic induction would not be considered as providing previously frozen apoptotic cells. The skilled artisan would appreciate that freezing of mononuclear enriched cells as part of the process of apoptotic induction would not be considered as providing previously frozen apoptotic cells.

In some embodiments, apoptotic mononuclear enriched cells are irradiated in a way that will decrease proliferation and/or activation of residual viable cells within the apoptotic cell population. In some embodiments, apoptotic mononuclear enriched cells are irradiated in a way that reduces the percentage of viable non-apoptotic cells in a population. In some embodiments, the percent of viable non-apoptotic cells in an inactivated apoptotic cell population is reduced to less than 50% of the population. In some embodiments, the percent of viable non-apoptotic cells in an inactivated apoptotic cell population is reduced to less than 40% of the population. In some embodiments, the percent of viable non-apoptotic cells in an inactivated apoptotic cell population is reduced to less than 30% of the population. In some embodiments, the percent of viable non-apoptotic cells in an inactivated apoptotic cell population is reduced to less than 20% of the population. In some embodiments, the percent of viable non-apoptotic cells in an inactivated apoptotic cell population is reduced to less than 10% of the population. In some embodiments, the percent of viable non-apoptotic cells in an inactivated apoptotic cell population is reduced to 0% of the population.

In another embodiment, the irradiated apoptotic mononuclear enriched cells preserve all their apoptotic-, immune modulation-, stability-properties. In another embodiment, the irradiation step uses UV radiation. In another embodiment, the radiation step uses gamma radiation. In another embodiment, the apoptotic cells comprise a decreased percent of living non-apoptotic cells, comprise a preparation having a suppressed cellular activation of any living non-apoptotic cells present within the apoptotic cell preparation, or comprise a preparation having reduced proliferation of any living non-apoptotic cells present within the apoptotic mononuclear enriched cell preparation, or any combination thereof.

In some embodiments, irradiation of apoptotic cells does not increase the population of dead (necrotic cells; PI+) compared with apoptotic cells not irradiated. In some embodiments, irradiation of apoptotic cells does not increase the population of dead cells (necrotic cells [secondary necrosis]; PI+) by more than about 1% compared with apoptotic cells not irradiated. In some embodiments, irradiation of apoptotic cells does not increase the population of dead cells (necrotic cells [secondary necrosis]; PI+) by more than about 2% compared with apoptotic cells not irradiated. In some embodiments, irradiation of apoptotic cells does not increase the population of dead cells (necrotic cells [secondary necrosis]; PI+) by more than about 3% compared with apoptotic cells not irradiated. In some embodiments, irradiation of apoptotic cells does not increase the population of dead cells (necrotic cells [secondary necrosis]; PI+) by more than about 4% compared with apoptotic cells not irradiated. In some embodiments, irradiation of apoptotic cells does not increase the population of dead cells (necrotic cells [secondary necrosis]; PI+) by more than about 5% compared with apoptotic cells not irradiated. In some embodiments, irradiation of apoptotic cells does not increase the population of dead cells (necrotic cells [secondary necrosis]; PI+) by more than about 6% compared with apoptotic cells not irradiated. In some embodiments, irradiation of apoptotic cells does not increase the population of dead cells (necrotic cells [secondary necrosis]; PI+) by more than about 7% compared with apoptotic cells not irradiated. In some embodiments, irradiation of apoptotic cells does not increase the population of dead cells (necrotic cells [secondary necrosis]; PI+) by more than about 8% compared with apoptotic cells not irradiated. In some embodiments, irradiation of apoptotic cells does not increase the population of dead cells (necrotic cells [secondary necrosis]; PI+) by more than about 9% compared with apoptotic cells not irradiated. In some embodiments, irradiation of apoptotic cells does not increase the population of dead cells (necrotic cells [secondary necrosis]; PI+) by more than about 10% compared with apoptotic cells not irradiated. In some embodiments, irradiation of apoptotic cells does not increase the population of dead cells (necrotic cells [secondary necrosis]; PI+) by more than about 15% compared with apoptotic cells not irradiated. In some embodiments, irradiation of apoptotic cells does not increase the population of dead cells (necrotic cells [secondary necrosis]; PI+) by more than about 20%, 25%, 30%, 35%, 40%, 45%, or 50% compared with apoptotic cells not irradiated.

In some embodiments, a cell population comprising a reduced or non-existent fraction of living non-apoptotic cells may in one embodiment provide a mononuclear apoptotic cell population that does not have any living/viable cells. In some embodiments, a cell population comprising a reduced or non-existent fraction of living non-apoptotic cells may in one embodiment provide a mononuclear apoptotic cell population that does not elicit GVHD in a recipient.

In some embodiments, apoptotic cells are verified by May-Giemsa-stained cytopreps. In some embodiments, viability of cells is assessed by trypan blue exclusion. In some embodiments, the apoptotic and necrotic status of the cells is confirmed by annexin V/propidium iodide staining with detection by FACS. In some embodiments, the apoptotic and or necrotic status of cells is confirmed by comparison with a known necrotic population of cells.

In some embodiments, apoptotic cells disclosed herein comprise no necrotic cells. In some embodiments, apoptotic cells disclosed herein comprise less than 30% necrotic cells. In some embodiments, apoptotic cells disclosed herein comprise less than 25% necrotic cells. In some embodiments, apoptotic cells disclosed herein comprise less than 20% necrotic cells. In some embodiments, apoptotic cells disclosed herein comprise less than 15% necrotic cells. In some embodiments, apoptotic cells disclosed herein comprise less than 10% necrotic cells. In some embodiments, apoptotic cells disclosed herein comprise less than 5% necrotic cells.

A skilled artisan would appreciate that in some embodiments the terms “apoptotic cells”, “apoptotic mononuclear-enriched cell”, “apoptotic cell population”, “apoptotic cell”, “Allocetra”, “ALC”, and “Allocetra-OTS”, and grammatical variants thereof, may be used interchangeably having all the same qualities and meanings of a population of cells comprising a population of at least 35% apoptotic cells (early and mature apoptosis) and less than 30% late apoptotic cells. In some embodiments, the population of cells comprises at least 35% of the apoptotic cells are in an early and mature state of apoptosis (at least 35% AnV+PI− (early) and AnV+PI+^{DIM} (mature)). In some embodiments, apoptotic cell populations comprise freshly prepared apoptotic cell populations. In some embodiments, apoptotic cell populations comprise frozen formulations of apoptotic cell populations. In some embodiments, apoptotic cell populations comprise apoptotic cell populations that have been thawed from frozen formulations of apoptotic cells, as described herein.

The skilled artisan would appreciate that the formulations and methods described herein, in some embodiments comprise apoptotic cells and uses thereof. In some embodiments, as described herein, apoptotic cells are HLA matched to a recipient (a subject in need of a composition comprising the apoptotic cells). In some embodiments, as described herein, apoptotic cells are not matched to a recipient (a subject in need of a composition comprising the apoptotic cells). In some embodiments, apoptotic cells are unmatched from a foreign donor. In some embodiments, the apoptotic cells not matched to a recipient of a composition comprising the apoptotic cells (a subject in need) are irradiated as described herein in detail. In some embodiments, irradiated not matched cells are termed “Allocetra-OTS”, “Allocetra”, “ALC”, or “ALC-OTS”, all having the same meaning and qualities.

In some embodiments, freshly prepared apoptotic cells are stable. A skilled artisan would appreciate that in some embodiments, stability encompasses maintaining apoptotic cell characteristics over time, for example, maintaining apoptotic cell characteristics upon storage at about 2-8° C. In some embodiments, stability comprises maintaining apoptotic cell characteristic upon storage at freezing temperatures, for example temperatures at or below 0° C. In some embodiments, stability comprises maintaining apoptotic cell characteristics upon storage at freezing temperatures, for example temperatures at about −196° C.

In some embodiments, a cryopreserved apoptotic population in its frozen state maybe stored for at least 12 months in liquid nitrogen (−196° C.). In some embodiments, a cryopreserved apoptotic population in its frozen state maybe stored for at least 1, 2, 3, 4, 5, 6, 7, 8, 9, 10, 11, or 12 months in liquid nitrogen (−196° C.). In some embodiments, a cryopreserved apoptotic population in its frozen state maybe stored for at least 1 year in liquid nitrogen (−196° C.). In certain embodiments, upon thawing the cryopreserved apoptotic population, at least 35% of the thawed population comprises apoptotic cells (early and mature state apoptotic cells).

Following thaw, the apoptotic cell population stability is maintained within release criteria specifications (at least 35% early apoptotic (AnV+PI−) and mature apoptotic (AnV+PI^{DIM}. and not more than 30% late apoptotic cells). In some embodiments, the thawed apoptotic cell population is maintained for at least 3 hours. In some embodiments, following thaw, the apoptotic cell population stability, as characterized by at least 35% apoptotic cells (early and mature state apoptotic cells), is maintained for at least 1 hour. In some embodiments, following thaw, the apoptotic cell population stability, as characterized by at least 35% apoptotic cells (early and mature state apoptotic cells), is maintained for at least 2 hours. In some embodiments, following thaw, the apoptotic cell population stability, as characterized by at least 35% apoptotic cells (early and mature state apoptotic cells), is maintained for at least 3 hours. Following thaw, the apoptotic cell population stability characterized by at least 35% apoptotic cells early and mature state apoptotic cells), is maintained for at least 3 hours.

In some embodiments, thawed apoptotic cell populations are maintained on ice. In some embodiments, thawed apoptotic cell populations are maintained in an ice-water slurry. In some embodiments, thawed apoptotic cell populations are maintained at about 4° C. In some embodiments, thawed apoptotic cell populations are maintained between about 0° C.-4° C. In some embodiments, thawed apoptotic cell populations are maintained between about 0° C.-8° C. In some embodiments, thawed apoptotic cell populations are maintained between about 4° C.-8° C.

In some embodiments, upon thawing the cryopreserved apoptotic cells, the percent of late apoptosis in the population is increased by about 15 percent, while the early apoptosis levels decrease slightly, all within release specifications. In some embodiments, upon thawing the cryopreserved apoptotic cells, the percent of late apoptosis in the population is increased by between 1-20% percent, while the early apoptosis levels decrease slightly, all within release specifications. In some embodiments, upon thawing the cryopreserved apoptotic cells, the percent of late apoptosis in the population is increased by between 1-15% percent. In some embodiments, upon thawing the cryopreserved apoptotic cells, the percent of late apoptosis in the population is increased by about 1, 2, 3, 4, 5, 6, 7, 8, 9, 10, 11, 12, 13, 14, or 15% percent. In some embodiments, upon thawing the cryopreserved apoptotic cells, the percent of late apoptosis in the population is increased by less than 15 percent. In some embodiments, upon thawing the cryopreserved apoptotic cells, the percent of late apoptosis in the population is less than 30% of the total cell population.

In some embodiments, following thaw, the apoptotic cell population stability is maintained within release criteria specification for at least 1, 2, or 3 hours. In some embodiments, following thaw, the apoptotic cell population stability is maintained within release criteria specification for more than 1, 2, or 3, hours. In some embodiments, following thaw, the apoptotic cell population stability is maintained within release criteria specification for 1, 2, or 3 hours.

In certain embodiments, apoptotic cells comprise a population of mononuclear apoptotic cells, wherein said mononuclear apoptotic cell population comprises: a decreased percent of non-quiescent non-apoptotic viable cells; a suppressed cellular activation of any living non-apoptotic cells; or a reduced proliferation of any living non-apoptotic cells; or any combination thereof.

This disclosure provides in some embodiments, a pooled mononuclear-enriched apoptotic cell population comprising mononuclear cells in an apoptotic state, wherein said pooled mononuclear-enriched apoptotic cells population comprises pooled individual mononuclear cell populations. In some embodiments, a frozen formulation of apoptotic cells as described herein, comprises a pooled mononuclear-enriched apoptotic cells population and wherein said pooled mononuclear apoptotic cell preparation comprises a decreased percent of living non-apoptotic cells, a suppressed cellular activation of any living non-apoptotic cells, or a reduced proliferation of any living non-apoptotic cells, or any combination thereof. In some embodiments, the pooled mononuclear apoptotic cells have been irradiated after induction of apoptosis and prior to freezing. In another embodiment, this disclosure provides a pooled mononuclear apoptotic cell preparation that in some embodiments, uses the white blood cell fraction (WBC) obtained from donated blood. Often this WBC fraction is discarded at blood banks or is targeted for use in research.

In some embodiments, pooled blood comprises 3^{rd} party blood from HLA matched or HLA unmatched sources, with respect to a recipient.

In some embodiments, an apoptotic cell population to be frozen, is inactivated. In another embodiment, inactivation comprises irradiation. In another embodiment, inactivation comprises T-cell receptor inactivation. In another embodiment, inactivation comprises T-cell receptor editing. In another embodiment, inactivation comprises suppressing or eliminating an immune response in said preparation. In another embodiment, inactivation comprises suppressing or eliminating cross-reactivity between multiple individual populations comprised in the preparation. In other embodiment, inactivation comprises reducing or eliminating T-cell receptor activity between multiple individual populations comprised in the preparation. In another embodiment, an inactivated cell preparation comprises a decreased percent of living non-apoptotic cells, suppressed cellular activation of any living non-apoptotic cells, or a reduce proliferation of any living non-apoptotic cells, or any combination thereof.

A skilled artisan would appreciate that the terms “apoptotic preparation” and “apoptotic population”, and grammatical variants thereof, are used interchangeably here and in some embodiments encompass all the same meanings and qualities.

In another embodiment, an inactivated cell population comprises a reduced number of non-quiescent non-apoptotic cells compared with a non-radiated cell preparation. In some embodiments, an inactivated cell population comprises 50 percent (%) of living non-apoptotic cells. In some embodiments, an inactivated cell population comprises 40% of living non-apoptotic cells. In some embodiments, an inactivated cell population comprises 30% of living non-apoptotic cells. In some embodiments, an inactivated cell population comprises 20% of living non-apoptotic cells. In some embodiments, an inactivated cell population comprises 100% of living non-apoptotic cells. In some embodiments, an inactivated cell population comprises 0% of living non-apoptotic cells.

In some embodiments, an apoptotic mononuclear-cell-enriched population comprises apoptotic cells irradiated after induction of apoptosis. In another embodiment, the irradiation comprises gamma irradiation or UV irradiation. In yet another embodiment, the irradiated preparation has a reduced number of non-quiescent non-apoptotic cells compared with a non-irradiated cell preparation.

In certain embodiments, apoptotic cells for use in frozen formulations and methods thereof, as disclosed herein, are produced in any way known in the art. In another embodiment, apoptotic cells comprised in frozen formulations disclosed herein, are autologous with a subject undergoing therapy. In another embodiment, apoptotic cells comprised in frozen formulations disclosed herein are allogeneic with a subject undergoing therapy. In another embodiment, a frozen formulation disclosed herein comprises early cells comprises apoptotic cells as is known in art.

A skilled artisan would appreciate that the term “autologous” may encompass a tissue, cell, nucleic acid molecule or polypeptide in which the donor and recipient is the same person.

A skilled artisan would appreciate that the term “allogeneic” may encompass a tissue, cell, nucleic acid molecule or polypeptide that is derived from separate individuals of the same species. In some embodiments, allogeneic donor cells are genetically distinct from the recipient.

In some embodiments, obtaining a mononuclear-enriched cell composition is affected by leukapheresis. A skilled artisan would appreciate that the term “leukapheresis” may encompass an apheresis procedure in which leukocytes are separated from the blood of a donor. In some embodiments, the blood of a donor undergoes leukapheresis and thus a mononuclear-enriched cell composition is obtained according to the production method disclosed herein. It is to be noted, that the use of at least one anticoagulant during leukapheresis is required, as is known in the art, in order to prevent clotting of the collected cells.

In some embodiments, the leukapheresis procedure is configured to allow collection of mononuclear-enriched cell composition. In some embodiments, cell collections obtained by leukapheresis comprise at least 65% mononuclear cells. In other embodiments, cell collections obtained by leukapheresis comprise at least at least 70%, or at least 80% mononuclear cells. In some embodiments, blood plasma from the cell-donor is collected in parallel to obtaining of the mononuclear-enriched cell composition. In some embodiments, about 300-600 ml of blood plasma from the cell-donor are collected in parallel to obtaining the mononuclear-enriched cell composition according to the production method disclosed herein.

In some embodiments, the apoptotic cells comprised in the frozen formulations disclosed herein comprise at least 85% mononuclear cells. In further embodiments, the apoptotic cells in the frozen formulations disclosed herein contain at least 85% mononuclear cells, 90% mononuclear cells or alternatively over 90% mononuclear cells. In some embodiments, the apoptotic cells in the frozen formulations disclosed herein comprise at least 90% mononuclear cells. In some embodiments, the apoptotic cells in the frozen formulations disclosed herein comprise at least 95% mononuclear cells.

It is to be noted that, in some embodiments, while the mononuclear-enriched cell preparation at cell collection comprises at least 65%, preferably at least 70%, most preferably at least 80% mononuclear cells, the final pharmaceutical population, following the production method of the apoptotic cells for use in the methods disclosed herein, comprises at least 85%, preferably at least 90%, most preferably at least 95% mononuclear cells.

A skilled artisan would appreciate that the term “mononuclear cells” may encompass leukocytes having a one lobed nucleus.

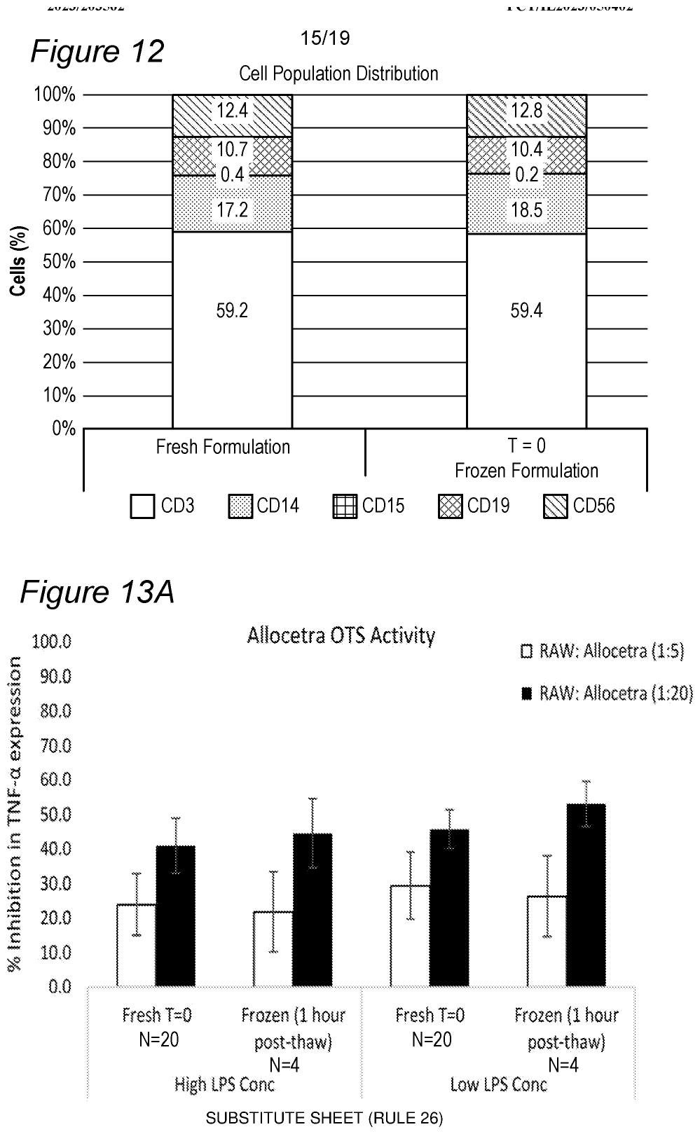

In some embodiments, the apoptotic cell preparation comprises no more than 15% CD15^{high} expressing cells. In some embodiments, the apoptotic frozen cell formulation comprises no more than 15% CD15^{high} expressing cells (See Example 1, FIG. 12).

In some embodiments, the mononuclear-enriched cell population comprises at least one cell type selected from the group consisting of lymphocytes, monocytes, T cells, B cells, and natural killer cells. In some embodiments, the mononuclear-enriched cell population comprises at least two cell types selected from the group consisting of lymphocytes, monocytes, T cells, B cells, and natural killer cells. In some embodiments, the mononuclear-enriched cell population comprises at least three cell types selected from the group consisting of lymphocytes, monocytes, T cells, B cells, and natural killer cells.

In some embodiments, the mononuclear enriched cell population comprises peripheral blood mononuclear cells (PBMC). In some embodiments, the mononuclear enriched cell population comprises a fraction of cells isolated from PBMC. In some embodiments, the mononuclear enriched cell population comprises a fraction of a single type of cells isolated from PBMC. In some embodiments, the mononuclear enriched cell population comprises a fraction of two types of cells isolated from PBMC. In some embodiments, the mononuclear enriched cell population comprises a fraction of at least a single type of cells isolated from PBMC. In some embodiments, the mononuclear enriched cell population comprises a fraction of at least two types of cells isolated from PBMC.

In some embodiments, the mononuclear-enriched cell population comprises a single cell type selected from the group consisting of lymphocytes, monocytes, T cells, B cells, and natural killer cells. In some embodiments, the mononuclear-enriched cell population comprises two cell types selected from the group consisting of lymphocytes, monocytes, T cells, B cells, and natural killer cells. In some embodiments, the mononuclear-enriched cell population comprises three cell types selected from the group consisting of lymphocytes, monocytes, T cells, B cells, and natural killer cells.

In some embodiments, the mononuclear-enriched cell population comprises lymphocytes. In some embodiments, the mononuclear-enriched cell population comprises monocytes. In some embodiments, the mononuclear-enriched cell population comprises T cells. In some embodiments, the mononuclear-enriched cell population comprises B cells. In some embodiments, the mononuclear-enriched cell population comprises natural killer cells.

In some embodiments, the mononuclear-enriched cell population comprises lymphocytes and monocytes.

In another embodiment, the mononuclear enriched cell population comprises no more than 15%, alternatively no more than 10%, typically no more than 5% polymorphonuclear leukocytes, also known as granulocytes (i.e., neutrophils, basophils and eosinophils). In another embodiment, a pooled mononuclear cell population is devoid or essentially devoid of granulocytes. In another embodiment, a pooled apoptotic cell preparation as disclosed herein comprises less than 5% polymorphonuclear leukocytes.

Functional analysis of an apoptotic cell population may in some embodiments comprise assaying the potential of an apoptotic cell population for the ability to reduce production of cytokines associated with the cytokine storm including but not limited to IL-6. Tumor Necrosis Factor alpha (TNF-α), IL-1β, and interferon-gamma (IFN-γ), alone or in combination.

In certain embodiments, the activity of an apoptotic cell population disclosed herein includes but is not limited to inhibiting pro-inflammatory cytokine expression. In certain embodiments, the activity of an apoptotic cell population disclosed herein includes but is not limited to reducing a cytokine storm. In certain embodiments, the activity of an apoptotic cell population disclosed herein includes but is not limited to reducing a cytokine release syndrome. In some embodiments, the activity of an apoptotic cell population disclosed herein includes but is not limited to reducing a cytokine storm, reducing a cytokine release syndrome or inhibiting expression of at least one pro-inflammatory cytokine, or any combination thereof.

In one embodiment, the apoptotic cells affect cytokine expression levels in macrophages. In another embodiment, the apoptotic cells reduce cytokine expression levels in macrophages. In one embodiment, the apoptotic cells suppress cytokine expression levels in macrophages. In one embodiment, the apoptotic cells inhibit cytokine expression levels in macrophages. In one embodiment, the apoptotic cells affect cytokine expression levels in dendritic cells. In another embodiment, the apoptotic cells reduce cytokine expression levels in dendritic cells. In one embodiment, the apoptotic cells suppress cytokine expression levels in dendritic cells. In one embodiment, the apoptotic cells inhibit cytokine expression levels in dendritic cells.

In some embodiment, an apoptotic cell population disclosed herein inhibits IL-6 expression in macrophages. In some embodiments, a freshly prepared apoptotic cell population exhibits inhibition of IL-6 expression in macrophages. In some embodiments, a freshly prepared apoptotic cell population maintained at between 4-8° C. for up to 96 hours exhibits inhibition of IL-6 expression in macrophages. In some embodiments, a thawed apoptotic cell population exhibits inhibition of IL-6 expression in macrophages. In some embodiments, a thawed apoptotic cell population, frozen at about −196° C. and thawed as described herein, exhibits inhibition of IL-6 expression in macrophages.

In some embodiment, an apoptotic cell population disclosed herein inhibits IL-6 expression in DCs. In some embodiments, a freshly prepared apoptotic cell population exhibits inhibition of IL-6 expression in DCs. In some embodiments, a freshly prepared apoptotic cell population maintained at between 4-8° C. for up to 96 hours exhibits inhibition of IL-6 expression in DCs. In some embodiments, a thawed apoptotic cell population exhibits inhibition of IL-6 expression in DCs. In some embodiments, a thawed apoptotic cell population, frozen at about −196° C. and thawed as described herein, exhibits inhibition of IL-6 expression in DCs.

In some embodiment, an apoptotic cell population disclosed herein inhibits TNF-α expression in macrophages. In some embodiments, a freshly prepared apoptotic cell population exhibits inhibition of TNF-α expression in macrophages. In some embodiments, a freshly prepared apoptotic cell population maintained at between 4-8° C. for up to 96 hours exhibits inhibition of TNF-α expression in macrophages. In some embodiments, a thawed apoptotic cell population exhibits inhibition of TNF-α expression in macrophages. In some embodiments, a thawed apoptotic cell population, frozen at about −196° C. and thawed as described herein, exhibits inhibition of TNF-α expression in macrophages.

In some embodiment, an apoptotic cell population disclosed herein inhibits TNF-α expression in DCs. In some embodiments, a freshly prepared apoptotic cell population exhibits inhibition of TNF-α expression in DCs. In some embodiments, a freshly prepared apoptotic cell population maintained at between 4-8° C. for up to 96 hours exhibits inhibition of TNF-α expression in DCs. In some embodiments, a thawed apoptotic cell population exhibits inhibition of TNF-α expression in DCs. In some embodiments, a thawed apoptotic cell population, frozen at about −196° C. and thawed as described herein, exhibits inhibition of TNF-α expression in DCs.

In some embodiment, an apoptotic cell population disclosed herein inhibits INF-γ expression in macrophages. In some embodiments, a freshly prepared apoptotic cell population exhibits inhibition of INF-γ expression in macrophages. In some embodiments, a freshly prepared apoptotic cell population maintained at between 4-8° C. for up to 96 hours exhibits inhibition of INF-γ expression in macrophages. In some embodiments, a thawed apoptotic cell population exhibits inhibition of INF-γ expression in macrophages. In some embodiments, a thawed apoptotic cell population, frozen at about −196° C. and thawed as described herein, exhibits inhibition of INF-γ expression in macrophages.

In some embodiment, an apoptotic cell population disclosed herein inhibits INF-γ expression in DCs. In some embodiments, a freshly prepared apoptotic cell population exhibits inhibition of INF-γ expression in DCs. In some embodiments, a freshly prepared apoptotic cell population maintained at between 4-8° C. for up to 96 hours exhibits inhibition of INF-γ expression in DCs. In some embodiments, a thawed apoptotic cell population exhibits inhibition of INF-γ expression in DCs. In some embodiments, a thawed apoptotic cell population, frozen at about −196° C. and thawed as described herein, exhibits inhibition of INF-γ expression in DCs.

In some embodiment, an apoptotic cell population disclosed herein inhibits IL-1β expression in macrophages. In some embodiments, a freshly prepared apoptotic cell population exhibits inhibition of IL-1β expression in macrophages. In some embodiments, a freshly prepared apoptotic cell population maintained at between 4-8° C. for up to 96 hours exhibits inhibition of IL-1β expression in macrophages. In some embodiments, a thawed apoptotic cell population exhibits inhibition of IL-1β expression in macrophages. In some embodiments, a thawed apoptotic cell population, frozen at about −196° C. and thawed as described herein, exhibits inhibition of IL-1β expression in macrophages.

In some embodiment, an apoptotic cell population disclosed herein inhibits IL-1β expression in DCs. In some embodiments, a freshly prepared apoptotic cell population exhibits inhibition of IL-1β expression in DCs. In some embodiments, a freshly prepared apoptotic cell population maintained at between 4-8° C. for up to 96 hours exhibits inhibition of IL-1β expression in DCs. In some embodiments, a thawed apoptotic cell population exhibits inhibition of IL-1β expression in DCs. In some embodiments, a thawed apoptotic cell population, frozen at about −196° C. and thawed as described herein, exhibits inhibition of IL-1β expression in DCs.

In some embodiments, an apoptotic cell population disclosed herein comprises an activity that reduces the expression of any of the cytokines/chemokines listed in Tables 1 and 2 below.

TABLE 1
Panel of Cytokines Increased in CRS or Cytokine Storm in Humans and/or Mice.

| | Human | Mouse model (pre-clinical) | | | | |
|---|---|---|---|---|---|---|
| | model | CAR-T | | | | |
| Cytokine | (clinical | (H) | Mouse | Not | Cells secreting | Notes/ |
| (Analyte) | trials) | origin | origin | specified | this cytokine | other |
| Flt-3L | * | | | | DC (?) | |
| Fractalkine | * | | | | APC, Endothelial | = CX3CL1, |
| | | | | | cells (?) | Neurotactin |
| | | | | | | (Mouse) |
| M-CSF | | | | | | = CSF1 |
| GM-CSF | * | | | * (in | T cell, MØ | |
| | | | | vitro) | | |
| IFN-alpha | * | | | | T cell, MØ, Monocyte | |
| IFN-beta | ? | | | ? | T cell, MØ, Monocyte | |
| IFN-gamma | * | * | | * (in | cytotoxic T cells, | |
| | | | | vitro) | helper T cells, NK | |
| | | | | | cells, MØ, | |
| | | | | | Monocyte, DC | |
| IL- 1 alpha | * | | | | Monocyte, MØ, Epithel | |
| IL- 1 beta | * | | | * | Macrophages, DCs, | |
| | | | | | fibroblasts, | |
| | | | | | endothelial cells, | |
| | | | | | hepatocytes | |
| IL- 1 R alpha | * | | | | | |
| IL- 2 | * | * | | * (in | T cells | |
| | | | | vitro) | | |
| IL- 2R alpha | * | | | | lymphocytes | |
| IL- 4 | * | * | | * (in | Th2 cells | |
| | | | | vitro) | | |
| IL- 5 | * | * | | * | T cells | |
| IL- 6 | * | | * | * | monocytes/macrophages, | |
| | | | | | dendritic cells, T | |
| | | | | | cells, fibroblasts, | |
| | | | | | keratinocytes, | |
| | | | | | endothelial cells, | |
| | | | | | adipocytes, myocytes, | |
| | | | | | mesangial cells, | |
| | | | | | and osteoblasts | |
| IL- 7 | * | | | * | In vitro by BM | |
| | | | | | stromal cells | |
| IL- 8 | * | | | | Macrophages, | |
| | | | | | monocytes | |
| IL - 9 | * | * | | | T cells, T helper | |
| IL- 10 | X | | | * (in | monocytes/macrop | |
| | | | | vitro) | hages, mast cells, B | |
| | | | | | cells, regulatory T | |
| | | | | | cells, and helper T | |
| | | | | | cells | |
| IL- 12 | * | | | * | MØ, Monocyte, DC, | = p70 |
| | | | | | activated lymphocytes, | (p40 + p35) |
| | | | | | neutrophils | |
| IL- 13 | * | * | | | T cells | |

In some embodiments, cytokines Flt-3L, Fractalkine, GM-CSF, IFN-γ, IL-1β, IL-2, IL-2Rα, IL-4, IL-5, IL-6, IL-7, IL-8, IL-9, IL-10, IL-12, and IL-13 of Table 1 are considered to be significant in CRS or cytokine storm. In another embodiment, IFN-α, IFN-β, IL-1, and IL-1Rα of Table 1 appear to be important in CRS or cytokine storm. In another embodiment, M-CSF has unknown importance. In another embodiment, any cytokine listed in Table 1, or combination thereof, may be used as a marker of apoptotic cell functionality.

TABLE 2
Panel of Cytokines Increased in CRS or Cytokine Storm in Humans and/or Mice.

| | Human | Mouse model (pre-clinical) | | | | |
|---|---|---|---|---|---|---|
| | model | CAR-T | | | | |
| Cytokine | (clinical | (H) | Mouse | Not | | Notes/ |
| (Analyte) | trials) | origin | origin | specified | Cells secreting this cytokine | other |
| IL- 15 | * | | | * | Fibroblasts, monocytes (?) | 22 |
| IL- 17 | * | | | * | T cells | |
| IL- 18 | | | | | Macrophages | |
| IL- 21 | * | | | | T helper cells, NK cells | |
| IL- 22 | * | | | | activated DC and T cells | |
| IL- 23 | | | | | | |
| IL- 25 | | | | | | Protective? |
| IL- 27 | * | | | | APC | |
| IP-10 | * | | | | Monocytes (?) | |
| MCP-1 | * | | | | Endothel, fibroblast, epithel, | = CXCL10 |
| | | | | | monocytes | |
| MCP-3 | ** | | | | PBMCs, MØ (?) | = CCL2 |
| MIP-1α | * | | | * (in vitro) | T cells | = CXCL9 |
| MIP-1β | * | | | | T cells | = CCL3 |
| PAF | ? | | | | platelets, endothelial cells, | = CCL4 |
| | | | | | neutrophils, monocytes, and | |
| | | | | | macrophages, mesangial cells | |
| PGE2 | * | | | * | Gastrointestinal mucosa and | |
| | | | | | other | |
| RANTES | * | | | | Monocytes | |
| TGF-β | * | | | * | MØ, lymphocytes, endothel, | = CCL5 |
| | | | | | platelets . . . | |
| TNF-α | * | * | * | * (in vitro) | Macrophages, NK cells, T cells | |
| TNF-αR1 | * | | | | | |
| HGF | | | | | | |
| MIG | * | | | | T cell chemoattractant, induced | |
| | | | | | by IFN-γ | |

In one embodiment, IL-15, IL-17, IL-18, IL-21, IL-22, IP-10, MCP-1, MIP-1α, MIP-1β, and TNF-α of Table 2 are considered to be significant in CRS or cytokine storm. In another embodiment, IL-27, MCP-3, PGE2, RANTES, TGF-β, TNF-αR1, and MIG of Table 2 appear to be important in CRS or cytokine storm. In another embodiment, IL-23 and IL-25 have unknown importance. In another embodiment, any cytokine listed in Table 2, or combination thereof, may be used as a marker of CRS or cytokine storm, and by extension apoptotic cell activity. In another embodiment, mouse cytokines IL-10, IL-1β, IL-2, IP-10, IL-4, IL-5, IL-6, IFNα, IL-9, IL-13, IFN-γ, IL-12p70, GM-CSF, TNF-α, MIP-1α, MIP-1β, IL-17A, IL-15/IL-15R and IL-7 appear to be important in CRS or cytokine storm.

A skilled artisan would appreciate that the term “cytokine” may encompass cytokines (e.g., interferon gamma (IFN-γ), granulocyte macrophage colony stimulating factor, tumor necrosis factor alpha), chemokines (e.g., MIP 1 alpha, MIP 1 beta, RANTES), and other soluble mediators of inflammation, such as reactive oxygen species and nitric oxide.

Efficacy of apoptotic cell populations as disclosed herein may in some embodiments be analyzed in a murine cecal ligation and puncture model (CLP). Efficacy of apoptotic cell populations as disclosed herein prior to freezing may in some embodiments be analyzed in a murine cecal ligation and puncture model (CLP). Efficacy of apoptotic cell populations as disclosed herein following thawing of a frozen formation, may in some embodiments be analyzed in a murine cecal ligation and puncture model (CLP), for example see Mevorach, D., Zuckerman, T., Reiner, I., Shimoni, A., Samuel, S., Nagler, A., Rowe, J. M., and Or, R. (2014). Single infusion of donor mononuclear apoptotic cells as prophylaxis for graft-versus-host disease in myeloablative HLA-matched allogeneic bone marrow transplantation: a phase I/IIa clinical trial. Biol. Blood Marrow Transplant. 20, 58-65; and Trahtemberg and Mevorach, 2017 ibid.

In another embodiment, the effect of apoptotic cells disclosed herein on cytokine expression levels in macrophages, dendritic cells (DCs), or a combination thereof, results in reduction of CRS. In another embodiment, the effect of apoptotic cells on cytokine expression levels in macrophages, DCs, or a combination thereof, results in reduction of severe CRS. In another embodiment, the effect of apoptotic cells on cytokine expression levels in macrophages, DCs, or a combination thereof, results in suppression of CRS. In another embodiment, the effect of apoptotic cells on cytokine expression levels in macrophages, DCs, or a combination thereof, results in suppression of severe CRS. In another embodiment, the effect of apoptotic cells on cytokine expression levels in macrophages, DCs, or a combination thereof, results in inhibition of CRS. In another embodiment, the effect of apoptotic cells on cytokine expression levels in macrophages, DCs, or a combination thereof, results in inhibition of severe CRS. In another embodiment, the effect of apoptotic cells on cytokine expression levels in macrophages, DCs, or a combination thereof, results in prevention of CRS. In another embodiment, the effect of apoptotic cells on cytokine expression levels in macrophages, DCs, or a combination thereof, results in prevention of severe CRS.

In some embodiments, administration of apoptotic cells inhibits one or more pro-inflammatory cytokines. In some embodiments, the pro-inflammatory cytokine comprises IL-1beta, IL-6, TNF-alpha, or IFN-gamma, or any combination thereof. In some embodiments, inhibition of one or more pro-inflammatory cytokines comprises downregulation of pro-inflammatory cytokines, wherein a reduced amount of one or more pro-inflammatory cytokines is secreted.

In another embodiment, apoptotic cells disclosed herein, i.e., fresh and or thawed, comprise an activity that inhibits dendritic cell maturation following exposure to TLR ligands. In another embodiment, apoptotic cells i.e., fresh and or thawed, comprise an activity that potentially creates tolerogenic dendritic cells, which in some embodiments, are capable of migration, and in some embodiments, the migration is due to CCR7.

### Frozen Formulations

Apoptotic cell populations comprising a population of apoptotic mononuclear enriched cells have been described in detail above. Herein are provided embodiments of frozen formulations comprising apoptotic cell populations.

In some embodiments, a frozen apoptotic cell formulation comprises a population of apoptotic mononuclear enriched cells; an isotonic crystalloid solution; and a cryoprotectant agent; wherein said formulation comprises a pH range of about 6.5-8.0. In some embodiments, the population of apoptotic mononuclear enriched cells frozen was freshly prepared prior to freezing. In some embodiments, the population of apoptotic mononuclear enriched cells frozen was freshly prepared and stored on ice prior to freezing. In some embodiments, the population of apoptotic mononuclear enriched cells frozen was freshly prepared and stored at between about 2-8° C. prior to freezing.

Analysis of apoptotic state is important prior to freezing the population of apoptotic mononuclear enriched cells. In some embodiments, prior to freezing the apoptotic mononuclear enriched cells comprise early and mature apoptotic cell populations. In some embodiments, prior to freezing the apoptotic mononuclear enriched cells comprise at least 35% apoptotic cells and less than 30% late apoptotic cells. In some embodiments, prior to freezing the apoptotic mononuclear enriched cells comprise at least 35% AnV+ cells and less than 30% AnV+ and PI+^{High} cells. In some embodiments, freshly prepared apoptotic cells comprise a population of mononuclear enriched apoptotic cells prepared from methods known in the art.

Density of cells at freezing may be critical to ensure the cell viability. Too high density might reduce the cell viability. Further, different cell types may require different densities at freezing, which may be tested prior to freezing. In some embodiments, the concentration of the population of apoptotic mononuclear enriched cells at freezing is about 10×10\hat{ }⁶-250×10\hat{ }⁶ cells/ml. In some embodiments, the concentration of the population of apoptotic mononuclear enriched cells at freezing is about 20×10\hat{ }⁶-100×10\hat{ }⁶ cells/ml. In some embodiments, the concentration of the population of apoptotic mononuclear enriched cells at freezing is about 20×10\hat{ }⁶-60×10\hat{ }⁶ cells/ml. In some embodiments, the concentration of the population of apoptotic mononuclear enriched cells at freezing is about 20×10\hat{ }⁶-250×10\hat{ }⁶ cells/ml. In some embodiments, the concentration of the population of apoptotic mononuclear enriched cells at freezing is about 25×10\hat{ }⁶-300×10\hat{ }⁶ cells/ml. In some embodiments, the density of the population of apoptotic mononuclear enriched cells at freezing is about 24×10⁶-50×10⁶ cells/ml. In some embodiments, the density of the population of apoptotic mononuclear enriched cells at freezing is about 24×10⁶-40×10⁶ cells/ml. In some embodiments, the density of the population of apoptotic mononuclear enriched cells at freezing is about 44×10⁶-50×10⁶ cells/ml. In some embodiments, the density of the population of apoptotic mononuclear enriched cells at freezing is about 24×10⁶-35×10⁶ cells/ml. In some embodiments, the density of the population of apoptotic mononuclear enriched cells at freezing is about 20×10⁶-40×10⁶ cells/ml. In some embodiments, the density of the population of apoptotic mononuclear enriched cells at freezing is about 20×10⁶-100×10⁶ cells/ml. In some embodiments, the density of the population of apoptotic mononuclear enriched cells at freezing is about 20×10⁶-35×10⁶ cells/ml. In some embodiments, the density of the population of apoptotic mononuclear enriched cells at freezing is about 25×10⁶-35×10⁶ cells/ml. In some embodiments, the density of the population of apoptotic mononuclear enriched cells at freezing is about 35×10⁶-45×10⁶ cells/ml. In some embodiments, the density of the population of apoptotic mononuclear enriched cells at freezing is about 35×10⁶-40×10⁶ cells/ml. In some embodiments, the density of the population of apoptotic mononuclear enriched cells at freezing is about 40×10⁶-50×10⁶ cells/ml. In some embodiments, the density of the population of apoptotic mononuclear enriched cells at freezing is about 40×10⁶-60×10⁶ cells/ml.

As described above, the mononuclear enriched cells from which apoptotic cells to be frozen are produced may in certain embodiments comprise at least one cell type selected from monocytes, lymphocytes, T cells, B cells, and natural killer cells. In some embodiments, the mononuclear enriched cells from which apoptotic cells to be frozen are produced comprise PBMC. In some embodiments, the mononuclear enriched cells from which apoptotic cells to be frozen are produced comprise a fraction of PBMC comprising at least one cell type. In some embodiments, the mononuclear enriched cells from which apoptotic cells to be frozen are produced comprise a fraction of PBMC comprising at least two cell types. In some embodiments, the mononuclear enriched cells from which apoptotic cells to be frozen are produced comprise a single cell type selected from monocytes, lymphocytes, T cells, B cells, and natural killer cells.

In some embodiments, a frozen formulation of apoptotic mononuclear enriched cells comprises a pooled mononuclear apoptotic cell preparation. In some embodiments, a pooled mononuclear apoptotic cell preparation comprises mononuclear cells in an apoptotic state, wherein said pooled mononuclear apoptotic cells comprise a decreased percent of living non-apoptotic cells, a suppressed cellular activation of any living non-apoptotic cells, or a reduced proliferation of any living non-apoptotic cells, or any combination thereof. In certain embodiments, the pooled mononuclear enriched apoptotic cells have been irradiated. In some embodiments, disclosed herein is a pooled mononuclear enriched apoptotic cell preparation that originates from the white blood cell fraction (WBC) obtained from donated blood.

The population of apoptotic mononuclear enriched cells for freezing may be produced by any method known in the art including but not limited to inducing apoptosis in a mononuclear cell population using methylprednisolone as described in detail in International Publication No. WO 2014/087408 at Example 14, International Publication No. WO 2019/038758 at Example 1, and International Publication No. WO 2016/170541 at Examples 1, 2, 3, 5, and 6, which are all hereby incorporated in their entirety.

Crystalloid fluids, i.e., crystalloid solutions, comprise an aqueous solution of mineral salts and other small, water-soluble molecules. Crystalloid solutions may in some embodiments be isotonic to human plasma. In some embodiments, crystalloid solutions approximate concentrations of various solutes found in plasma and do not exert an osmotic effect in vivo. Crystalloid solutions may function to expand intravascular volume without disturbing ion concentration or causing significant fluid shifts between intracellular, intravascular, and interstitial spaces. In some embodiments, a frozen formulation of apoptotic cells disclosed herein comprises an isotonic crystalloid solution.

In some embodiments, the isotonic crystalloid solution present in a frozen formulation of apoptotic mononuclear cells comprises sodium chloride (NaCl), potassium chloride (KCl), magnesium chloride (MgCl₂), sodium acetate (NaCH₃COO, also abbreviated NaOAc), or sodium gluconate (NaC₆H₁₁O₇), or any combination thereof. In some embodiments, the isotonic crystalloid solution present in a frozen formulation of apoptotic mononuclear cells comprises sodium chloride (NaCl). In some embodiments, the isotonic crystalloid solution present in a frozen formulation of apoptotic mononuclear cells comprises potassium chloride (KCl). In some embodiments, the isotonic crystalloid solution present in a frozen formulation of apoptotic mononuclear cells comprises magnesium chloride (MgCl₂). In some embodiments, the isotonic crystalloid solution present in a frozen formulation of apoptotic mononuclear cells comprises sodium acetate (NaCH₃COO, also abbreviated NaOAc). In some embodiments, the isotonic crystalloid solution present in a frozen formulation of apoptotic mononuclear cells comprises or sodium gluconate (NaC₆H₁₁O₇), or any combination thereof.

In some embodiments, the isotonic crystalloid solution present in a frozen formulation of apoptotic mononuclear cells comprises 140 mmol/L sodium, 5 mmol/L potassium, 1.5 mmol/L magnesium, 98 mmol/L chloride, 27 mmol/L acetate, and 23 mmol/L gluconate. In some embodiments, the isotonic crystalloid solution present in a frozen formulation of apoptotic mononuclear cells comprises PLASMA-LYTE 148 (Baxter USA), also known as Normasol® (Molnlycke Sweden).

In some embodiments, the isotonic crystalloid solution present in a frozen formulation of apoptotic mononuclear cells comprises a normal saline solution (0.9% NaCl solution), a lactated Ringer's/Hartman's solution (lactate buffered solution), an acetate buffered solution, an acetate and lactate buffered solution, an acetate and gluconate buffered solution, 5% dextrose in water, or 10% dextrose in water, or any combination thereof. In some embodiments, the isotonic crystalloid solution present in a frozen formulation of apoptotic mononuclear cells comprises a normal saline solution (0.9% NaCl solution). In some embodiments, the isotonic crystalloid solution present in a frozen formulation of apoptotic mononuclear cells comprises a lactated Ringer's/Hartman's solution (lactate buffered solution). In some embodiments, the isotonic crystalloid solution present in a frozen formulation of apoptotic mononuclear cells comprises an acetate buffered solution. In some embodiments, the isotonic crystalloid solution present in a frozen formulation of apoptotic mononuclear cells comprises an acetate and lactate buffered solution. In some embodiments, the isotonic crystalloid solution present in a frozen formulation of apoptotic mononuclear cells comprises an acetate and gluconate buffered solution. In some embodiments, the isotonic crystalloid solution present in a frozen formulation of apoptotic mononuclear cells comprises 5% dextrose in water. In some embodiments, the isotonic crystalloid solution present in a frozen formulation of apoptotic mononuclear cells comprises 10% dextrose in water.

In some embodiments, the isotonic crystalloid solution present in a frozen formulation of apoptotic mononuclear cells is serum free. In some embodiments, the isotonic crystalloid solution present in a frozen formulation of apoptotic mononuclear cells is protein free. In some embodiments, the isotonic crystalloid solution present in a frozen formulation of apoptotic mononuclear cells is calcium (Ca⁺⁺) free. In some embodiments, the isotonic crystalloid solution present in a frozen formulation of apoptotic mononuclear cells does not contain antimicrobial agents.

Ensuring the proper acid-base balance for the frozen formulation is important. In some embodiments, the pH of the freezing formulation affects the quality of the cells at thawing. Maintenance of proper cold-dependent ion ratios, control of pH at lowered temperature, and prevention of the formation of free radicals, are critical elements during cryopreservation of cells and may affect the quality of the cells at thawing.

In some embodiments, the pH of a frozen formulation comprising apoptotic mononuclear enriched cells is at about pH 6.5-8.0. In some embodiments, the pH of a frozen formulation comprising apoptotic mononuclear enriched cells is at about pH 7.0-8.0. In some embodiments, the pH of a frozen formulation comprising apoptotic mononuclear enriched cells is at about pH 6.5-7.5. In some embodiments, the pH of a frozen formulation comprising apoptotic mononuclear enriched cells is at about pH 7.0-7.5. In some embodiments, the pH of a frozen formulation comprising apoptotic mononuclear enriched cells is at about pH 7.5-8.0. In some embodiments, the pH of a frozen formulation comprising apoptotic mononuclear enriched cells is at about pH 6.5, 6.6, 6.7, 6.8, 6.9, 7.0, 7.1, 7.2, 7.3, 7.4, 7.5, 7.6, 7.7, 7.8, 7.9, or 8.0. In some embodiments, the pH of a frozen formulation comprising apoptotic mononuclear enriched cells is pH 6.5, 6.6, 6.7, 6.8, 6.9, 7.0, 7.1, 7.2, 7.3, 7.4, 7.5, 7.6, 7.7, 7.8, 7.9, or 8.0.

In some embodiments, the pH of a frozen formulation comprising apoptotic mononuclear enriched cells is at about pH 6.5. In some embodiments, the pH of a frozen formulation comprising apoptotic mononuclear enriched cells is at about pH 6.6. In some embodiments, the pH of a frozen formulation comprising apoptotic mononuclear enriched cells is at about pH 6.7. In some embodiments, the pH of a frozen formulation comprising apoptotic mononuclear enriched cells is at about pH 6.8. In some embodiments, the pH of a frozen formulation comprising apoptotic mononuclear enriched cells is at about pH 6.9. In some embodiments, the pH of a frozen formulation comprising apoptotic mononuclear enriched cells is at about pH 7.0. In some embodiments, the pH of a frozen formulation comprising apoptotic mononuclear enriched cells is at about pH 7.1. In some embodiments, the pH of a frozen formulation comprising apoptotic mononuclear enriched cells is at about pH 7.2. In some embodiments, the pH of a frozen formulation comprising apoptotic mononuclear enriched cells is at about pH 7.3. In some embodiments, the pH of a frozen formulation comprising apoptotic mononuclear enriched cells is at about pH 7.4. In some embodiments, the pH of a frozen formulation comprising apoptotic mononuclear enriched cells is at about pH 7.5. In some embodiments, the pH of a frozen formulation comprising apoptotic mononuclear enriched cells is at about pH 7.6. In some embodiments, the pH of a frozen formulation comprising apoptotic mononuclear enriched cells is at about pH 7.7. In some embodiments, the pH of a frozen formulation comprising apoptotic mononuclear enriched cells is at about pH 7.8. In some embodiments, the pH of a frozen formulation comprising apoptotic mononuclear enriched cells is at about pH 7.9. In some embodiments, the pH of a frozen formulation comprising apoptotic mononuclear enriched cells is at about pH 8.0.

In some embodiments, the pH of a frozen formulation comprising apoptotic mononuclear enriched cells is pH 6.5. In some embodiments, the pH of a frozen formulation comprising apoptotic mononuclear enriched cells is pH 6.6. In some embodiments, the pH of a frozen formulation comprising apoptotic mononuclear enriched cells is pH 6.7. In some embodiments, the pH of a frozen formulation comprising apoptotic mononuclear enriched cells is pH 6.8. In some embodiments, the pH of a frozen formulation comprising apoptotic mononuclear enriched cells is pH 6.9. In some embodiments, the pH of a frozen formulation comprising apoptotic mononuclear enriched cells is pH 7.0. In some embodiments, the pH of a frozen formulation comprising apoptotic mononuclear enriched cells is pH 7.1. In some embodiments, the pH of a frozen formulation comprising apoptotic mononuclear enriched cells is pH 7.2. In some embodiments, the pH of a frozen formulation comprising apoptotic mononuclear enriched cells is pH 7.3. In some embodiments, the pH of a frozen formulation comprising apoptotic mononuclear enriched cells is pH 7.4. In some embodiments, the pH of a frozen formulation comprising apoptotic mononuclear enriched cells is pH 7.5. In some embodiments, the pH of a frozen formulation comprising apoptotic mononuclear enriched cells is pH 7.6. In some embodiments, the pH of a frozen formulation comprising apoptotic mononuclear enriched cells is pH 7.7. In some embodiments, the pH of a frozen formulation comprising apoptotic mononuclear enriched cells is pH 7.8. In some embodiments, the pH of a frozen formulation comprising apoptotic mononuclear enriched cells is pH 7.9. In some embodiments, the pH of a frozen formulation comprising apoptotic mononuclear enriched cells is pH 8.0.

Cryoprotectants are used during cryopreservation to prevent cells from damage due to freezing, for example due to ice formation. It is important that cryoprotectants be non- or minimally toxic, able to penetrate cell membranes easily, and able to bind either with electrolytes (to increase concentration in the freezing process) or with water molecules (to delay freezing).

In some embodiments, the cryoprotectant agent present in a frozen formulation of apoptotic mononuclear enriched cells comprises glycerol, ethylene glycol, propylene glycol, or dimethyl sulfoxide (DMSO). In certain embodiments, the cryoprotectant agent present in a frozen formulation of apoptotic mononuclear enriched cells comprises glycerol. In certain embodiments, the cryoprotectant agent present in a frozen formulation of apoptotic mononuclear cells comprises ethylene glycol. In certain embodiments, the cryoprotectant agent present in a frozen formulation of apoptotic mononuclear cells comprises propylene glycol. In certain embodiments, the cryoprotectant agent present in a frozen formulation of apoptotic mononuclear cells comprises dimethyl sulfoxide (DMSO). Commercial cryoprotectants available include but are not limited to PentaHibe® (Pharmacosmos, Denmark) Cryostor® CS5 and Cryostor® CD10 (Merck KGaA, Darmstadt, Germany), and mFreSR™ (Stemcell Technologies, USA).

While a typical cryoprotectant concentration of about 5% to 15% (vol/vol) is usually required to permit survival of a substantial fraction of isolated cells after freezing and thawing from liquid nitrogen temperature (−196°), as exemplified in Example 1, surprisingly the survival of a substantial fraction of apoptotic mononuclear enriched cells and maintenance of their functionality was obtains using concentrations of cryoprotectant less than 5%.

In some embodiments, the percent (%) cryoprotectant agent present in a frozen formulation of apoptotic mononuclear enriched cells comprises between about 2.0%-10% (vol/vol). In some embodiments, the % cryoprotectant agent present in a frozen formulation of apoptotic mononuclear enriched cells comprises between about 2.5%-10% (vol/vol). In some embodiments, the % cryoprotectant agent present in a frozen formulation of apoptotic mononuclear enriched cells comprises between about 2.5%-5% (vol/vol). In some embodiments, the % cryoprotectant agent present in a frozen formulation of apoptotic mononuclear enriched cells comprises between about 5%-10% (vol/vol). In some embodiments, the % cryoprotectant agent present in a frozen formulation of apoptotic mononuclear enriched cells comprises between about 5%-15% (vol/vol).

In some embodiments, the % cryoprotectant agent present in a frozen formulation of apoptotic mononuclear enriched cells comprises less than 2.0% (vol/vol). In some embodiments, the % cryoprotectant agent present in a frozen formulation of apoptotic mononuclear enriched cells comprises less than 2.5% (vol/vol). In some embodiments, the % cryoprotectant agent present in a frozen formulation of apoptotic mononuclear enriched cells comprises less than 3.0% (vol/vol). In some embodiments, the % cryoprotectant agent present in a frozen formulation of apoptotic mononuclear enriched cells comprises less than 3.5% (vol/vol). In some embodiments, the % cryoprotectant agent present in a frozen formulation of apoptotic mononuclear enriched cells comprises less than 4.0% (vol/vol). In some embodiments, the % cryoprotectant agent present in a frozen formulation of apoptotic mononuclear enriched cells comprises less than 4.5% (vol/vol). In some embodiments, the % cryoprotectant agent present in a frozen formulation of apoptotic mononuclear enriched cells comprises less than 5.0% (vol/vol). In some embodiments, the % cryoprotectant agent present in a frozen formulation of apoptotic mononuclear enriched cells comprises less than 5.5% (vol/vol). In some embodiments, the % cryoprotectant agent present in a frozen formulation of apoptotic mononuclear enriched cells comprises less than 6.0% (vol/vol). In some embodiments, the % cryoprotectant agent present in a frozen formulation of apoptotic mononuclear enriched cells comprises less than 6.5% (vol/vol). In some embodiments, the % cryoprotectant agent present in a frozen formulation of apoptotic mononuclear enriched cells comprises less than 7.0% (vol/vol). In some embodiments, the % cryoprotectant agent present in a frozen formulation of apoptotic mononuclear enriched cells comprises less than 7.5% (vol/vol). In some embodiments, the % cryoprotectant agent present in a frozen formulation of apoptotic mononuclear enriched cells comprises less than 8.0% (vol/vol). In some embodiments, the % cryoprotectant agent present in a frozen formulation of apoptotic mononuclear enriched cells comprises less than 8.5% (vol/vol). In some embodiments, the % cryoprotectant agent present in a frozen formulation of apoptotic mononuclear enriched cells comprises less than 9.0% (vol/vol). In some embodiments, the % cryoprotectant agent present in a frozen formulation of apoptotic mononuclear enriched cells comprises less than 9.5% (vol/vol). In some embodiments, the % cryoprotectant agent present in a frozen formulation of apoptotic mononuclear enriched cells comprises less than 10% (vol/vol).

In some embodiments, the % cryoprotectant agent present in a frozen formulation of apoptotic mononuclear enriched cells comprises equal to or less than about 2.0% (vol/vol). In some embodiments, the % cryoprotectant agent present in a frozen formulation of apoptotic mononuclear enriched cells comprises equal to or less than about 2.5% (vol/vol). In some embodiments, the % cryoprotectant agent present in a frozen formulation of apoptotic mononuclear enriched cells comprises equal to or less than about 3.0% (vol/vol). In some embodiments, the % cryoprotectant agent present in a frozen formulation of apoptotic mononuclear enriched cells comprises equal to or less than about 3.5% (vol/vol). In some embodiments, the % cryoprotectant agent present in a frozen formulation of apoptotic mononuclear enriched cells comprises equal to or less than about 4.0% (vol/vol). In some embodiments, the % cryoprotectant agent present in a frozen formulation of apoptotic mononuclear enriched cells comprises equal to or less than about 4.5% (vol/vol). In some embodiments, the % cryoprotectant agent present in a frozen formulation of apoptotic mononuclear enriched cells comprises equal to or less than about 5.0% (vol/vol). In some embodiments, the % cryoprotectant agent present in a frozen formulation of apoptotic mononuclear enriched cells comprises equal to or less than about 5.5% (vol/vol). In some embodiments, the % cryoprotectant agent present in a frozen formulation of apoptotic mononuclear enriched cells comprises equal to or less than about 6.0% (vol/vol). In some embodiments, the % cryoprotectant agent present in a frozen formulation of apoptotic mononuclear enriched cells comprises equal to or less than about 6.5% (vol/vol). In some embodiments, the % cryoprotectant agent present in a frozen formulation of apoptotic mononuclear enriched cells comprises equal to or less than about 7.0% (vol/vol). In some embodiments, the % cryoprotectant agent present in a frozen formulation of apoptotic mononuclear enriched cells comprises equal to or less than about 7.5% (vol/vol). In some embodiments, the % cryoprotectant agent present in a frozen formulation of apoptotic mononuclear enriched cells comprises equal to or less than about 8.0% (vol/vol). In some embodiments, the % cryoprotectant agent present in a frozen formulation of apoptotic mononuclear enriched cells comprises equal to or less than about 8.5% (vol/vol). In some embodiments, the % cryoprotectant agent present in a frozen formulation of apoptotic mononuclear enriched cells comprises equal to or less than about 9.0% (vol/vol). In some embodiments, the % cryoprotectant agent present in a frozen formulation of apoptotic mononuclear enriched cells comprises equal to or less than about 9.5% (vol/vol). In some embodiments, the % cryoprotectant agent present in a frozen formulation of apoptotic mononuclear enriched cells comprises equal to or less than about 10% (vol/vol).

In some embodiments, the % cryoprotectant agent present in a frozen formulation of apoptotic mononuclear enriched cells comprises about 2.0% (vol/vol). In some embodiments, the % cryoprotectant agent present in a frozen formulation of apoptotic mononuclear enriched cells comprises about 2.5% (vol/vol). In some embodiments, the % cryoprotectant agent present in a frozen formulation of apoptotic mononuclear enriched cells comprises about 3.0% (vol/vol). In some embodiments, the % cryoprotectant agent present in a frozen formulation of apoptotic mononuclear enriched cells comprises about 3.5% (vol/vol). In some embodiments, the % cryoprotectant agent present in a frozen formulation of apoptotic mononuclear enriched cells comprises about 4.0% (vol/vol). In some embodiments, the % cryoprotectant agent present in a frozen formulation of apoptotic mononuclear enriched cells comprises about 4.5% (vol/vol). In some embodiments, the % cryoprotectant agent present in a frozen formulation of apoptotic mononuclear enriched cells comprises about 5.0% (vol/vol). In some embodiments, the % cryoprotectant agent present in a frozen formulation of apoptotic mononuclear enriched cells comprises about 5.5% (vol/vol). In some embodiments, the % cryoprotectant agent present in a frozen formulation of apoptotic mononuclear enriched cells comprises about 6.0% (vol/vol). In some embodiments, the % cryoprotectant agent present in a frozen formulation of apoptotic mononuclear enriched cells comprises about 6.5% (vol/vol). In some embodiments, the % cryoprotectant agent present in a frozen formulation of apoptotic mononuclear enriched cells comprises about 7.0% (vol/vol). In some embodiments, the % cryoprotectant agent present in a frozen formulation of apoptotic mononuclear enriched cells comprises about 7.5% (vol/vol). In some embodiments, the % cryoprotectant agent present in a frozen formulation of apoptotic mononuclear enriched cells comprises about 8.0% (vol/vol). In some embodiments, the % cryoprotectant agent present in a frozen formulation of apoptotic mononuclear enriched cells comprises about 8.5% (vol/vol). In some embodiments, the % cryoprotectant agent present in a frozen formulation of apoptotic mononuclear enriched cells comprises about 9.0% (vol/vol). In some embodiments, the % cryoprotectant agent present in a frozen formulation of apoptotic mononuclear enriched cells comprises about 9.5% (vol/vol). In some embodiments, the % cryoprotectant agent present in a frozen formulation of apoptotic mononuclear enriched cells comprises about 10% (vol/vol).

In some embodiments, the cryoprotectant agent comprises DMSO and the % DMSO present in a frozen formulation of apoptotic mononuclear enriched cells comprises about 2.0%, 2.5%, 3.0%, 3.5%, 4.0%, 4.5%, 5%, 5.5%, 6.0%, 6.5%, 7.0%, 7.5%, 8.0%, 8.5%, 9.0%, 9.5%, or 10.0% (vol/vol). In some embodiments, the cryoprotectant agent comprises DMSO and the % DMSO present in a frozen formulation of apoptotic mononuclear enriched cells comprises about 2.0%. In some embodiments, % DMSO present in a frozen formulation of apoptotic mononuclear enriched cells comprises about 2.5%. In some embodiments, % DMSO present in a frozen formulation of apoptotic mononuclear enriched cells comprises about 3.0%. In some embodiments, the % DMSO present in a frozen formulation of apoptotic mononuclear enriched cells comprises about 3.5%. In some embodiments, % DMSO present in a frozen formulation of apoptotic mononuclear enriched cells comprises about 4.0%. In some embodiments, % DMSO present in a frozen formulation of apoptotic mononuclear enriched cells comprises about 4.5%. In some embodiments, % DMSO present in a frozen formulation of apoptotic mononuclear enriched cells comprises about 5%. In some embodiments, % DMSO present in a frozen formulation of apoptotic mononuclear enriched cells comprises about 5.5%. In some embodiments, % DMSO present in a frozen formulation of apoptotic mononuclear enriched cells comprises about 6.0%. In some embodiments, % DMSO present in a frozen formulation of apoptotic mononuclear enriched cells comprises about 6.5%. In some embodiments, % DMSO present in a frozen formulation of apoptotic mononuclear enriched cells comprises about 7.0%. In some embodiments, % DMSO present in a frozen formulation of apoptotic mononuclear enriched cells comprises about 7.5%. In some embodiments, % DMSO present in a frozen formulation of apoptotic mononuclear enriched cells comprises about 8.0%. In some embodiments, % DMSO present in a frozen formulation of apoptotic mononuclear enriched cells comprises about 8.5%. In some embodiments, % DMSO present in a frozen formulation of apoptotic mononuclear enriched cells comprises about 9.0%. In some embodiments, % DMSO present in a frozen formulation of apoptotic mononuclear enriched cells comprises about 9.5%. In some embodiments, % DMSO present in a frozen formulation of apoptotic mononuclear enriched cells comprises about 10.0% (vol/vol).

### Preparation of Frozen Formulations

The methods for preparation of frozen formulations described herein are directed to freezing populations of apoptotic mononuclear enriched cell populations, as described herein.

In certain embodiments, a method of preparing a frozen apoptotic cell formulation comprises steps: (a) resuspending an apoptotic mononuclear enriched population of cells in an isotonic crystalloid solution having a pH of about 6.5-8.0; (b) mixing said resuspend apoptotic cells of step (a) with a cryoprotectant agent; and (c) gradually freezing said mix of step (b) to a temperature of about −196° C.

In some embodiments, the apoptotic mononuclear enriched population of cells used in the method of preparing a frozen formulation comprises an apoptotic mononuclear enriched population independent of the method of induction of apoptosis. In some embodiments, the apoptotic mononuclear enriched population of cells used in the method of preparing a frozen formulation comprises an apoptotic mononuclear enriched population produced using methylprednisolone for induction of apoptosis as is described in detail International Publication No. WO 2014/087408 at Example 14, International Publication No. WO 2019/038758 at Example 1, and International Publication No. WO 2016/170541 at Examples 1, 2, 3, 5, and 6, which are all hereby incorporated in their entirety.

In some embodiments, the apoptotic mononuclear enriched population of cells used in the method of preparing a frozen formulation comprises at least one cell type selected from the group consisting of lymphocytes, monocytes, T cells, B cells, and natural killer cells. In some embodiments, the apoptotic mononuclear enriched population of cells used in the method of preparing a frozen formulation comprises at least two cell types selected from the group consisting of lymphocytes, monocytes, T cells, B cells, and natural killer cells. In some embodiments, the apoptotic mononuclear enriched population of cells used in the method of preparing a frozen formulation comprises at least three cell types selected from the group consisting of lymphocytes, monocytes, T cells, B cells, and natural killer cells.

In some embodiments, the apoptotic mononuclear enriched population of cells used in the method of preparing a frozen formulation comprises peripheral blood mononuclear cells (PBMC). In some embodiments, the apoptotic mononuclear enriched population of cells used in the method of preparing a frozen formulation comprises a fraction of cells isolated from PBMC. In some embodiments, the apoptotic mononuclear enriched population of cells used in the method of preparing a frozen formulation comprises a fraction of a single type of cells isolated from PBMC. In some embodiments, the apoptotic mononuclear enriched population of cells used in the method of preparing a frozen formulation comprises a fraction of two types of cells isolated from PBMC. In some embodiments, the apoptotic mononuclear enriched population of cells used in the method of preparing a frozen formulation comprises a fraction of at least a single type of cells isolated from PBMC. In some embodiments, the apoptotic mononuclear enriched population of cells used in the method of preparing a frozen formulation comprises a fraction of at least two types of cells isolated from PBMC.

In some embodiments, the apoptotic mononuclear enriched population of cells used in the method of preparing a frozen formulation comprises a single cell type selected from the group consisting of lymphocytes, monocytes, T cells, B cells, and natural killer cells. In some embodiments, the apoptotic mononuclear enriched population of cells used in the method of preparing a frozen formulation comprises two cell types selected from the group consisting of lymphocytes, monocytes, T cells, B cells, and natural killer cells. In some embodiments, the apoptotic mononuclear enriched population of cells used in the method of preparing a frozen formulation comprises three cell types selected from the group consisting of lymphocytes, monocytes, T cells, B cells, and natural killer cells.

In some embodiments, the apoptotic mononuclear enriched population of cells used in the method of preparing a frozen formulation comprises lymphocytes. In some embodiments, the apoptotic mononuclear enriched population of cells used in the method of preparing a frozen formulation comprises monocytes. In some embodiments, the apoptotic mononuclear enriched population of cells used in the method of preparing a frozen formulation comprises T cells. In some embodiments, the apoptotic mononuclear enriched population of cells used in the method of preparing a frozen formulation comprises B cells. In some embodiments, the apoptotic mononuclear enriched population of cells used in the method of preparing a frozen formulation comprises natural killer cells.

In some embodiments, the apoptotic mononuclear enriched population of cells used in the method of preparing a frozen formulation comprises lymphocytes and monocytes.

In some embodiments, the apoptotic mononuclear enriched population of cells used in the method of preparing a frozen formulation comprises no more than 15%, alternatively no more than 10%, typically no more than 5% polymorphonuclear leukocytes, also known as granulocytes (i.e., neutrophils, basophils and eosinophils). In some embodiments, the apoptotic mononuclear enriched population of cells used in the method of preparing a frozen formulation comprises a pooled mononuclear cell population. In some embodiments, the apoptotic mononuclear enriched population of cells used in the method of preparing a frozen formulation comprises a pooled mononuclear cell population that is devoid or essentially devoid of granulocytes. In some embodiments, the apoptotic mononuclear enriched population of cells used in the method of preparing a frozen formulation comprises a pooled mononuclear cell population that has less than 5% polymorphonuclear leukocytes.

In some embodiments of preparing the frozen formulation, said mononuclear enriched cells comprise at least one cell type selected from monocytes, lymphocytes, T cells, B cells, and natural killer cells.

In some embodiments of preparing the frozen formulation, said at least one cell type comprises a single cell type selected from monocytes, lymphocytes. T cells, B cells, and natural killer cells.

FIG. 2 provides a flow chart comparing the methods of preparing fresh and frozen formulations comprising apoptotic mononuclear enriched cell. In both methods, once apoptotic induction is completed cells are resuspended in an isotonic crystalloid solution. In the case of a preparation that will be freshly used, or used after storage at about 4-8° C. (for up to about 96 hours), the isotonic crystalloid solution used is Ringer's lactate solution. In certain embodiments, as shown in FIG. 2, the isotonic crystalloid solution used for an apoptotic population of cells destined to be frozen, Plasma-Lyte was used.

In some embodiments of preparing a frozen formulation of an apoptotic mononuclear enriched population of cells, the isotonic crystalloid solution used for resuspension comprises sodium chloride (NaCl), potassium chloride (KCl), magnesium chloride (MgCl₂), sodium acetate (NaCH₃COO, also abbreviated NaOAc), or sodium gluconate (NaC₆H₁₁O₇), or any combination thereof. In some embodiments, the isotonic crystalloid solution used for resuspension in a method of preparing a frozen formulation comprises sodium chloride (NaCl). In some embodiments, the isotonic crystalloid solution used for resuspension in a method of preparing a frozen formulation comprises potassium chloride (KCl). In some embodiments, the isotonic crystalloid solution used for resuspension in a method of preparing a frozen formulation comprises magnesium chloride (MgCl₂). In some embodiments, the isotonic crystalloid solution used for resuspension in a method of preparing a frozen formulation comprises sodium acetate (NaCH₃COO, also abbreviated NaOAc). In some embodiments, the isotonic crystalloid solution used for resuspension in a method of preparing a frozen formulation comprises sodium gluconate (NaC₆H₁₁O₇). In some embodiments, the isotonic crystalloid solution used for resuspension in a method of preparing a frozen formulation comprises a combination of sodium chloride (NaCl), potassium chloride (KCl), magnesium chloride (MgCl₂), sodium acetate (NaCH₃COO, also abbreviated NaOAc), and sodium gluconate (NaC₆H₁₁O₇).

In some embodiments, the isotonic crystalloid solution used for resuspension in a method of preparing a frozen formulation comprises 140 mmol/L sodium, 5 mmol/L potassium, 1.5 mmol/L magnesium, 98 mmol/L chloride, 27 mmol/L acetate, and 23 mmol/L gluconate. In some embodiments, the isotonic crystalloid solution used for resuspension in a method of preparing a frozen formulation comprises PLASMA-LYTE 148 (Baxter USA), also known as Normasol® (Molnlycke Sweden).

In some embodiments, the isotonic crystalloid solution used for resuspension in a method of preparing a frozen formulation comprises a normal saline solution (0.9% NaCl solution), a lactated Ringer's/Hartman's solution (lactate buffered solution), an acetate buffered solution, an acetate and lactate buffered solution, an acetate and gluconate buffered solution, 5% dextrose in water, or 10% dextrose in water, or any combination thereof. In some embodiments, the isotonic crystalloid solution used for resuspension in a method of preparing a frozen formulation comprises a normal saline solution (0.9% NaCl solution). In some embodiments, the isotonic crystalloid solution used for resuspension in a method of preparing a frozen formulation comprises a lactated Ringer's/Hartman's solution (lactate buffered solution). In some embodiments, the isotonic crystalloid solution used for resuspension in a method of preparing a frozen formulation comprises an acetate buffered solution. In some embodiments, the isotonic crystalloid solution used for resuspension in a method of preparing a frozen formulation comprises an acetate and lactate buffered solution. In some embodiments, the isotonic crystalloid solution used for resuspension in a method of preparing a frozen formulation comprises an acetate and gluconate buffered solution. In some embodiments, the isotonic crystalloid solution used for resuspension in a method of preparing a frozen formulation comprises 5% dextrose in water. In some embodiments, the isotonic crystalloid solution used for resuspension in a method of preparing a frozen formulation comprises 10% dextrose in water.

In some embodiments, the isotonic crystalloid solution used in methods of preparing a frozen formulation of apoptotic mononuclear cells is serum free. In some embodiments, the isotonic crystalloid solution used in methods of preparing a frozen formulation of apoptotic mononuclear cells is protein free. In some embodiments, the isotonic crystalloid solution used in methods of preparing a frozen formulation of apoptotic mononuclear cells is calcium (Ca++) free. In some embodiments, the isotonic crystalloid solution used in methods of preparing a frozen formulation of apoptotic mononuclear cells does not contain antimicrobial agents.

In some embodiments, the isotonic crystalloid solution used in methods of preparing a frozen formulation of apoptotic mononuclear cells has a pH of about pH 6.5-8.0. In some embodiments, the isotonic crystalloid solution used for resuspension in a method of preparing a frozen formulation has a pH of about 7.0-8.0. In some embodiments, the isotonic crystalloid solution used for resuspension in a method of preparing a frozen formulation has a pH of about pH 6.5-7.5. In some embodiments, the isotonic crystalloid solution used for resuspension in a method of preparing a frozen formulation has a pH of about pH 7.0-7.5. In some embodiments, the isotonic crystalloid solution used for resuspension in a method of preparing a frozen formulation has a pH of about pH 7.5-8.0. In some embodiments, the isotonic crystalloid solution used for resuspension in a method of preparing a frozen formulation has a pH of about pH 6.5, 6.6, 6.7, 6.8, 6.9, 7.0, 7.1, 7.2, 7.3, 7.4, 7.5, 7.6, 7.7, 7.8, 7.9, or 8.0. In some embodiments, the pH of a frozen formulation comprising apoptotic mononuclear enriched cells is pH 6.5, 6.6, 6.7, 6.8, 6.9, 7.0, 7.1, 7.2, 7.3, 7.4, 7.5, 7.6, 7.7, 7.8, 7.9, or 8.0.

In some embodiments, the isotonic crystalloid solution used for resuspension in a method of preparing a frozen formulation has a pH of about 6.5. In some embodiments, the isotonic crystalloid solution used for resuspension in a method of preparing a frozen formulation has a pH of about 6.6. In some embodiments, the isotonic crystalloid solution used for resuspension in a method of preparing a frozen formulation has a pH of about 6.7. In some embodiments, the isotonic crystalloid solution used for resuspension in a method of preparing a frozen formulation has a pH of about 6.8. In some embodiments, the isotonic crystalloid solution used for resuspension in a method of preparing a frozen formulation has a pH of about 6.9. In some embodiments, the isotonic crystalloid solution used for resuspension in a method of preparing a frozen formulation has a pH of about 7.0. In some embodiments, the isotonic crystalloid solution used for resuspension in a method of preparing a frozen formulation has a pH of about 7.1. In some embodiments, the isotonic crystalloid solution used for resuspension in a method of preparing a frozen formulation has a pH of about 7.2. In some embodiments, the isotonic crystalloid solution used for resuspension in a method of preparing a frozen formulation has a pH of about 7.3. In some embodiments, the isotonic crystalloid solution used for resuspension in a method of preparing a frozen formulation has a pH of about 7.4. In some embodiments, the isotonic crystalloid solution used for resuspension in a method of preparing a frozen formulation has a pH of about 7.5. In some embodiments, the isotonic crystalloid solution used for resuspension in a method of preparing a frozen formulation has a pH of about 7.6. In some embodiments, the isotonic crystalloid solution used for resuspension in a method of preparing a frozen formulation has a pH of about 7.7. In some embodiments, the isotonic crystalloid solution used for resuspension in a method of preparing a frozen formulation has a pH of about 7.8. In some embodiments, the isotonic crystalloid solution used for resuspension in a method of preparing a frozen formulation has a pH of about 7.9. In some embodiments, the isotonic crystalloid solution used for resuspension in a method of preparing a frozen formulation has a pH of about 8.0.

In some embodiments, the isotonic crystalloid solution used for resuspension in a method of preparing a frozen formulation has a pH of 6.5. In some embodiments, the isotonic crystalloid solution used for resuspension in a method of preparing a frozen formulation has a pH of 6.6. In some embodiments, the isotonic crystalloid solution used for resuspension in a method of preparing a frozen formulation has a pH of 6.7. In some embodiments, the isotonic crystalloid solution used for resuspension in a method of preparing a frozen formulation has a pH of 6.8. In some embodiments, the isotonic crystalloid solution used for resuspension in a method of preparing a frozen formulation has a pH of 6.9. In some embodiments, the isotonic crystalloid solution used for resuspension in a method of preparing a frozen formulation has a pH of 7.0. In some embodiments, the isotonic crystalloid solution used for resuspension in a method of preparing a frozen formulation has a pH of 7.1. In some embodiments, the isotonic crystalloid solution used for resuspension in a method of preparing a frozen formulation has a pH of 7.2. In some embodiments, the isotonic crystalloid solution used for resuspension in a method of preparing a frozen formulation has a pH of 7.3. In some embodiments, the isotonic crystalloid solution used for resuspension in a method of preparing a frozen formulation has a pH of 7.4. In some embodiments, the isotonic crystalloid solution used for resuspension in a method of preparing a frozen formulation has a pH of 7.5. In some embodiments, the isotonic crystalloid solution used for resuspension in a method of preparing a frozen formulation has a pH of 7.6. In some embodiments, the isotonic crystalloid solution used for resuspension in a method of preparing a frozen formulation has a pH of 7.7. In some embodiments, the isotonic crystalloid solution used for resuspension in a method of preparing a frozen formulation has a pH of 7.8. In some embodiments, the isotonic crystalloid solution used for resuspension in a method of preparing a frozen formulation has a pH of 7.9. In some embodiments, the isotonic crystalloid solution used for resuspension in a method of preparing a frozen formulation has a pH of 8.0.

In some embodiments of a method of preparing a frozen formulation of apoptotic mononuclear enriched cells, the step of resuspension in an isotonic crystalloid solution comprises a volume reduction. For example, the volume reduction and media exchange steps may be performed by the LOVO cell processing system (Fresenius Kabi, USA). The LOVO is an automated closed-kit cell washing and processing device that pumps a cell solution through a spinning membrane filtration device to allow for rapid fluid management and fast, efficient cell processing. A LOVO procedure consists of a series of wash cycles which include volume reductions, followed by cell resuspension in a new, configurable solution. The procedure results in a final product containing the original cells, with the newly selected solution. The use of the spinning membrane technology results in a fast continuous flow and nonpelletizing cell processing.

In some embodiments, the step of resuspending including volume reduction results in an apoptotic mononuclear enriched cell population comprising a concentration of cells of about 10×10\hat{ }6-150×10\hat{ }⁶ cells/ml. In some embodiments, the step of resuspending including volume reduction results in an apoptotic mononuclear enriched cell population comprising a concentration of cells of about 50-80 million cells/ml. In some embodiments, the step of resuspending including volume reduction results in an apoptotic mononuclear enriched cell population comprising a concentration of cells of about 20×10\hat{ }⁶-150×10\hat{ }⁶ cells/ml. In some embodiments, the step of resuspending including volume reduction results in an apoptotic mononuclear enriched cell population comprising a concentration of cells of about 20×10\hat{ }⁶-100×10\hat{ }⁶ cells/ml. In some embodiments, the step of resuspending including volume reduction results in an apoptotic mononuclear enriched cell population comprising a concentration of cells of about 25×10\hat{ }⁶-100×10\hat{ }⁶ cells/ml. In some embodiments, the step of resuspending including volume reduction results in an apoptotic mononuclear enriched cell population comprising a concentration of cells of about 25×10\hat{ }⁶-80×10\hat{ }⁶ cells/ml. In some embodiments, the step of resuspending including volume reduction results in an apoptotic mononuclear enriched cell population comprising a concentration of cells of about 25×10\hat{ }⁶-60×10\hat{ }⁶ cells/ml. In some embodiments, the step of resuspending including volume reduction results in an apoptotic mononuclear enriched cell population comprising a concentration of cells of about 25×10\hat{ }⁶-40×10\hat{ }⁶ cells/ml. In some embodiments, the step of resuspending including volume reduction results in an apoptotic mononuclear enriched cell population comprising a concentration of cells of about 20×10\hat{ }⁶-60×10\hat{ }⁶ cells/ml. In some embodiments, the step of resuspending including volume reduction results in an apoptotic mononuclear enriched cell population comprising a concentration of cells of about 25×10\hat{ }⁶-50×10\hat{ }⁶ cells/ml. In some embodiments, the step of resuspending including volume reduction results in an apoptotic mononuclear enriched cell population comprising a concentration of cells of about 25×10\hat{ }⁶-40×10\hat{ }⁶ cells/ml. In some embodiments, the step of resuspending including volume reduction results in an apoptotic mononuclear enriched cell population comprising a concentration of cells of about 44×10\hat{ }⁶-50×10\hat{ }⁶ cells/ml. In some embodiments, the step of resuspending including volume reduction results in an apoptotic mononuclear enriched cell population comprising a concentration of cells of about 24×10\hat{ }⁶-35×10\hat{ }⁶ cells/ml. In some embodiments, the step of resuspending including volume reduction results in an apoptotic mononuclear enriched cell population comprising a concentration of cells of about 20×10\hat{ }⁶-40×10\hat{ }⁶ cells/ml. In some embodiments, the step of resuspending including volume reduction results in an apoptotic mononuclear enriched cell population comprising a concentration of cells of about 20×10\hat{ }⁶-35×10\hat{ }⁶ cells/ml. In some embodiments, the step of resuspending including volume reduction results in an apoptotic mononuclear enriched cell population comprising a concentration of cells of about 25×10\hat{ }⁶-35×10\hat{ }⁶ cells/ml. In some embodiments, the step of resuspending including volume reduction results in an apoptotic mononuclear enriched cell population comprising a concentration of cells of about 35×10\hat{ }⁶-45×10\hat{ }⁶ cells/ml. In some embodiments, the step of resuspending including volume reduction results in an apoptotic mononuclear enriched cell population comprising a concentration of cells of about 35×10\hat{ }⁶-40×10\hat{ }⁶ cells/ml. In some embodiments, the step of resuspending including volume reduction results in an apoptotic mononuclear enriched cell population comprising a concentration of cells of about 40×10\hat{ }⁶-50×10\hat{ }⁶ cells/ml. In some embodiments, the step of resuspending including volume reduction results in an apoptotic mononuclear enriched cell population comprising a concentration of cells of about 40×10\hat{ }⁶-60×10\hat{ }⁶ cells/ml.

In some embodiments, the step of resuspending including volume reduction results in an apoptotic mononuclear enriched cell population comprising 10×10\hat{ }⁹±20% cells at the time of freezing. In some embodiments, the step of resuspending including volume reduction results in an apoptotic mononuclear enriched cell population comprising 8×10\hat{ }⁹-12×10\hat{ }⁹±20% cells at the time of freezing. In some embodiments, the step of resuspending including volume reduction results in an apoptotic mononuclear enriched cell population comprising 10×10\hat{ }⁹±20% cells at the time of freezing. In some embodiments, the step of resuspending including volume reduction results in an apoptotic mononuclear enriched cell population comprising 8×10\hat{ }⁹-12×10\hat{ }⁹±20% cells at the time of freezing. In some embodiments, the step of resuspending including volume reduction results in an apoptotic mononuclear enriched cell population comprising 8×10\hat{ }⁹±20% cells at the time of freezing. In some embodiments, the step of resuspending including volume reduction results in an apoptotic mononuclear enriched cell population comprising 9×10\hat{ }⁹±20% cells at the time of freezing. In some embodiments, the step of resuspending including volume reduction results in an apoptotic mononuclear enriched cell population comprising 10×10\hat{ }⁹±20% cells at the time of freezing. In some embodiments, the step of resuspending including volume reduction results in an apoptotic mononuclear enriched cell population comprising 11×10\hat{ }⁹±20% cells at the time of freezing. In some embodiments, the step of resuspending including volume reduction results in an apoptotic mononuclear enriched cell population comprising 12×10\hat{ }⁹±20% cells at the time of freezing.

In some embodiments, following resuspension the resuspended apoptotic mononuclear enriched cells are mixed with a cryoprotectant agent. In some embodiments, the mixing of the resuspended apoptotic mononuclear enriched cells with the cryoprotectant agent is 1:1 (vol/vol). In some embodiments, the mixing of the resuspended apoptotic mononuclear enriched cells with the cryoprotectant agent is at a ratio that yields a % cryoprotectant agent of between about 2.0%-15% (vol/vol). In some embodiments, the mixing of the resuspended apoptotic mononuclear enriched cells with the cryoprotectant agent is at a ratio that yields a % cryoprotectant agent of between about 2.5%-10% (vol/vol). In some embodiments, the mixing of the resuspended apoptotic mononuclear enriched cells with the cryoprotectant agent is at a ratio that yields a % cryoprotectant agent of between about 2.5%-5% (vol/vol). In some embodiments, the mixing of the resuspended apoptotic mononuclear enriched cells with the cryoprotectant agent is at a ratio that yields a % cryoprotectant agent of between about 5%-10% (vol/vol). In some embodiments, the mixing of the resuspended apoptotic mononuclear enriched cells with the cryoprotectant agent is at a ratio that yields a % cryoprotectant agent of between about 5%-15% (vol/vol).

In some embodiments, the mixing of the resuspended apoptotic mononuclear enriched cells with the cryoprotectant agent is at a ratio that yields a % cryoprotectant agent of less than 2.0% (vol/vol). In some embodiments, the mixing of the resuspended apoptotic mononuclear enriched cells with the cryoprotectant agent is at a ratio that yields a % cryoprotectant agent of less than 2.5% (vol/vol). In some embodiments, the mixing of the resuspended apoptotic mononuclear enriched cells with the cryoprotectant agent is at a ratio that yields a % cryoprotectant agent of less than 3.0% (vol/vol). In some embodiments, the mixing of the resuspended apoptotic mononuclear enriched cells with the cryoprotectant agent is at a ratio that yields a % cryoprotectant agent of less than 3.5% (vol/vol). In some embodiments, the mixing of the resuspended apoptotic mononuclear enriched cells with the cryoprotectant agent is at a ratio that yields a % cryoprotectant agent of less than 4.0% (vol/vol). In some embodiments, the mixing of the resuspended apoptotic mononuclear enriched cells with the cryoprotectant agent is at a ratio that yields a % cryoprotectant agent of less than 4.5% (vol/vol). In some embodiments, the mixing of the resuspended apoptotic mononuclear enriched cells with the cryoprotectant agent is at a ratio that yields a % cryoprotectant agent of less than 5.0% (vol/vol). In some embodiments, the mixing of the resuspended apoptotic mononuclear enriched cells with the cryoprotectant agent is at a ratio that yields a % cryoprotectant agent of less than 5.5% (vol/vol). In some embodiments, the mixing of the resuspended apoptotic mononuclear enriched cells with the cryoprotectant agent is at a ratio that yields a % cryoprotectant agent of less than 6.0% (vol/vol). In some embodiments, the mixing of the resuspended apoptotic mononuclear enriched cells with the cryoprotectant agent is at a ratio that yields a % cryoprotectant agent of less than 6.5% (vol/vol). In some embodiments, the mixing of the resuspended apoptotic mononuclear enriched cells with the cryoprotectant agent is at a ratio that yields a % cryoprotectant agent of less than 7.0% (vol/vol). In some embodiments, the mixing of the resuspended apoptotic mononuclear enriched cells with the cryoprotectant agent is at a ratio that yields a % cryoprotectant agent of less than 7.5% (vol/vol). In some embodiments, the mixing of the resuspended apoptotic mononuclear enriched cells with the cryoprotectant agent is at a ratio that yields a % cryoprotectant agent of less than 8.0% (vol/vol). In some embodiments, the mixing of the resuspended apoptotic mononuclear enriched cells with the cryoprotectant agent is at a ratio that yields a % cryoprotectant agent of less than 8.5% (vol/vol). In some embodiments, the mixing of the resuspended apoptotic mononuclear enriched cells with the cryoprotectant agent is at a ratio that yields a % cryoprotectant agent of less than 9.0% (vol/vol). In some embodiments, the mixing of the resuspended apoptotic mononuclear enriched cells with the cryoprotectant agent is at a ratio that yields a % cryoprotectant agent of less than 9.5% (vol/vol). In some embodiments, the mixing of the resuspended apoptotic mononuclear enriched cells with the cryoprotectant agent is at a ratio that yields a % cryoprotectant agent of less than 10% (vol/vol).

In some embodiments, the mixing of the resuspended apoptotic mononuclear enriched cells with the cryoprotectant agent is at a ratio that yields a % cryoprotectant agent of equal to or less than about 2.0% (vol/vol). In some embodiments, the mixing of the resuspended apoptotic mononuclear enriched cells with the cryoprotectant agent is at a ratio that yields a % cryoprotectant agent of equal to or less than about 2.5% (vol/vol). In some embodiments, the mixing of the resuspended apoptotic mononuclear enriched cells with the cryoprotectant agent is at a ratio that yields a % cryoprotectant agent of equal to or less than about 3.0% (vol/vol). In some embodiments, the mixing of the resuspended apoptotic mononuclear enriched cells with the cryoprotectant agent is at a ratio that yields a % cryoprotectant agent of equal to or less than about 3.5% (vol/vol). In some embodiments, the mixing of the resuspended apoptotic mononuclear enriched cells with the cryoprotectant agent is at a ratio that yields a % cryoprotectant agent of equal to or less than about 4.0% (vol/vol). In some embodiments, the mixing of the resuspended apoptotic mononuclear enriched cells with the cryoprotectant agent is at a ratio that yields a % cryoprotectant agent of equal to or less than about 4.5% (vol/vol). In some embodiments, the mixing of the resuspended apoptotic mononuclear enriched cells with the cryoprotectant agent is at a ratio that yields a % cryoprotectant agent of equal to or less than about 5.0% (vol/vol). In some embodiments, the mixing of the resuspended apoptotic mononuclear enriched cells with the cryoprotectant agent is at a ratio that yields a % cryoprotectant agent of equal to or less than about 5.5% (vol/vol). In some embodiments, the mixing of the resuspended apoptotic mononuclear enriched cells with the cryoprotectant agent is at a ratio that yields a % cryoprotectant agent of equal to or less than about 6.0% (vol/vol). In some embodiments, the mixing of the resuspended apoptotic mononuclear enriched cells with the cryoprotectant agent is at a ratio that yields a % cryoprotectant agent of equal to or less than about 6.5% (vol/vol). In some embodiments, the mixing of the resuspended apoptotic mononuclear enriched cells with the cryoprotectant agent is at a ratio that yields a % cryoprotectant agent of equal to or less than about 7.0% (vol/vol). In some embodiments, the mixing of the resuspended apoptotic mononuclear enriched cells with the cryoprotectant agent is at a ratio that yields a % cryoprotectant agent of equal to or less than about 7.5% (vol/vol). In some embodiments, the mixing of the resuspended apoptotic mononuclear enriched cells with the cryoprotectant agent is at a ratio that yields a % cryoprotectant agent of equal to or less than about 8.0% (vol/vol). In some embodiments, the mixing of the resuspended apoptotic mononuclear enriched cells with the cryoprotectant agent is at a ratio that yields a % cryoprotectant agent of equal to or less than about 8.5% (vol/vol). In some embodiments, the mixing of the resuspended apoptotic mononuclear enriched cells with the cryoprotectant agent is at a ratio that yields a % cryoprotectant agent of equal to or less than about 9.0% (vol/vol). In some embodiments, the mixing of the resuspended apoptotic mononuclear enriched cells with the cryoprotectant agent is at a ratio that yields a % cryoprotectant agent of equal to or less than about 9.5% (vol/vol). In some embodiments, the mixing of the resuspended apoptotic mononuclear enriched cells with the cryoprotectant agent is at a ratio that yields a % cryoprotectant agent of equal to or less than about 10% (vol/vol).

In some embodiments, the mixing of the resuspended apoptotic mononuclear enriched cells with the cryoprotectant agent is at a ratio that yields a % cryoprotectant agent of about 2.0% (vol/vol). In some embodiments, the mixing of the resuspended apoptotic mononuclear enriched cells with the cryoprotectant agent is at a ratio that yields a % cryoprotectant agent of about 2.5% (vol/vol). In some embodiments, the mixing of the resuspended apoptotic mononuclear enriched cells with the cryoprotectant agent is at a ratio that yields a % cryoprotectant agent of about 3.0% (vol/vol). In some embodiments, the mixing of the resuspended apoptotic mononuclear enriched cells with the cryoprotectant agent is at a ratio that yields a % cryoprotectant agent of about 3.5% (vol/vol). In some embodiments, the mixing of the resuspended apoptotic mononuclear enriched cells with the cryoprotectant agent is at a ratio that yields a % cryoprotectant agent of about 4.0% (vol/vol). In some embodiments, the mixing of the resuspended apoptotic mononuclear enriched cells with the cryoprotectant agent is at a ratio that yields a % cryoprotectant agent of about 4.5% (vol/vol). In some embodiments, the mixing of the resuspended apoptotic mononuclear enriched cells with the cryoprotectant agent is at a ratio that yields a % cryoprotectant agent of about 5.0% (vol/vol). In some embodiments, the mixing of the resuspended apoptotic mononuclear enriched cells with the cryoprotectant agent is at a ratio that yields a % cryoprotectant agent of about 5.5% (vol/vol). In some embodiments, the mixing of the resuspended apoptotic mononuclear enriched cells with the cryoprotectant agent is at a ratio that yields a % cryoprotectant agent of about 6.0% (vol/vol). In some embodiments, the mixing of the resuspended apoptotic mononuclear enriched cells with the cryoprotectant agent is at a ratio that yields a % cryoprotectant agent of about 6.5% (vol/vol). In some embodiments, the mixing of the resuspended apoptotic mononuclear enriched cells with the cryoprotectant agent is at a ratio that yields a % cryoprotectant agent of about 7.0% (vol/vol). In some embodiments, the mixing of the resuspended apoptotic mononuclear enriched cells with the cryoprotectant agent is at a ratio that yields a % cryoprotectant agent of about 7.5% (vol/vol). In some embodiments, the mixing of the resuspended apoptotic mononuclear enriched cells with the cryoprotectant agent is at a ratio that yields a % cryoprotectant agent of about 8.0% (vol/vol). In some embodiments, the mixing of the resuspended apoptotic mononuclear enriched cells with the cryoprotectant agent is at a ratio that yields a % cryoprotectant agent of about 8.5% (vol/vol). In some embodiments, the mixing of the resuspended apoptotic mononuclear enriched cells with the cryoprotectant agent is at a ratio that yields a % cryoprotectant agent of about 9.0% (vol/vol). In some embodiments, the mixing of the resuspended apoptotic mononuclear enriched cells with the cryoprotectant agent is at a ratio that yields a % cryoprotectant agent of about 9.5% (vol/vol). In some embodiments, the mixing of the resuspended apoptotic mononuclear enriched cells with the cryoprotectant agent is at a ratio that yields a % cryoprotectant agent of about 10% (vol/vol).

In some embodiments, the cryoprotectant agent used in the method of preparing a frozen formulation of apoptotic mononuclear enriched cells comprises glycerol, ethylene glycol, propylene glycol, or dimethyl sulfoxide (DMSO). In some embodiments, the cryoprotectant agent used in the method of preparing a frozen formulation of apoptotic mononuclear enriched cells comprises glycerol. In some embodiments, the cryoprotectant agent used in the method of preparing a frozen formulation of apoptotic mononuclear enriched cells comprises ethylene glycol. In some embodiments, the cryoprotectant agent used in the method of preparing a frozen formulation of apoptotic mononuclear enriched cells comprises propylene glycol. In some embodiments, the cryoprotectant agent used in the method of preparing a frozen formulation of apoptotic mononuclear enriched cells comprises dimethyl sulfoxide (DMSO). In some embodiments, the cryoprotectant agent used in the method of preparing a frozen formulation of apoptotic mononuclear enriched cells comprises a cryoprotectant agent that is comprised within solution.

In some embodiments, when the cryoprotectant agent comprises DMSO, the step of mixing of the resuspended apoptotic mononuclear enriched cells with the cryoprotectant agent is at a ratio that yields a % DMSO of between about 2%-10% DMSO (vol/vol). In some embodiments, the step of mixing of the resuspended apoptotic mononuclear enriched cells with a cryoprotectant agent comprising DMSO is at a ratio that yields a % DMSO of between about 2.5%-10% DMSO (vol/vol). In some embodiments, the step of mixing of the resuspended apoptotic mononuclear enriched cells with a cryoprotectant agent comprising DMSO is at a ratio that yields a % DMSO of between about 2.0%-5% DMSO (vol/vol). In some embodiments, the step of mixing of the resuspended apoptotic mononuclear enriched cells with a cryoprotectant agent comprising DMSO is at a ratio that yields a % DMSO of between about 2.5%-5% DMSO (vol/vol). In some embodiments, the step of mixing of the resuspended apoptotic mononuclear enriched cells with a cryoprotectant agent comprising DMSO is at a ratio that yields a % DMSO of between about 5%-10% DMSO (vol/vol).

In some embodiments, the step of mixing of the resuspended apoptotic mononuclear enriched cells with a cryoprotectant agent comprising DMSO is at a ratio that yields a % DMSO of less than 10% DMSO (vol/vol). In some embodiments, the step of mixing of the resuspended apoptotic mononuclear enriched cells with a cryoprotectant agent comprising DMSO is at a ratio that yields a % DMSO of less than 9.5%, 8.0%, 8.5%, 7.0%, 7.5%, 6.0%, 6.5%, 5.0%, 5.5%, 4.0%, 4.5%, 3.0%, 3.5%, 2.0% or 2.5% DMSO (vol/vol).

In some embodiments, the step of mixing of the resuspended apoptotic mononuclear enriched cells with a cryoprotectant agent comprising DMSO is at a ratio that yields a % DMSO of less than or equal to 10% DMSO (vol/vol). In some embodiments, the step of mixing of the resuspended apoptotic mononuclear enriched cells with a cryoprotectant agent comprising DMSO is at a ratio that yields a % DMSO of less than or equal to 9.5%, 8.0%, 8.5%, 7.0%, 7.5%, 6.0%, 6.5%, 5.0%, 5.5%, 4.0%, 4.5%, 3.0%, 3.5%, 2.0% or 2.5% DMSO (vol/vol).

In some embodiments, the step of mixing of the resuspended apoptotic mononuclear enriched cells with a cryoprotectant agent comprising DMSO is at a ratio that yields a % DMSO of 2.5% DMSO (vol/vol) or less than 2.5% DMSO (vol/vol). One skilled in the art would appreciate that while DMSO has been recited to demonstrate the different percent concentrations of cryoprotectant, that other cryoprotectants could be used in its place to yield the given percent cryoprotectant.

FIG. 2 shows an embodiment of the methods for making fresh apoptotic mononuclear enriched cell populations and making frozen (cryopreserved) apoptotic mononuclear enriched cell populations, wherein the apoptotic mononuclear enriched cell population, referred to in the figure as Allocetra OTS DP (Allocetra Off the shelf Drug Product), may be frozen in a number of bags designed for this purpose. In other embodiments, a frozen Allocetra product (apoptotic mononuclear enriched cell population) may be cryopreserved in 4 bags of 100 ml each (the total cryopreserved volume would be 400 ml). In some embodiments, the frozen apoptotic mononuclear enrich cell formulation comprises a concentration of between about 10×10\hat{ }6-250×10\hat{ }6 cells/ml±20%. In some embodiments, the frozen apoptotic mononuclear enrich cell formulation comprises a concentration of between about 20×10\hat{ }6-250×10\hat{ }6 cells/ml±20%. In some embodiments, the frozen apoptotic mononuclear enrich cell formulation comprises a concentration of between about 20×10\hat{ }6-300×10\hat{ }6 cells/ml±20%. In some embodiments, the frozen apoptotic mononuclear enrich cell formulation comprises a concentration of between about 25×10\hat{ }6-300×10\hat{ }6 cells/ml±20%. In some embodiments, the frozen apoptotic mononuclear enrich cell formulation comprises a concentration of about 25×10\hat{ }6/ml±20%. In some embodiments, the frozen apoptotic mononuclear enrich cell formulation comprises a concentration of between about 20×10\hat{ }6-30×10\hat{ }6 cells/ml±20%. In some embodiments, the frozen apoptotic mononuclear enrich cell formulation comprises a concentration of about 25-40×10\hat{ }6/ml±20%. In some embodiments, the frozen apoptotic mononuclear enrich cell formulation comprises a concentration of about 25-60×10\hat{ }6/ml±20%. In some embodiments, the frozen apoptotic mononuclear enrich cell formulation comprises a concentration of about 25-80×10\hat{ }6/ml±20%. In some embodiments, the frozen apoptotic mononuclear enrich cell formulation comprises a concentration of about 25-100×10\hat{ }6/ml±20%. In some embodiments, the frozen apoptotic mononuclear enrich cell formulation comprises a concentration of about 25-125×10\hat{ }6/ml±20%. In some embodiments, the frozen apoptotic mononuclear enrich cell formulation comprises a concentration of about 25-150×10\hat{ }6/ml±20%.

In some embodiments, the frozen apoptotic mononuclear enrich cell formulation comprises 10×10\hat{ }9 cells±20%. In some embodiments, the frozen apoptotic mononuclear enrich cell formulation comprises 8-12×10\hat{ }9±20% cells. In some embodiments, the frozen apoptotic mononuclear enrich cell formulation comprises 8×10\hat{ }9±20% cells. In some embodiments, the frozen apoptotic mononuclear enrich cell formulation comprises 9×10\hat{ }9±20% cells. In some embodiments, the frozen apoptotic mononuclear enrich cell formulation comprises 10×10\hat{ }9±20% cells. In some embodiments, the frozen apoptotic mononuclear enrich cell formulation comprises 11×10\hat{ }9±20% cells. In some embodiments, the frozen apoptotic mononuclear enrich cell formulation comprises 12×10\hat{ }9±20% cells.

During methods of preparing a frozen formulation of apoptotic mononuclear enriched cells the cooling rate of the apoptotic cells resuspended in an isotonic crystalloid solution and comprising a cryoprotectant agent may influence the viability and function of the apoptotic cells at the time of thawing. Further, the length of storage of the final temperature of −196° may also influence the viability and function of the apoptotic cells at the time of thawing.

In some embodiments of a method of preparing a frozen formulation of apoptotic mononuclear enriched cells, said gradual freezing step comprises freezing steps at lower and lower temperature to reach a temperature of about −196° C. For example, in some embodiments, immediately after the addition of the cells to bags, the bags may be placed in a−20° C. freezer for 2 hours (in pre-cooled aluminum cassettes), after which they would be transferred to the −80° C. freezer for 2 hours, before being transferred to the liquid nitrogen tank for prolonged storage. Thus, one skilled in the art would appreciate that gradually freezing the apoptotic formulation comprises a stepwise freezing process lowering the temperature with each step, starting with −20° C., to −80° C., to −196° C.

In certain embodiments, some viable non-apoptotic cells (apoptosis resistant) may remain following the induction of apoptosis step (FIG. 2). In some embodiments, the presence of these viable non-apoptotic cells is observed prior to an irradiation step. These viable non-apoptotic cells may be able to proliferate or be activated. In some embodiments, a pooled mononuclear apoptotic cell preparation derived from multiple donors and including viable non-apoptotic cells may be activated against the host, activated against one another, or both. Thus, as shown in FIG. 2, prior to the step of adding a cryoprotectant agent, in some embodiments, the cells may be irradiated. Irradiation of the cell preparation is considered safe in the art. Irradiation procedures are currently performed on a routine basis to donated blood to prevent reactions to WBC.

Methods of preparing a frozen formulation of apoptotic mononuclear-enriched cells, may in certain embodiments include a step of irradiation of the apoptotic cell population following resuspension of the cells in the isotonic crystalloid solution.

In some embodiments, a method of preparing a frozen formulation that includes irradiating the cell population has a population of cells comprising suppressed cellular activation and reduced proliferation compared with a non-irradiated cell preparation. In another embodiment, irradiation used in a step of preparing a frozen formulation disclosed herein, comprises gamma irradiation or UV irradiation. In some embodiments, a method of preparing a frozen formulation that includes irradiating the cell population has a population of cells comprising a reduced number of non-apoptotic cells compared with a non-irradiated cell preparation. In another embodiment, irradiation used in a step of preparing a frozen formulation disclosed herein, comprises about 15 Grey units (Gy). In another embodiment, irradiation used in a step of preparing a frozen formulation disclosed herein, comprises about 20 Grey units (Gy). In another embodiment, irradiation used in a step of preparing a frozen formulation disclosed herein, comprises about 25 Grey units (Gy). In another embodiment, irradiation used in a step of preparing a frozen formulation disclosed herein, comprises about 30 Grey units (Gy). In another embodiment, irradiation used in a step of preparing a frozen formulation disclosed herein, comprises about 35 Grey units (Gy). In another embodiment, irradiation used in a step of preparing a frozen formulation disclosed herein, comprises about 40 Grey units (Gy). In another embodiment, irradiation used in a step of preparing a frozen formulation disclosed herein, comprises about 45 Grey units (Gy). In another embodiment, irradiation used in a step of preparing a frozen formulation disclosed herein, comprises about 50 Grey units (Gy). In another embodiment, irradiation used in a step of preparing a frozen formulation disclosed herein, comprises about 55 Grey units (Gy). In another embodiment, irradiation used in a step of preparing a frozen formulation, comprises about 60 Grey units (Gy). In another embodiment, irradiation used in a step of preparing a frozen formulation disclosed herein, comprises about 65 Grey units (Gy). In another embodiment, irradiation used in a step of preparing a frozen formulation disclosed herein, comprises up to 2500 Gy. In another embodiment, an irradiated pooled apoptotic cell preparation maintains the same or a similar apoptotic profile, stability and efficacy as a non-irradiated pooled apoptotic cell preparation.

In some embodiments of a method of preparing a frozen formulation of apoptotic cells, irradiation comprises irradiating the apoptotic cell population in a way that will decrease proliferation and/or activation of residual viable cells within the apoptotic cell population. In some embodiments of a method of preparing a frozen formulation, apoptotic mononuclear enriched are irradiated in a way that reduces the percent of viable non-apoptotic cells in a population. In some embodiments of a method of preparing a frozen formulation, the percent of viable non-apoptotic cells in an inactivated apoptotic cell population is reduced to less than 65% of the population. In some embodiments of a method of preparing a frozen formulation, the percent of viable non-apoptotic cells in an inactivated apoptotic cell population is reduced to less than 40% of the population. In some embodiments of a method of preparing a frozen formulation, the percent of viable non-apoptotic cells in an inactivated apoptotic cell population is reduced to less than 30% of the population. In some embodiments of a method of preparing a frozen formulation, the percent of viable non-apoptotic cells in an inactivated apoptotic cell population is reduced to less than 20% of the population. In some embodiments of a method of preparing a frozen formulation, the percent of viable non-apoptotic cells in an inactivated apoptotic cell population is reduced to less than 10% of the population. In some embodiments of a method of preparing a frozen formulation, the percent of viable non-apoptotic cells in an inactivated apoptotic cell population is reduced to 0% of the population.

In another embodiment of a method of preparing a frozen population, the irradiated apoptotic mononuclear enriched cells preserve all of their apoptotic-, immune modulation-, stability-properties. In another embodiment of a method of preparing a frozen population, the irradiation step uses UV radiation. In another embodiment, the radiation step uses gamma radiation. In another embodiment of a method of preparing a frozen population, the apoptotic cells comprise a decreased percent of living non-apoptotic cells, comprise a preparation having a suppressed cellular activation of any living non-apoptotic cells present within the apoptotic cell preparation, or comprise a preparation having reduced proliferation of any living non-apoptotic cells present within the apoptotic mononuclear enriched cell preparation, or any combination thereof.

In some embodiments of a method of preparing a frozen formulation, irradiation of apoptotic cells does not increase the population of dead cells (secondary necrosis; PI+) compared with apoptotic cells not irradiated. In some embodiments of a method of preparing a frozen formulation, irradiation of apoptotic cells does not increase the population of dead cells (secondary necrosis; PI+) by more than about 1% compared with apoptotic cells not irradiated. In some embodiments of a method of preparing a frozen formulation, irradiation of apoptotic cells does not increase the population of dead cells (secondary necrosis; PI+) by more than about 2% compared with apoptotic cells not irradiated. In some embodiments of a method of preparing a frozen formulation, irradiation of apoptotic cells does not increase the population of dead cells (secondary necrosis; PI+) by more than about 3% compared with apoptotic cells not irradiated. In some embodiments of a method of preparing a frozen formulation, irradiation of apoptotic cells does not increase the population of dead cells (secondary necrosis; PI+) by more than about 4% compared with apoptotic cells not irradiated. In some embodiments of a method of preparing a frozen formulation, irradiation of apoptotic cells does not increase the population of dead cells (secondary necrosis; PI+) by more than about 5% compared with apoptotic cells not irradiated. In some embodiments of a method of preparing a frozen formulation, irradiation of apoptotic cells does not increase the population of dead cells (secondary necrosis; PI+) by more than about 6% compared with apoptotic cells not irradiated. In some embodiments of a method of preparing a frozen formulation, irradiation of apoptotic cells does not increase the population of dead cells (secondary necrosis; PI+) by more than about 7% compared with apoptotic cells not irradiated. In some embodiments of a method of preparing a frozen formulation, irradiation of apoptotic cells does not increase the population of dead cells (secondary necrosis; PI+) by more than about 8% compared with apoptotic cells not irradiated. In some embodiments of a method of preparing a frozen formulation, irradiation of apoptotic cells does not increase the population of dead cells (secondary necrosis; PI+) by more than about 9% compared with apoptotic cells not irradiated. In some embodiments of a method of preparing a frozen formulation, irradiation of apoptotic cells does not increase the population of dead cells (secondary necrosis; PI+) by more than about 10% compared with apoptotic cells not irradiated. In some embodiments of a method of preparing a frozen formulation, irradiation of apoptotic cells does not increase the population of dead cells (secondary necrosis; PI+) by more than about 15% compared with apoptotic cells not irradiated. In some embodiments of a method of preparing a frozen formulation, irradiation of apoptotic cells does not increase the population of dead cells (secondary necrosis; PI+) by more than about 20%, 25%, 30%, 35%, 40%, 45%, or 50% compared with apoptotic cells not irradiated.

In some embodiments, a method of preparing a frozen formulation of apoptotic mononuclear enriched cells, further comprises a step of irradiation of said apoptotic cell population following said resuspension step and prior to said mixing step with a cryoprotectant.

### Therapeutic Methods of Use of Frozen Formulations

An advantage of frozen populations comprising apoptotic mononuclear enriched cells is the ability to have ready-made a therapeutic population comprising apoptotic cells ready for use treating a subject in need.

A skilled artisan would appreciate that the apoptotic state of freshly prepared frozen apoptotic cells may be the same or may differ slightly from the state of apoptotic cells that have been frozen and thawed. Importantly, the frozen populations of apoptotic mononuclear enriched cells disclosed herein maintain their biologic activities (potency) as exemplified in Example 1.

In certain embodiments, a method of treating a subject in need with a frozen apoptotic formulation as described herein, comprises slowly thawing said frozen formulation; followed by administering said thawed cells to said subject in need.

In some embodiments of a method of treating a subject in need using a frozen formulation described here, a step of thawing the cells comprises thawing the cells by placing a bag or bags containing frozen formulations of apoptotic cells in a pre-heated 37° C. bath and gently agitating until no ice particles remain. In some embodiments of a method of treating a subject in need using a frozen formulation described here, a step of thawing the cells comprises thawing the cells by using a dry thawing method until no ice particles remain.

In some embodiments, a method of treating a subject in need comprises resuspending the thawed apoptotic mononuclear cell population in an administration media formulation for administration. In some embodiments, thawed apoptotic mononuclear cell preps are resuspended in a suitable physiological buffer. In some embodiments, thawed apoptotic mononuclear cell population is thawed and administered, without need of changing the formulation in which the cells were frozen.

In some embodiments, a method of treating a subject in need comprises administering an autologous, thawed apoptotic mononuclear enriched cell population to a subject in need. In some embodiments, a method of treating a subject in need comprises administering an allogeneic thawed apoptotic cell population to a subject in need.

In some embodiments, administering comprises a single infusion of the thawed apoptotic cell population. In some embodiments, administering comprises multiple infusions of thawed apoptotic populations.

In some embodiments, thawed apoptotic cells may be administered as a prophylactic to a subject predetermined to be at risk for a disease, disorder, or condition. In some embodiments, thawed apoptotic cells may be administered as a prophylactic to a subject who is asymptomatic for a disease, disorder, or condition. In some embodiments, thawed apoptotic cells may be administered to a subject experiencing mild, moderate, severe, or critical form of a disease, disorder, or condition. In some embodiments, thawed apoptotic cells may be administered as a prophylactic to an asymptomatic subject in order to prevent, reduce the risk of, or delay the appearance of mild, moderate, severe, or critical symptoms of a disease, disorder, or condition.

In some embodiments, a method of treating a subject in need comprises treating, preventing, ameliorating, inhibiting, or reducing the incidence of an immune disease, an autoimmune disease, a cytokine release syndrome (CRS), a cytokine storm, an inflammatory disease, a cancer, osteoarthritis, vanishing bone disease, an infection, COVID19, ARDS, fibrotic lung disease, or at least one symptom associated with an infection, or a combination thereof, in a subject in need thereof. In some embodiments, methods of treatment herein are for reducing or inhibiting the incidence of CRS or a cytokine storm in a subject.

In some embodiments, methods of use of thawed apoptotic cells comprise treating an immune disease in a subject. In some embodiments, methods of use of thawed apoptotic cells comprise treating an autoimmune disease in a subject. In some embodiments, methods of use of thawed apoptotic cells comprise treating CRS in a subject. In some embodiments, methods of use of thawed apoptotic cells comprise treating a cytokine storm in a subject. In some embodiments, methods of use of thawed apoptotic cells comprise treating an inflammatory disease in a subject. In some embodiments, methods of use of thawed apoptotic cells comprise treating a cancer in a subject. In some embodiments, methods of use of thawed apoptotic cells comprise treating osteoarthritis in a subject. In some embodiments, methods of use of thawed apoptotic cells comprise treating vanishing bone disease in a subject. In some embodiments, methods of use of thawed apoptotic cells comprise treating an infection in a subject. In some embodiments, methods of use of thawed apoptotic cells comprise treating a viral or bacterial infection in a subject. In some embodiments, methods of use of thawed apoptotic cells comprise treating COVID19 in a subject. In some embodiments, methods of use of thawed apoptotic cells comprise treating a subject suffering from ARDS. In some embodiments, methods of use of thawed apoptotic cells comprise treating a subject suffering from fibrotic lung disease. In some embodiments, methods of use of thawed apoptotic cells comprise treating at least one symptom associated with an infection in a subject, wherein said infection may be viral or bacterial. In other embodiments, thawed early apoptotic populations administered for treating an immune disease, an autoimmune disease, a CRS, a cytokine storm, an inflammatory disease, a cancer, osteoarthritis, vanishing bone disease, an infection or at least one symptom associated with an infection, or a combination thereof in a subject, further comprise reducing or inhibiting the incidence of CRS or a cytokine storm.

In some embodiments, a viral, bacterial, or fungal infection causes the cytokine release syndrome or cytokine storm in the subject. In some embodiments, the infection is a SARS-CoV-2 infection. In some embodiments, the infection is an influenza infection. In some embodiments, the influenza infection is H1N1. In other embodiments, the influenza infection is an H5N1 bird flu. In other embodiments, the infection is severe acute respiratory syndrome (SARS). In other embodiments, the subject has Epstein-Barr virus-associated hemophagocytic lymph histiocytosis (HLH). In other embodiments, the infection is sepsis. In some embodiments, the sepsis is caused by a gram-negative bacterium. In other embodiments, the infection is malaria. In other embodiments, the infection is an Ebola virus infection. In other embodiments, the infection is an Epstein-Barr virus-associated infection. In other embodiments, the infection is variola virus infection. In other embodiments, the infection is a systemic Gram-negative bacterial infection. In other embodiments, the infection is Jarisch-Herxheimer syndrome.

In some embodiments, the cause of the cytokine release syndrome or cytokine storm in a subject is hemophagocytic lymph histiocytosis (HLH). In other embodiments, HLH is sporadic HLH. In other embodiments, HLH is macrophage activation syndrome (MAS). In other embodiments, the cause of the cytokine release syndrome or cytokine storm in a subject is MAS.

In some embodiments, the cause of the cytokine release syndrome or cytokine storm in a subject is chronic arthritis. In other embodiments, the cause of the cytokine release syndrome or cytokine storm in a subject is systemic Juvenile Idiopathic Arthritis (sJIA), also known as Still's Disease.

In some embodiments, the cause of the cytokine release syndrome or cytokine storm in a subject is Cryopyrin-associated Periodic Syndrome (CAPS). In other embodiments, CAPS comprises Familial Cold Auto-inflammatory Syndrome (FCAS), also known as Familial Cold Urticaria (FCU). In other embodiments, CAPS comprises Muckle-Well Syndrome (MWS). In other embodiments, CAPS comprises Chronic Infantile Neurological Cutaneous and Articular (CINCA) Syndrome. In yet another embodiment, CAPS comprises FCAS, FCU, MWS, or CINCA Syndrome, or any combination thereof. In other embodiments, the cause of the cytokine release syndrome or cytokine storm in a subject is FCAS. In other embodiments, the cause of the cytokine release syndrome or cytokine storm in a subject is FCU. In other embodiments, the cause of the cytokine release syndrome or cytokine storm in a subject is MWS. In other embodiments, the cause of the cytokine release syndrome or cytokine storm in a subject is CINCA Syndrome. In still another embodiment, the cause of the cytokine release syndrome or cytokine storm in a subject is FCAS, FCU, MWS, or CINCA Syndrome, or any combination thereof.

In other embodiments, the cause of the cytokine release syndrome or cytokine storm in a subject is a cryopyrinopathy comprising inherited or de novo gain of function mutations in the NLRP3 gene, also known as the CIASI gene.

In some embodiments, the cause of the cytokine release syndrome or cytokine storm in a subject is a hereditary auto-inflammatory disorder.

In some embodiments, the trigger for the release of inflammatory cytokines is a lipopolysaccharide (LPS), Gram-positive toxins, fungal toxins, glycosylphosphatidylinositol (GPI) or modulation of RIG-1 gene expression.

In other embodiments, the subject in need has cytokine release syndrome or cytokine storm secondary to receipt of a therapy.

In some embodiments, a method of inhibiting or reducing the incidence of a cytokine release syndrome (CRS) or a cytokine storm in a subject undergoing CAR T-cell cancer therapy comprises administering a thawed apoptotic population as disclosed herein.

In some embodiments, the subject in need is suffering from a disease, disorder, or condition, comprising a cancer. In some embodiments, the cancer comprises bladder cancer, breast cancer, cervical cancer, colorectal cancer, endometrial cancer, head and neck cancer, kidney cancer, leukemia, liver cancer, lung cancer, ovarian cancer, pancreatic cancer, prostate cancer, skin cancer, thyroid cancer, or a combination thereof. In other embodiments, the cancer comprises a carcinoma, a sarcoma, a leukemia, a lymphoma, a myeloma, a mixed cancer type, or a combination thereof. In some embodiments, the lymphoma comprises non-Hodgkin lymphoma. In other embodiments, the cancer comprises a solid tumor or a solid cancer. In some embodiments, the cancer comprises a non-solid tumor. In other embodiments, the cancer comprises a non-solid cancer.

In other embodiments, the subject in need is suffering from a disease, disorder, or condition comprising osteoarthritis. In other embodiments, the subject in need is suffering from a disease, disorder, or condition comprising vanishing bone disease.

In other embodiments, the subject in need is suffering from a disease, disorder, or condition comprising an infectious disease. In some embodiments, the infectious disease comprises Chickenpox, Common cold, Coronavirus, Diphtheria, E. coli, Giardiasis, HIV/AIDS, Infectious mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis), or a combination thereof.

In other embodiments, the disease, disorder, or condition comprises a viral or bacterial infection. In some embodiments, the infection is an influenza infection. In some embodiments, the influenza infection is H1N1. In other embodiments, the influenza infection is an H5N1 bird flu. In other embodiments, the infection is severe acute respiratory syndrome (SARS). In other embodiments, the subject has Epstein-Barr virus-associated hemophagocytic lymph histiocytosis (HLH). In other embodiments, the infection is sepsis. In some embodiments, the sepsis is caused by a gram-negative bacterium. In other embodiments, the infection is malaria. In other embodiments, the infection is an Ebola virus infection. In other embodiments, the infection is a variola virus infection. In other embodiments, the infection is a systemic Gram-negative bacterial infection. In other embodiments, the infection is Jarisch-Herxheimer syndrome. In other embodiments, the infection comprises a SARS infection. In other embodiments, the infection comprises a COVID19 or SARS-COV-2 infection. In other embodiments, the infection comprises a septal infection. In other embodiments, the infection comprises a malarial infection. In other embodiments, the infection comprises an Ebolavirus infection. In other embodiments, the infection comprises an Epstein-Barr virus-associated infection.

In other embodiments, the subject in need is suffering from COVID19.

In some embodiments, methods as described herein are for treating, preventing, ameliorating, inhibiting, or reducing the incidence of COVID19 in a subject. In some embodiments, disclosed herein is a method of treating COVID-19 in a subject infected by SARS-COV-2 virus, said method comprising administering a thawed apoptotic mononuclear enriched population to the subject. In certain embodiments, methods of treating comprise treating, inhibiting, reducing the incidence of, ameliorating, or alleviating a symptom of COVID-19. In certain embodiments, methods of treating comprise preventing the appearance of symptoms of COVID-19. In some embodiments, methods of treating COVID-19 comprising administering a thawed apoptotic cell population that results in a PCR negative result for SARS-COV-2.

In some embodiments, the subject is asymptomatic for COVID19. In other embodiments, the subject has symptoms of COVID19.

In some embodiments, COVID19 symptoms comprise any one or more of the following: organ failure, organ dysfunction, organ damage, a cytokine storm, a chemokine storm, a multisystem inflammatory syndrome, and a cytokine release syndrome. In some embodiments, the organ comprises heart, kidney, liver, or a combination thereof. In other embodiments, COVID19 symptoms comprise one or more hematological aberrations. In some embodiments, the hematological aberrations comprise thrombocytopenia, lymphopenia, neutropenia, or neutrophilia, or any combination thereof. In some embodiments, COVID19 symptoms comprise cardiovascular dysfunction, acute kidney injury, or a combination thereof.

In some embodiments, treating a subject in need suffering from COVID-19 comprises treating a subject suffering from mild COVID-19. In some embodiments, treating a subject in need suffering from COVID-19 comprises treating a subject suffering from moderate COVID-19. In some embodiments, treating a subject in need suffering from COVID-19 comprises treating a subject suffering from severe COVID-19. In some embodiments, treating a subject in need suffering from COVID-19 comprises treating a subject suffering from critical COVID-19.

In other embodiments, a subject in need is suffering from sepsis. In some embodiments, the sepsis comprises severe sepsis. In other embodiments, the sepsis comprises septic shock.

In other embodiments, a subject in need is suffering from GVHD. In some embodiments, the immune disease is GVHD. In some embodiments, the present disclosure provides the pharmaceutical composition for use in treating, preventing, ameliorating, inhibiting, or reducing the incidence of GVHD in a subject in need thereof.

In some embodiments, a method of treating an immune disease in a subject in need comprises administering a thawed apoptotic cell population to a subject undergoing solid organ transplantation. In some embodiments, the organ comprises lung, heart, kidney, pancreas, liver, skin and small-bowel. In other embodiments, a solid organ comprises beta cells. In other embodiments, a solid organ is a limb.

In some embodiments, the subject in need is suffering from a hematopoietic malignancy, retains a graft-versus-tumor or graft-versus-leukemia (GVL) effect, is undergoing hematopoietic stem-cell transplantation (HSCT), is undergoing chimeric antigen receptor-expressing T-cell (CAR T-cell) cancer therapy, or is undergoing solid organ transplantation. In some embodiments, the HSCT comprises allogeneic HSCT. In some embodiments, the administering of the thawed apoptotic cell population is carried out up to 24 hours prior to said transplantation, at the same time as the transplantation, or is administered until 15 days following said transplantation.

In some embodiments, the subject in need is suffering from a hematopoietic malignancy.

In some embodiments, hematopoietic malignancy comprises MDS, acute lymphoblastic leukemia (ALL), acute myeloid leukemia (AML) or chronic myelogenous leukemia (CML).

In other embodiments, the subject in need is suffering from acute respiratory distress syndrome (ARDS), which, in some embodiments, comprises pulmonary ARDS, and in other embodiments, comprises extrapulmonary ARDS. In other embodiments, the subject in need is suffering from respiratory distress syndrome (RDS), adult respiratory distress syndrome, or shock lung, which, in some embodiments, are alternate names for ARDS. In some embodiments, symptoms of ARDS comprise shortness of breath (dyspnea), rapid breathing (tachypnea), bluish skin coloration (cyanosis), or a combination thereof. In some embodiments, ARDS involves diffuse injury to cells which form the barrier of the microscopic air sacs of the lungs, surfactant dysfunction, activation of the immune system, dysfunction of the body's regulation of blood clotting, or a combination thereof.

In other embodiments, the subject in need is suffering from multisystem inflammatory syndrome in children (MIS-C). In other embodiments, the subject in need is suffering from osteoarthritis.

### Administration

In some embodiments of a method of treating a subject in need comprising administering a thawed frozen formulation of apoptotic cells, wherein a dose of about 140×10⁶-210×10⁶ apoptotic cells is administered. In some embodiments, a dose of about 10-100×10⁶ apoptotic cells is administered. In some embodiments, a dose of about 20×10⁶ apoptotic cells is administered. In some embodiments, a dose of about 30×10⁶ apoptotic cells is administered. In some embodiments, a dose of about 40×10⁶ apoptotic cells is administered. In some embodiments, a dose of about 50×10⁶ apoptotic cells is administered. In some embodiments, 60×10⁶ apoptotic cells are administered. In some embodiments, a dose of about 60×10⁶ apoptotic cells is administered. In some embodiments, a dose of about 70×10⁶ apoptotic cells is administered. In some embodiments, a dose of about 80×10⁶ apoptotic cells is administered. In some embodiments, a dose of about 90×10⁶ apoptotic cells is administered. In some embodiments, a dose of about 1-15×10⁷ apoptotic cells is administered. In some embodiments, a dose of about 10×10⁷ apoptotic cells is administered. In some embodiments, a dose of about 15×10⁷ apoptotic cells is administered.

In some embodiments, a dose of 10×10⁶ apoptotic cells is administered. In other embodiments, a dose of 10×10⁷ apoptotic cells is administered. In other embodiments, a dose of 10×10⁸ apoptotic cells is administered. In other embodiments, a dose of 10×10⁹ apoptotic cells is administered. In other embodiments, a dose of 10×10¹⁰ apoptotic cells is administered. In other embodiments, a dose of 10×10¹¹ apoptotic cells is administered. In other embodiments, a dose of 10×10¹² apoptotic cells is administered. In other embodiments, a dose of 10×10⁵ apoptotic cells is administered. In other embodiments, a dose of 10×10⁴ apoptotic cells is administered. In other embodiments, a dose of 10×10³ apoptotic cells is administered. In other embodiments, a dose of 10×10² apoptotic cells is administered.

In some embodiments, a high dose of apoptotic cells is administered. In some embodiments, a dose of 35×10⁶ apoptotic cells is administered. In other embodiments, a dose of 210×10⁶ apoptotic cells is administered. In other embodiments, a dose of 70×10⁶ apoptotic cells is administered. In other embodiments, a dose of 140×10⁶ apoptotic cells is administered. In other embodiments, a dose of 35-210×10⁶ apoptotic cells is administered.

In some embodiments, a single dose of apoptotic cells is administered. In some embodiments, multiple doses of apoptotic cells are administered. In some embodiments, 2 doses of apoptotic cells are administered. In some embodiments, 3 doses of apoptotic cells are administered. In some embodiments, 4 doses of apoptotic cells are administered. In some embodiments, 5 doses of apoptotic cells are administered. In some embodiments, 6 doses of apoptotic cells are administered. In some embodiments, 7 doses of apoptotic cells are administered. In some embodiments, 8 doses of apoptotic cells are administered. In some embodiments, 9 doses of apoptotic cells are administered. In some embodiments, more than 9 doses of apoptotic cells are administered. In some embodiments, multiple doses of apoptotic cells are administered.

In some embodiments, the apoptotic cells may be administered by any method known in the art including, but not limited to, intravenous, subcutaneous, intranodal, intrathecal, intrapleural, intraperitoneal, parenteral, intra-articular, intramuscular, and directly to the thymus.

In some embodiments, thawed apoptotic cells may be administered therapeutically, once cytokine release syndrome has occurred. In some embodiments, thawed apoptotic cells may be administered therapeutically, once cytokine release leading up to or attesting to the beginning of cytokine release syndrome is detected. In some embodiments, thawed apoptotic cells may be administered therapeutically, wherein the increased cytokine levels are halted or reduced, or wherein the cytokine release syndrome is reduced for fails to occur, and wherein the subject avoids its sequalae.

In some embodiments, an effective amount of thawed apoptotic mononuclear enriched cells is administered to the subject in need. A skilled artisan would recognize that an “effective amount” (or, “therapeutically effective amount”) may encompass an amount sufficient to affect a beneficial or desired clinical result upon treatment, for example but not limited to treating a symptom of the disease, disorder, or condition. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease or symptoms thereof, for example but not limited to respiratory distress. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the apoptotic cell population administered.

The skilled artisan can readily determine the number of cells and optional additives, vehicles, and/or carrier in compositions and to be administered in methods disclosed herein. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %. In other embodiments, about 0.0001 to about 1 wt %. In still another embodiment, about 0.0001 to about 0.05 wt % or about 0.001 to about 20 wt %. In a further embodiment, about 0.01 to about 10 wt %. In other embodiments, about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. The time for sequential administrations can be ascertained without undue experimentation.

A skilled artisan would appreciate that the term “about”, may encompass a deviance of between 0.0001-5% from the indicated number or range of numbers. Further, it may encompass a deviance of between 1-10%, 1-5%, or 1-2.5% from the indicated number or range of numbers. In addition, it may encompass a deviance of up to 10%, 15%, 20%, or 25% from the indicated number or range of numbers.

A skilled artisan would appreciate that the singular form “a”, “an” and “the” include plural references unless the context clearly dictates otherwise. For example, the term “an agent” or “at least an agent” may include a plurality of agents, including mixtures thereof.

In some embodiments, “treating” comprises therapeutic treatment and “preventing” comprises prophylactic or preventative measures, wherein the object is to prevent or lessen the targeted pathologic condition or disorder as described hereinabove. Thus, in some embodiments, treating may include directly affecting or curing, suppressing, inhibiting, preventing, reducing the severity of, delaying the onset of, reducing symptoms associated with the disease, disorder, or condition. Thus, in some embodiments, “treating.” “ameliorating.” and “alleviating” refer inter alia to delaying progression, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A skilled artisan would appreciate that the term “treatment” may encompass clinical intervention in an attempt to alter the disease course of the individual treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder. In some embodiments, improved prognosis comprises reduced hospital stay.

A skilled artisan would appreciate that the term “subject” may encompass a vertebrate, in some embodiments, to a mammal, and in some embodiments, to a human. In some embodiments, the subject in need comprises an adult. In some embodiments, a subject in need comprises a child.

A skilled artisan would appreciate that the term “effective amount” may encompass an amount sufficient to have a therapeutic effect. In some embodiments, an “effective amount” is an amount sufficient to arrest, ameliorate, or inhibit the continued proliferation, growth, or metastasis (e.g., invasion, or migration) of a neoplasia.

Throughout this application, various embodiments disclosed herein may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals there between.

### EXAMPLES

### Example 1: Development of Optimal Cryopreservation Formulation for Freezing and Storage of Apoptotic Cells

Objective: To develop a cryopreservation formulation for freezing and storage of apoptotic cells, wherein upon thawing a high percentage of cells maintain their apoptotic characteristics.

### Methods:

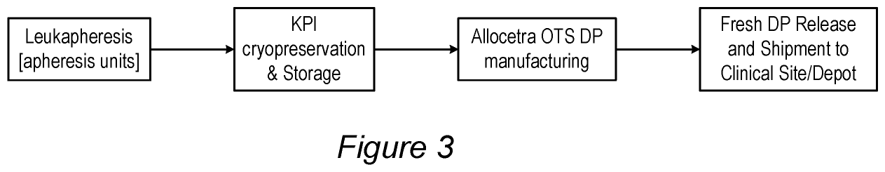

The current manufacturing process of apoptotic cells, for example Allocetra-OTS, consists of 2 main stages as presented in FIG. 3: (1) Collection of apheresates (whole leukapheresis products) and cryopreservation of the Key Process Intermediate (KPI); and (2) Thawing of the KPI and manufacturing of the fresh drug product (DP; i.e., at least 35% of the cell population comprises early and mature apoptotic cells as evaluated by AnV and PI staining, wherein fresh drug product (DP) shows at least 35% AnV+PI+^{DIM} staining and less than 30% AnV+ and PI+^{High} staining). The KPI comprises the whole cell population (consist of ˜50% mononuclear cells and 50% RBC and platelets) received from the apheresis following several steps of wash and centrifugation.

The entire manufacturing process from the starting material up to the DP was performed under Good Manufacturing Practice (GMP) conditions.

Currently, the standard apoptotic cell population known as Allocetra-OTS is formulated as a liquid drug product (fresh) into Hartman's (Ringer's Lactate) solution ready for infusion into the patient in a semi-automated process. (See for example, International Publication No. WO 2016/170541 at Example 1, which is incorporated herein in its entirety.) As the clinical development and use of apoptotic cell populations progresses, and needs for functional DP comprising apoptotic cell populations expands to regions distant from the source of DP manufacturing, making development of a product with a longer shelf-life is crucial. At thaw, the apoptotic population may be evaluated by AnV and PI staining, wherein thawed drug product (DP) shows at least 35% AnV+PI+^{DIM} staining.

An initial study tested two frozen small-scale products, in which the frozen formulation of apoptotic cells was stored for 10 months in liquid nitrogen storage (−196° C.).

To reduce possible known adverse effects caused by the high levels DMSO transfusion, the experiments focused on investigating apoptotic cell (e.g., Allocetra) cryopreservation with DMSO concentrations lower than 10%.

### Results:

To facilitate production of a cryopreserved formulation, manufacturing changes were introduced for the frozen formulation following the cell suspension incubation step, as shown in FIG. 2. These changes were made starting at the step of volume reduction. A first difference was that Fresh Formulations were resuspended in Ringer's lactate solution (Hartman's solution) at this stage, while Frozen Formulations were suspended in Plasma-Lyte (Baxter Healthcare Ltd. Norfolk UK). No changes were made in the steps leading up to the volume reduction media exchange junction.

### DMSO Concentration and Comparison Between Commercial Media and Home Brew Cryopreservation Media

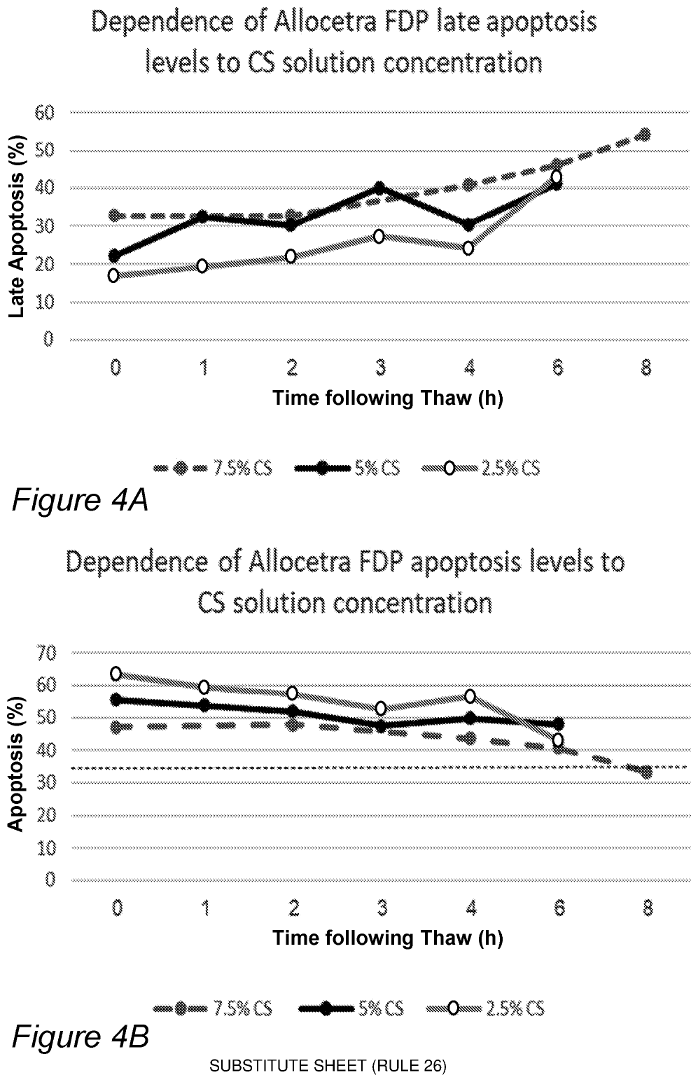

As a component of freezing medium for cell products, DMSO is most often used at a final concentration of 5% to 15%. Preliminary studies were performed to examine the cryopreservation effect with 7.5% DMSO, wherein the results indicated significant damages in Allocetra DP key attributes following thawing. Damage observed included a high level of late apoptotic cells based on key attributes analyzed (% AnV+ and PI+ cells) included an increase in late apoptotic cells population and decrease in apoptotic cells population (FIGS. 4A and 4B).

In addition, four different cryopreservation solutions were tested: 2 commercial solutions and 2 home-brew formulations. The CryoStor CS5 and CS10 commercial solutions (BioLife Solutions) have been diluted 1 by 1 with the Allocetra cell suspension, creating CS-2.5 and CS-5 media, respectively (2.5% and 5.0% DMSO, respectively).

The Allocetra cell suspension was prepared as briefly provided herein below.

Peripheral mononuclear blood cells were collected using a leukapheresis procedure from a single donor. The cells were cryopreserved as in the liquid product and undergone similar processing to the liquid product up to the LOVO media reduction and formulation. The LOVO formulation and resuspension for the frozen formulation was made into a PL148 solution, instead of the Ringer's lactate solution.

The LOVO resuspension volume is also reduced to 220 mL resulting in higher cell concentration (ranging from 50-80 million of cell to ml). The cells were then irradiated at 4000 cGy and brought to the required cell concentration, which will allow a 10 billion cell in a fixed dose product of 400 ml following the 1:1 dilution with the cryopreservation solution.

Allocetra populations that contain a cell concentration greater than 60 million cells/ml following LOVO would be diluted with PL148 before they are mixed with the cryoprotectant agent.

Following the mixing with the cryoprotectant, the cells were gradually frozen and transferred to liquid nitrogen, as described herein, for long-term storage.

The two home-brew formulations tested were based on common cryopreservation media, consisting of 5% or 2.5% DMSO (v/v) with 3% (w/w) human serum albumin (HSA) in PlasmaLyte-148 (PL148, Baxter Healthcare, Ltd) solution. Cells were thawed by placing bags containing frozen formulations of cells in a pre-heated 37° C. water bath and gently agitating until no ice particles remain. (less than 5 minutes). Thawed cells were then kept at the ambient temperature (Rm. Temperature) throughout the 3 hours of the study duration.

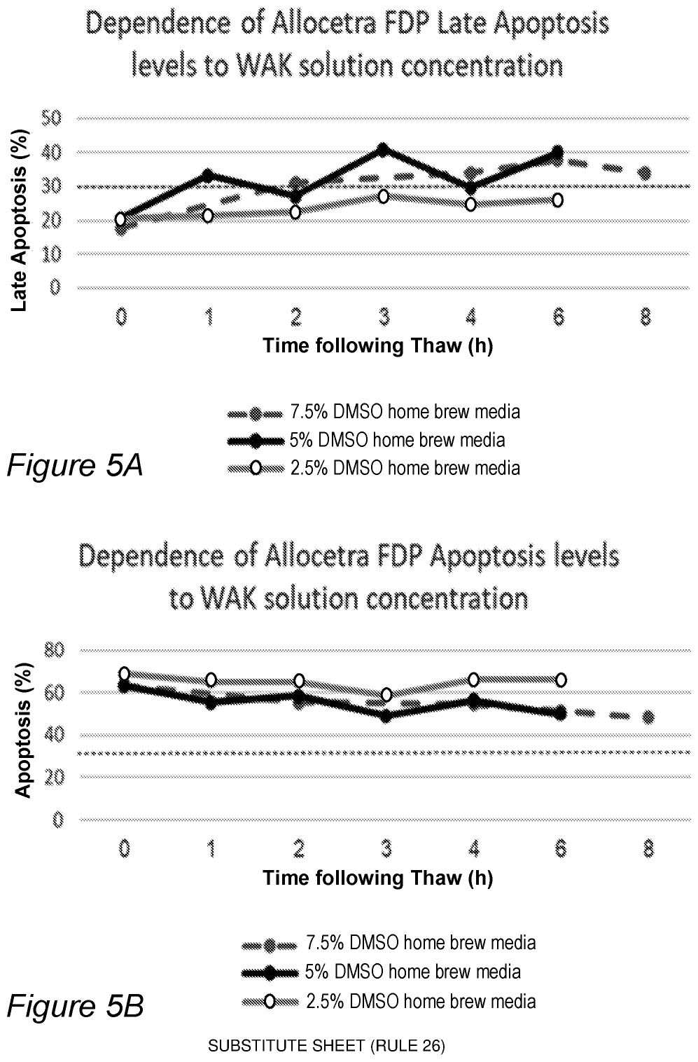

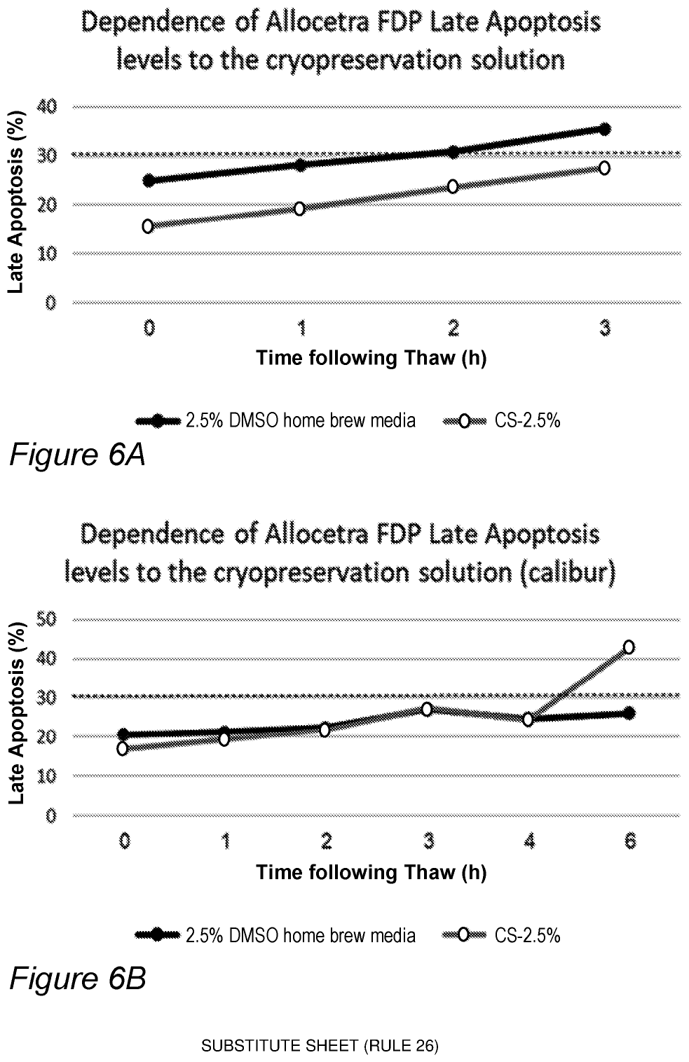

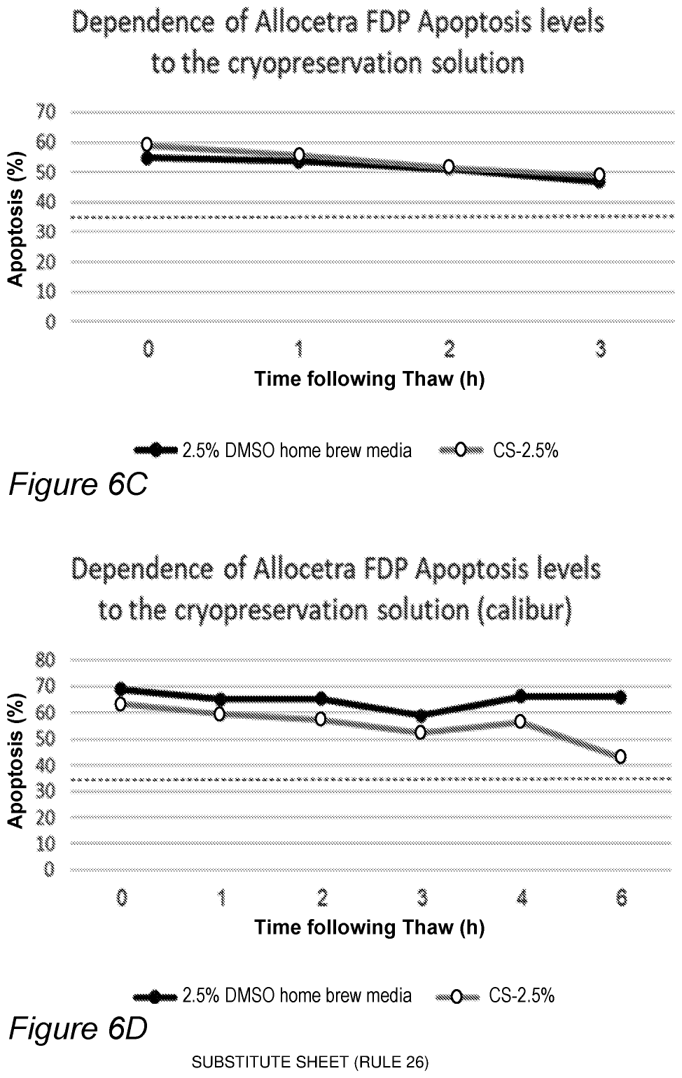

The effects of these formulations were tested in view of the Allocetra DP key attributes in several studies immediately following thawing and on an hourly basis until 3-8 hours after thawing. Both the 2.5% DMSO commercial and the home-brew formulations presented low late apoptosis levels (no more than 30% of the cell product is AnV+PI^{HI}) and good recovery results (FIGS. 4A and 4B for commercial formulations, and FIGS. 5A-5B for home-brew, and FIGS. 6A-6D for comparing commercial and home-brew formulations.). However, the commercial CS-2.5 media (CS5 diluted with PL148) consistently offered slightly better protective effects to the post-thaw Allocetra cells in terms of lower percent late apoptotic cells in the population as a whole level (FIGS. 4A-4B, FIGS. 5A-5B, and FIGS. 6A-6D), hence CS-2.5 was chosen as the basis for the lead formulation.

The Cryostor® cell cryopreservation medium CS5 (Biolife Solutions) was selected as the freezing medium. Cryostor® was purchased as a sterile product and contains DMSO (5%) and additional proteins/molecules, which support the cells during the freeze-thaw cycles. In the final formulation, the Cryostor was diluted 1:1 with the PlasmaLyte cells suspension and the DMSO was diluted to 2.5%. The final frozen apoptotic formulation, prepared using CS5 diluted 1:1 to final 2.5% DMSO with Plasmalyte, comprised at least 35% apoptotic cells (early and mature), less than 30% late apoptotic cells,

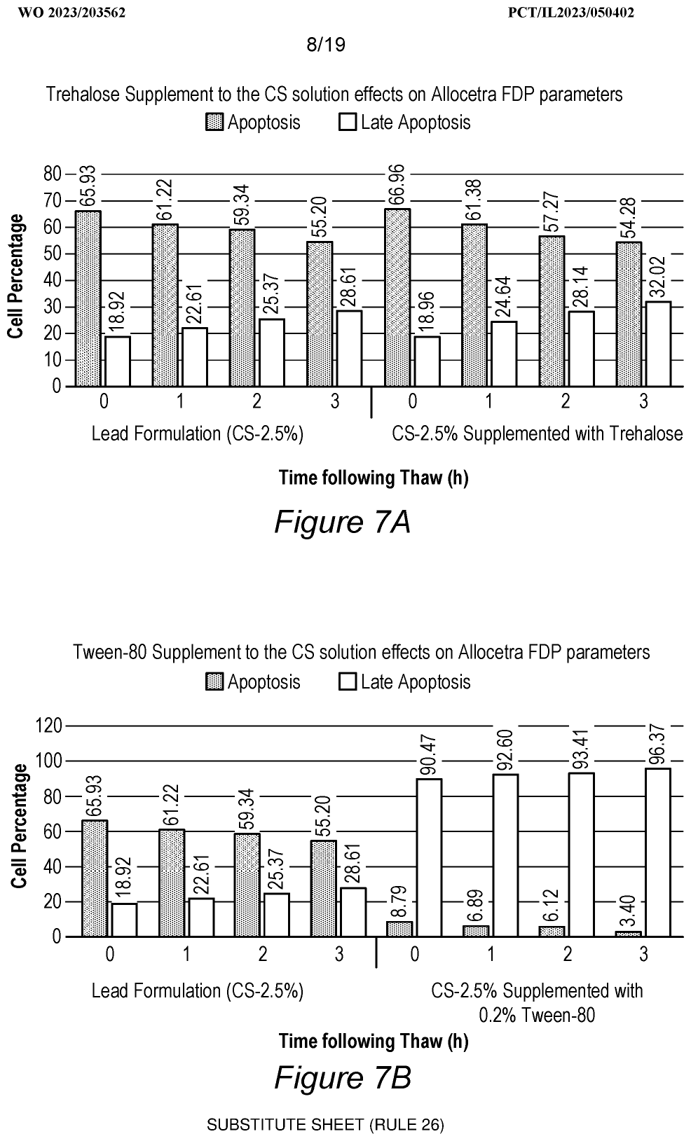

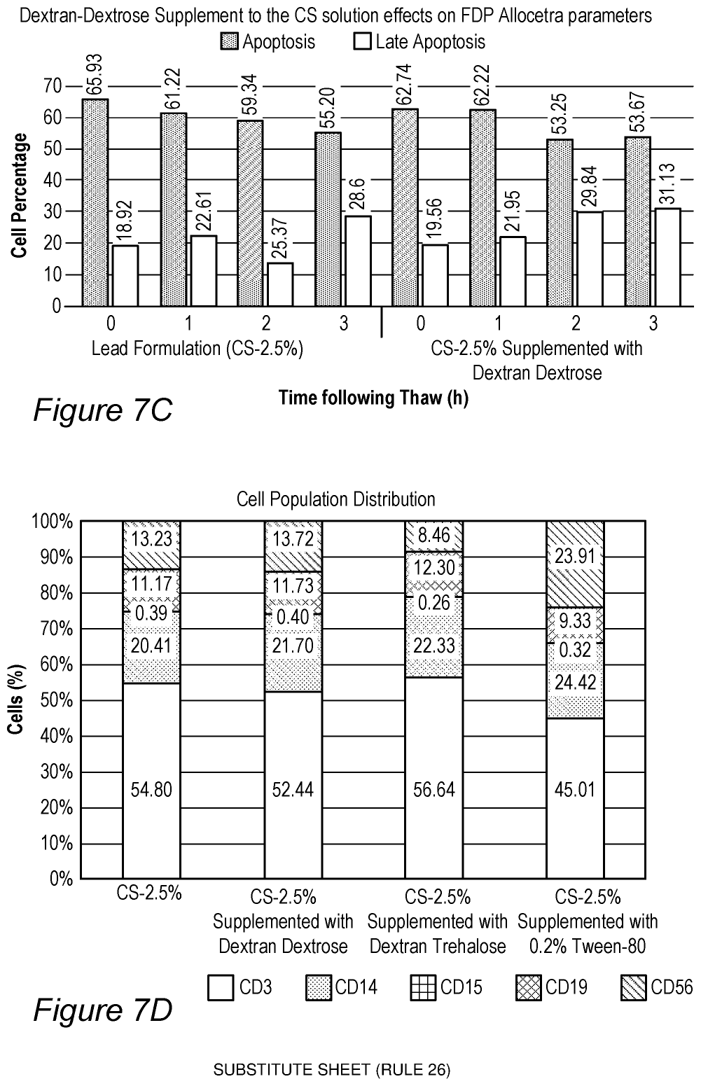

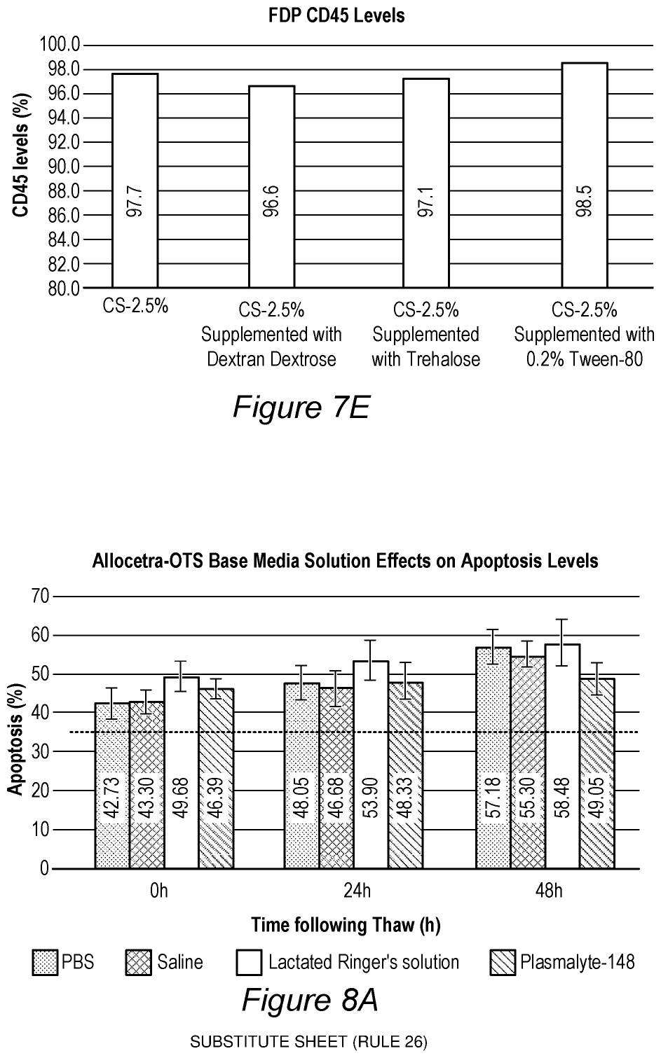

The effectiveness of this medium was also tested in the presence of a variety of excipients (Trehalose, Tween-80 and the Dextran-dextrose solution) that purport to act as additional stabilizers for the cells or to provide physiological osmolality. But no significant differences were observed in the percent of late apoptotic cell levels, apoptosis (early and mature) levels, or cell population markers (FIGS. 7A-7E) were observed between the excipients supplemented media and the control cryopreservation medium, therefore the lead cryopreservation formulation recipe was retained without additives.

Interestingly, the selected freezing medium optimized herein, contained a lower DMSO concentration (2.5%) than generally used in the field, which surprisingly led to the most protective effect on apoptotic cells (e.g., Allocetra cells) compared to the commercial solution with the higher DMSO concentrations (7.5% or 5%). (FIGS. 4A and 4B)

Formulation into Cryopreservation Base Media Solution

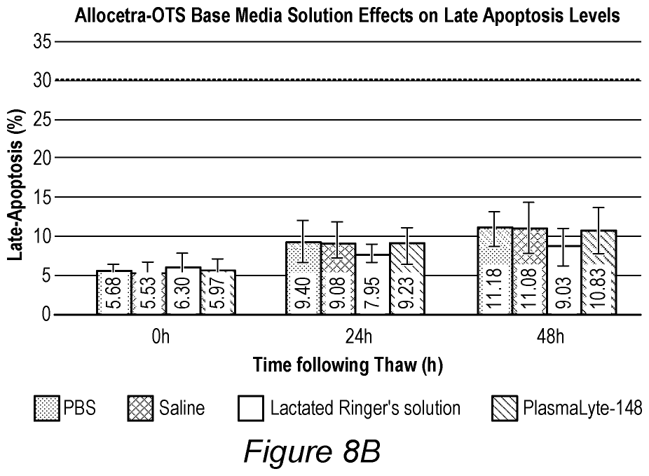

The base media used to prepare the cryoprotectant agent (CPA) can also affect the cryopreservation outcome. Typically, an isotonic electrolyte solution approved for infusion may be used as the solvent for the cryopreservation solution preparation. The PL148 solution is a common cryopreservation base solution. The analysis using PL148 presented comparable apoptosis and late apoptosis results to those of the PBS solution but slightly higher than the Lactated Ringer's solution. FIGS. 8A and 8B

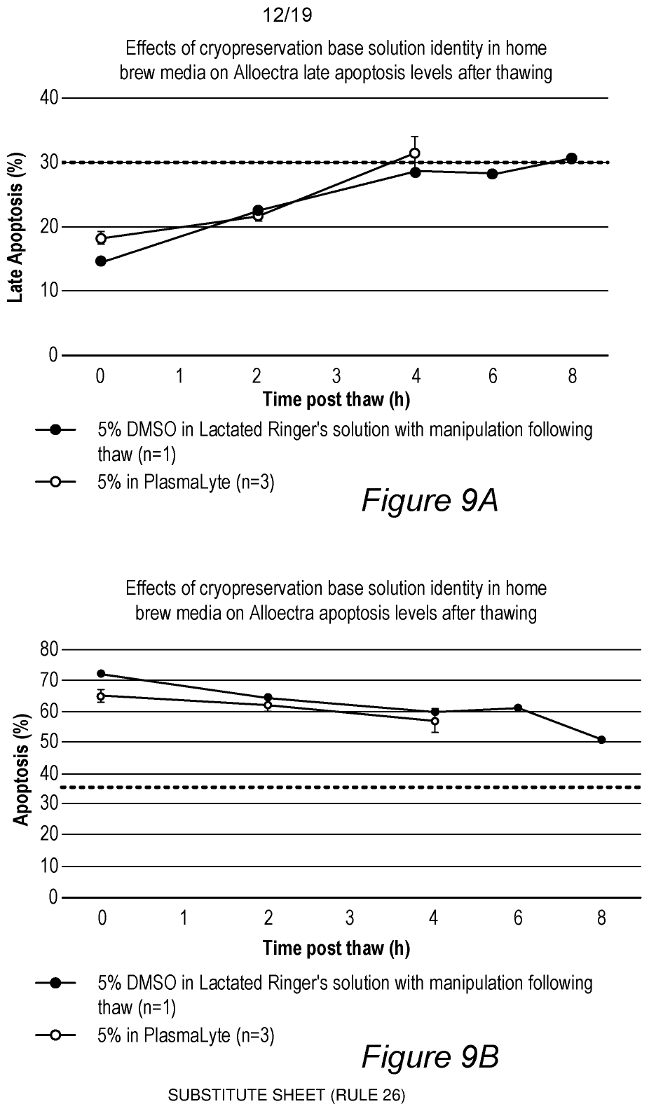

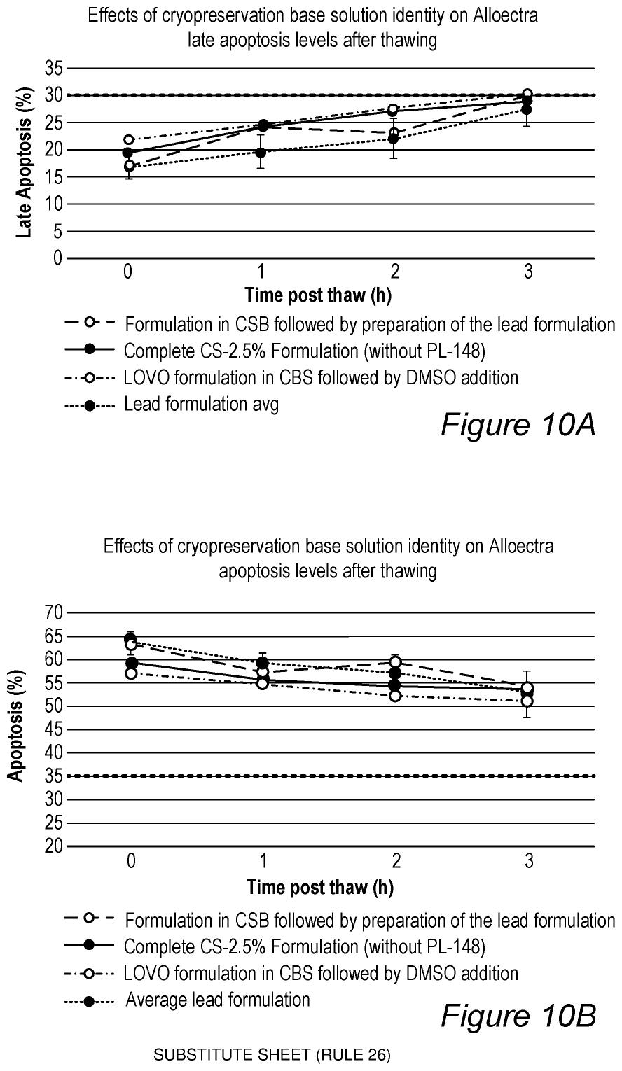

The frozen formulation base solution (formulation minus the apoptotic cells) identity was determined at an early stage of the formulation development. Initially the CPA solution (WAK's Cryo-Sure DMSO) was introduced to a Allocetra cell suspension solution after the LOVO processing (consisting of the Lactated Ringer's solution). The Plasmalyte-148 solution was tested soon after, as a possible alternative since it serves as a conventional solvent for cryopreservation and is currently used in the KPI freezing process. The different cryopreservation formulations were tested with a 5% DMSO home-brew solution with similar results. Once the percentage of DMSO for frozen product had been selected, the Cryostor® Basal medium (CSB) solution was tested as an alternative for the cryopreservation base media solution. It was presumed that the Allocetra cells would benefit from a complete CryoStor medium since it is uniquely formulated to address the molecular-biological aspects of cells during the cryopreservation process, in aim to improving in post-thaw cell viability and function. The apoptotic cells (Allocetra) were formulated into the CBS solution using the LOVO method. The CPA solution containing the DMSO component was introduced to the cells in the CSB suspension immediately prior to the gradual freezing by either mixing the cell suspension 1:1 with the CS5 solution, by mixing the cell suspension 1:1 with Plasmalyte-148 and CS-10 mixture or by introducing WAK DMSO directedly to the cell suspension to a final concentration of 2.5%. The formulations that were created by diluting the cell suspension with CS solutions had produced good results, but the lead formulation in which the cells are formulated directly into Plasmalyte-148 using the LOVO had provided slightly better outcome. (FIGS. 9A-9B and 10A-10B).

### Formulation and Introduction of a Freezing Medium Base Solution

The end-of-manufacturing (LOVO volume reduction and product formulation) and the cryopreservation procedures overlap one with another, hence the pre-freeze processing and the Allocetra reformulation can be incorporated into a single step in the process flow as a part of process refining strategy by creating a seamless transition between steps. Hence, the volume reduction and formulation were performed directedly into the cryopreservation base media (PL148 solution). The LOVO retentate volume was set to 220 mL creating a cell suspension of 40-75×10⁶ cells/mL, enabling the creation of a frozen product with a fixed dose of 10 billion cells (with a 20% margin for error).

### Cell Concentration

The cell concentration per unit volume of cryoprotectant medium can affect cell survival. Conventional concentrations employed for the cryopreservation of hematopoietic stem cells in cell therapy range from 20 to 80×10⁶ cells/mL. For the purposes of creating a frozen formulation of apoptotic cells that is at least 35% apoptotic cells (early and mature apoptotic cells), a fixed dose frozen apoptotic cell (Allocetra) product (FDP) was developed.

The fresh Allocetra (apoptotic cell) formulation administration was initially based on body size, from the perspective that this reduces inter-subject variability in drug exposure. However, because of the advantages of fixed dosing (increased convenience, elimination of wastage, improved safety resulting from a reduced chance for dosing errors, and improved compliance), the weight-based dosing concept was re-evaluated and abandoned in favor of the fixed-dose regimen.

The decision to produce a fixed dose frozen apoptotic cell product was guided by the same considerations. The dose of the frozen apoptotic cell product was chosen based on the clinically tested fresh product fixed dose, 10 billion cells per batch. which will allow administration of either a 5-billion or the full 10-billion fixed dose products to a subject in need. The FDP manufactured batch will be cryopreserved in 4 bags of 100 ml each (the total cryopreserved volume will be 400 ml). The frozen product estimated concentration will be about 25×10⁶ cells/ml±20%.

The highest and lowest concentrations of frozen Allocetra are 30×10⁶ and 20×10⁶ cells/ml, respectively, both derived from the fixed dose chosen with a margin of error of 20%.

### Cryopreservation Vessel

The Origen CryoStore™ cryobags (OriGen Biomedical, Inc., USA), which are specifically designed for cryopreservation of injectable solutions and storage in liquid nitrogen, were selected as the cryopreservation vessel for the apoptotic cell population. Following product filling (four bags, each filled with 100 mls frozen formulation for a total of 400 mls and a total of about 10 billion±20% apoptotic cells), the cryobags were placed in aluminum cassettes to protect the product from mechanical damage that might compromise the structure of the container and its sterility, but also ensures a consistent, homogeneous thermal profile across the length, and thickness of cryobags during freezing, which is essential to ensure homogeneous cell recovery between preparations.

Process development studies are still ongoing to determine the optimal fill volume and stability of the frozen product.

### Long Term Storage

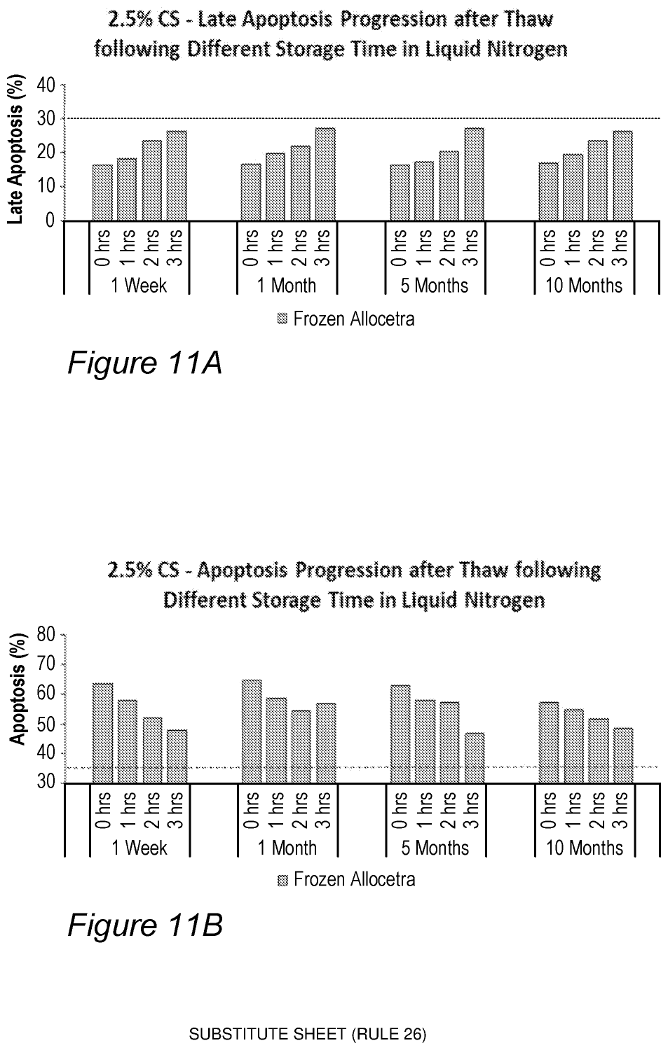

The results of storing the frozen formulation of apoptotic cells for at least 10 months in liquid nitrogen are presented in FIGS. 11A and 11B. FIG. 11A shows that there was minimal percent (%) change in the number of late apoptotic cells in the population as a whole, following liquid nitrogen storage for 1 week, 1 month, 5 months, and 10 months. Significantly, FIG. 11B shows that the % apoptotic cells (early and mature apoptotic cells) in the population as a whole, was not reduced following liquid nitrogen storage for 1 week. 1 month, 5 months, and 10 months.

Storage in liquid nitrogen of fixed dosed product is expected to show similar stability in the % apoptotic and late apoptotic cells following thawing of frozen formulations stored for 10 months or more.

### Product's Quality and Biological Attributes

The current process for freezing an apoptotic mononuclear enriched cell population is as follows. Briefly, following induction of apoptosis the cells were irradiated at 4000 cGy and brought to the required cell concentration, which allows for 10 billion cells in the FDP having a final volume of 400 ml and following the 1:1 dilution with the cryopreservation solution. Fixed dose products that contain a cell concentration greater than 60 million cells/ml following LOVO were diluted with PL148 before being mixed with the cryoprotectant agent. Following the mixing with the cryoprotectant, the cells were gradually frozen and transferred to liquid nitrogen for long-term storage. What follows are the steps which were taken to access the quality and biological attributes of the frozen formulation of apoptotic cells, after quick thawing, compared with freshly prepared apoptotic cells.

Following preparation of an apoptotic mononuclear enriched cell population for freezing, and the selection of the freezing media formulation, several studies were performed, to assess the product's quality and biological attributes in the new formulation. Tests were performed on the frozen apoptotic cell product immediately after thawing (T=0) and every hour up to 3 hours, to simulate in-use conditions (estimated time from thawing to end of administration is approximately 1.5-2 hours for 60-100 ml bags).

Basic Quality Test Results: The target population of the apoptotic cell product is identified as mononuclear cells (MNCs) and was evaluated by staining for CD45 (a common marker for all the MNCs subpopulations). The main population in Allocetra-OTS is the MNCs population (CD45 positive cells). The CD45 levels should be over 85% for the population of Allocetra to be considered CD45 positive. The product is typically characterized by a very high proportion of MNCs, above 85%. And the MNCs may be characterized also by the profile of their subpopulation composition.

The results presented in Tables 1 and 2 below show that measure of % apoptosis (apoptotic (early & mature) or late) and measure of CD45+ for the frozen and fresh preparations, remains within a similar range. CD45+ is a marker for mononuclear cells (MNC), which comprises a technical characteristic of the apoptotic cells being frozen.

The characterization of MNCs was based on the membranal expression of human CD45, which is a common marker of all hematopoietic cells (blood cells), except for mature erythrocytes (red blood cells) and platelets. Drug product cells (Fresh and Frozen/thawed apoptotic cell batch) were stained using a triple mix of fluorescence antibodies: Anti-human CD45 as an MNCs marker, Anti-human CD42a, and Anti-human CD235a, as a platelets and erythrocytes marker, respectively.

TABLE 1
Annexin V/Propidium Iodide (AnV/PI) flow cytometry Analysis
for Apoptotic (early & mature) and Late Apoptotic cells
in fresh and frozen formulation products

| | Apoptosis (%) (AnV + PI− or PI^{DIM}) | | | | | |
|---|---|---|---|---|---|---|
| | Frozen Formulation | | | | Fresh Formulation | |
| | T = 0* | T = 1 h | T = 2 h | T = 3 h | T = 0 | T = 96 h |
| AVG | 60.3 | 56.2 | 53.4 | 49.8 | 47.63 | 59.66 |
| SD | 2.8 | 1.3 | 3.0 | 2.3 | 4.63 | 6.02 |
| | Late Apoptosis (%)(AnV + PI^{HI}) | | | | | |
| | Frozen Formulation | | | | Fresh Formulation | |
| | T = 0* | T = 1h | T = 2h | T = 3h | T = 0 | T = 96 |
| AVG | 15.5 | 19.2 | 22.2 | 27.3 | 6.62 | 7.66 |
| SD | 2.7 | 3.5 | 3.6 | 3.3 | 2.01 | 2.43 |
| N | 3 | 3 | 3 | 3 | 30 | 13 |

*Upon thawing

Percent apoptosis within the population reflects the presence of early apoptotic cells (AnV+PI−) and mature apoptotic cells (AnV+PI dim). Mature apoptotic cells are characterized as AnV positive and PI dim (low level of PI staining) in contrast to the late apoptotic cells—AnV positive and High PI, or to the early apoptotic cells, which are characterized by AnV positive cells and negative staining to PI.

TABLE 2
Flow cytometry Analysis for Purity (CD45 positive cells
population) in fresh and frozen formulation products

| CD45 positive cells (%) | |
|---|---|
| Frozen Formulation | Fresh Formulation |

| | T = 0* | T = 3 h | T = 0 | T = 96 h |
|---|---|---|---|---|
| AVG | 96.3 | 95.4 | 94.2 | 94.62 |
| SD | 1.7 | 1.4 | 2.8 | 3.7 |
| N | 5 | 17 | 8 | 12 |

*Upon thawing

The content of apoptotic cells is an important quality attribute of the product. To determine apoptotic state, Annexin V (AnV) and propidium iodide (PI) staining were performed. During the initial stage of apoptosis, membrane bound PtdSer translocates from the inner, cytoplasmic layer leaflet of the cell membrane to the outer, extracellular layer leaflet of the cell membrane, thus exposing PtdSer to its immediate environment and making it accessible to AnV binding. Following PtdSer exposure, the apoptotic process progresses and continues with activations of additional caspases. Two sub-populations of apoptotic cells may be derived from this dynamic process: apoptotic cells characterized by single stain to AnV no PI staining, and mature apoptotic cells characterized by AnV and low PI staining (dimly PI fluorescent population due to the progression of the membrane permeabilization process which leads to gradual loss of membrane integrity). Apoptotic cells are eventually engulfed by phagocytes, if cells are not phagocytosed, the plasma membrane gradually loses its integrity. Loss of plasma membrane integrity can be detected by nonselective and massive uptake of fluorescent dyes such as PI, which leads to high intensity of PI staining on the cells. The PI enters the nucleus and intercalates within the DNA double strand and may serve as a biomarker for late apoptotic or necrotic cells.

The distinction between the two apoptotic sub-populations (early and mature), was achieved following optimization of the analytical method and improvement in the flow cytometry sensitivity. The optimized method, with a more accurate gate strategy allows a more accurate measurement of the apoptotic population. This led to an update in release specifications used throughout, wherein apoptotic cells encompass at least 35% early (AnV+PI−) and mature (AnV+PI^{Dim}) apoptotic cells and less than 30% late apoptotic cells (AnV+ and PI+^{High}).

Determination of the percentage of apoptotic cells population in Drug Product samples was performed using MEBCYTO apoptosis kit (MBL International Corporation, Woburn, MA), includes AnV-FITC conjugated and PI staining. Thus, apoptotic cells appeared as positive to AnV staining, with negative or low PI staining. The late apoptotic cells were all brightly stained by PI and appeared as a peak at very high fluorescence values (high intensity of PI staining) due to the significantly absorbance of the dye.

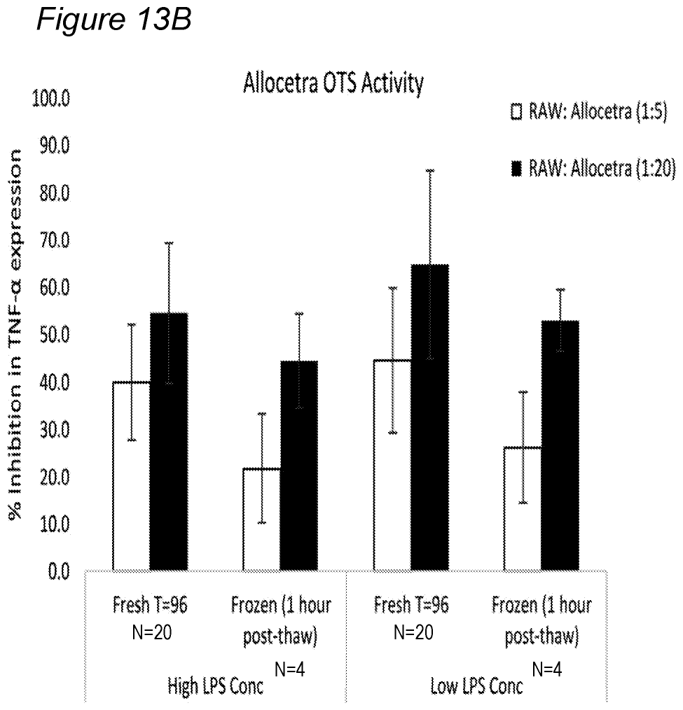

As seen in Tables 1 and 2 and FIGS. 13A and 13B, results of most basic quality parameters of the frozen formulations remain within the range of the fresh formulation. The only parameter which seems to be affected in thawed frozen formulation apoptotic cells was the increased percentage of late apoptotic cells. Following the process of thawing the frozen formulation of Allocetra-OTS cells, an increase in the late apoptotic cell abundancy was observed. A possible explanation for this phenomenon may be the rapid progress of apoptotic cells to progress to a terminal apoptosis phase due to the freezing and or thawing process.

The late-stage apoptotic cell population, which were acquired from progression of the apoptosis process are termed “secondary necrotic” cells in the literature. These late-stage apoptotic cells are characterized by cytoplasmic swelling and plasma membrane damage. No safety concerns are expected from the higher rate of secondary necrotic population, since the cells are also taken up by phagocytosis (in contrast to the primary necrotic cells). Based on the evidence from the relevant literature (Sachet, M., Liang, Y. Y., & Ochler, R. (2017). The immune response to secondary necrotic cells. Apoptosis: an international journal on programmed cell death, 22 (10), 1189-1204; Silva et al., (2010) FEBS Letters Vol. 584, Issue 22, Pages 4491-4499), the late apoptotic population also dampens pro-inflammatory signals and may lead to different immune responses compared to the classical primary necrosis cells.

Biological Activity-Potency Assay: The biological activity of Allocetra cells was also compared between the two formulations (Fresh and Frozen), using the potency test. The apoptotic product activity is described as the inhibition of TNF-α expression from activate murine macrophages. The activity of the frozen product was tested 1-hour post-thaw and was compared to the fresh product activity at the beginning and end of the shelf life (T=0, FIG. 13A; T=96, FIG. 13B).

The purpose of the analysis of percent (%) inhibition of TNF-alpha expression is to determine by flow cytometry analysis the potential of Drug Product apoptotic cells to induce immune modulation by reduction of the TNF-α expression in macrophages in the presence of two Lipopolysaccharide (LPS) concentrations (high LPS-0.05 ng/ml and low LPS-0.025 ng/mL). This assay was based on evaluation by intracellular staining and flow cytometric analysis. Murine macrophages cell line (RAW 264.7 cells) interaction with Allocetra cells led to a reduction in the LPS-induced up-regulation TNF-α. The reduction in the TNF-α positive cells was calculated and describes the percent inhibition mediated by the Allocetra-OTS at two different rations (1:5 and 1:20 of RAW: Allocetra) in a dose-dependent pattern. Essentially, macrophages were activated with LPS to secrete TNF-alpha and then co-incubate with Allocetra, which inhibited this effect.

As shown in FIG. 13B, the average activity of frozen batches (1-hour post-thaw) seems to be slightly lower compared to that of the fresh batches at the end of the shelf life (T=96) as demonstrated by ICS. However, the range of results for the 4 frozen batches seems to be within the range of the 20 “fresh” batches. Given the inherent variability of this cell-based assay, and the inherent variability between batches, the slight differences observed do not seem to be related to the change in formulation.

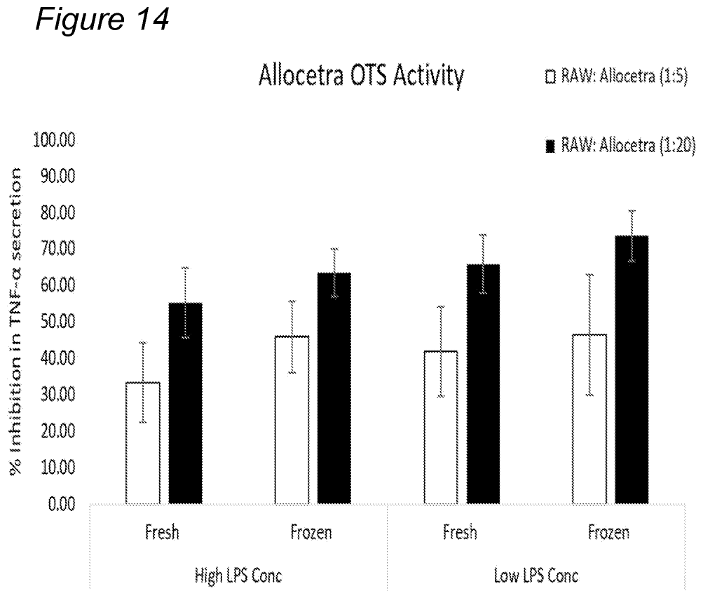

Additional support for the similarity between the frozen and fresh formulations was provided by an ELISA-based potency assay, as shown in FIG. 14. This assay was based on the evaluation of cytokine secretion by mouse TNF-α ELISA. The apoptotic product activity is described as the inhibition of TNF-α secretion from activated murine macrophages. The average activity of frozen batches was compared to the average activity of fresh batches during the shelf life (T=0 to T=96 hours).

The efficacy of the frozen formulation product was also evaluated using the murine CLP model, considered the gold standard animal model of reproducible sepsis. Two different batches were tested by 4 independent studies in order to evaluate the efficacy of the frozen formulation batches in this model. Allocetra-OTS thawed from frozen formulations was administered up to 1.5 hours post thaw in combination with fluid resuscitation and antibiotic treatment (Ertapenem) standard of care (SOC).

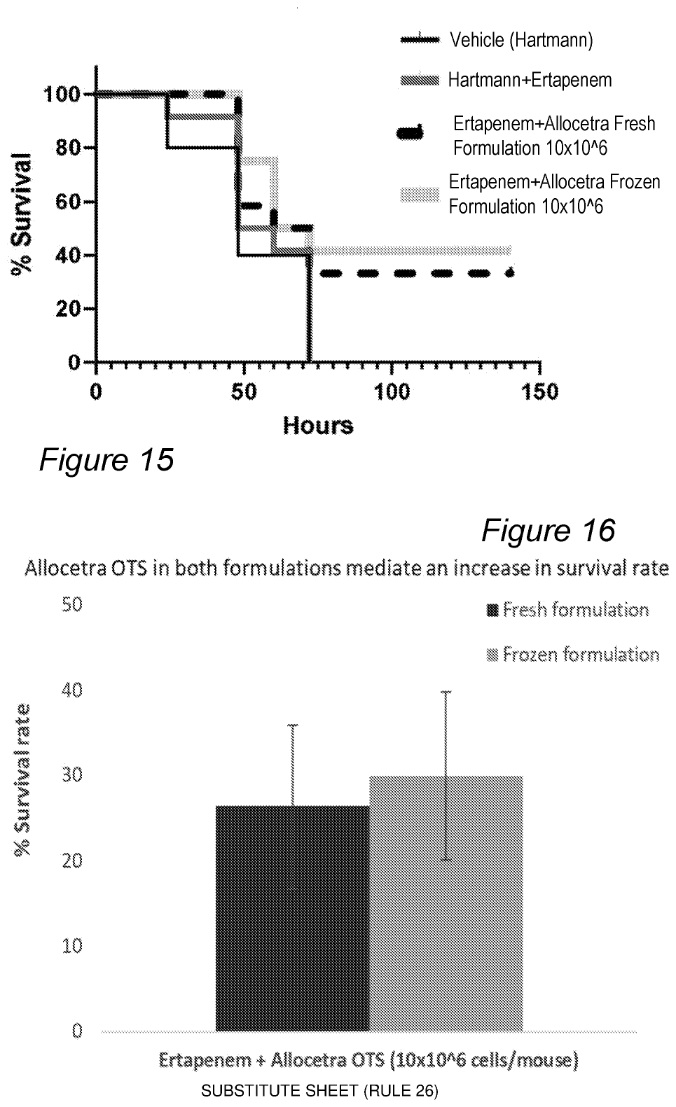

As indicated in FIG. 15 and FIG. 16, a significant effect of the frozen formulation batch was demonstrated by the improvement in the survival rate compared to the standard of care group was observed. A similar effect was observed also by the fresh formulation batch. Considering the variability of the model, the results indicate the similarity between the two formulations in terms of biological activity and in vivo efficacy.

### Summary:

The above results lead an optimized cryopreservation formulation comprising (1) about 10 billion apoptotic cells, (2) an isotonic crystalloid solution comprising 140 mm/L sodium, 5 mmol/L potassium, 1.5 mmol/L magnesium, 98 mmol/L chloride, 27 mmol/L acetate, and 23 mmol/L gluconate, and (3) DMSO at a final concentration of 2.5%, which can be stored at −196° C. for at least 10 months, wherein upon thawing, the formulation maintained the biological activity of the apoptotic cells. Moreover, the results using a thawed frozen apoptotic cell formulation indicate beneficial effect on the survival rate, as observed also for fresh formulation batches (FIGS. 15 and 16). Overall, Allocetra-OTS-treated mice survived approximately 30% longer than antibiotic-treated CLP mice in both formulations. These findings provide additional support for the biological activity and efficacy of Allocetra-OTS thawed from frozen formulations, as described herein.

Conclusion: Following the cryopreservation/thawing process of Allocetra cells in presence of formulation containing DMSO, a reasonable increase in the late apoptotic and mature apoptotic cells (compared to the presence of these populations in the liquid Allocetra product is observed. Cryopreservation is known to cause damage to frozen cells and thus affects cell survival. (Matsushita et al. (2003) Cell Transplantation, Vol. 12, pp. 109-121.) Since the biological activity of the product is derived mainly from the early apoptosis population and not from the mature and late apoptotic population it was expected that the product activity will be also impacted and reduced following the cryopreservation/thawing process. As indicated in FIGS. 13-16, following the thawing of the frozen product, the Allocetra cells mediated almost the same activity as the fresh Allocetra cells, despite the increase in the mature and late apoptotic cells. The Allocetra activity from frozen batches was tested by in vitro models (Intracellular staining and ELISA, FIGS. 13A, 13B, and 14, respectively) and by in vivo model (CLP model, FIGS. 15-16) and compared to the activity derived from the fresh batches. The ability to freeze and thaw apoptotic cell populations as disclosed herein, and maintain the functionality of the cell despite the increase in the mature and late apoptotic population was therefore surprising and unexpected.

### Example 2: Analysis of Late-Stage Apoptotic Cells

Objective: To compare the appearance on the AnV/PI flow cytometry histogram of the late-stage apoptotic cells population with control necrotic cells and determine overlapping gating in flow cytometry.

### Methods:

Naïve PBMCs were received from the leukapheresis process and were induced to necrosis process as follow. Cells were incubated overnight under 5% CO₂ at 37° C. and then transferred to the liquid nitrogen tank for prolonged storage before the AnV/PI staining. Cells were thawed at 37° C. for 2 minutes. The thawed cells were stained with Annexin V (AnV) and Propidium Iodide (PI) to assess the prevalence and state of the necrotic cells. Necrosis induction promoted all of the cells to a necrotic state.

### Results:

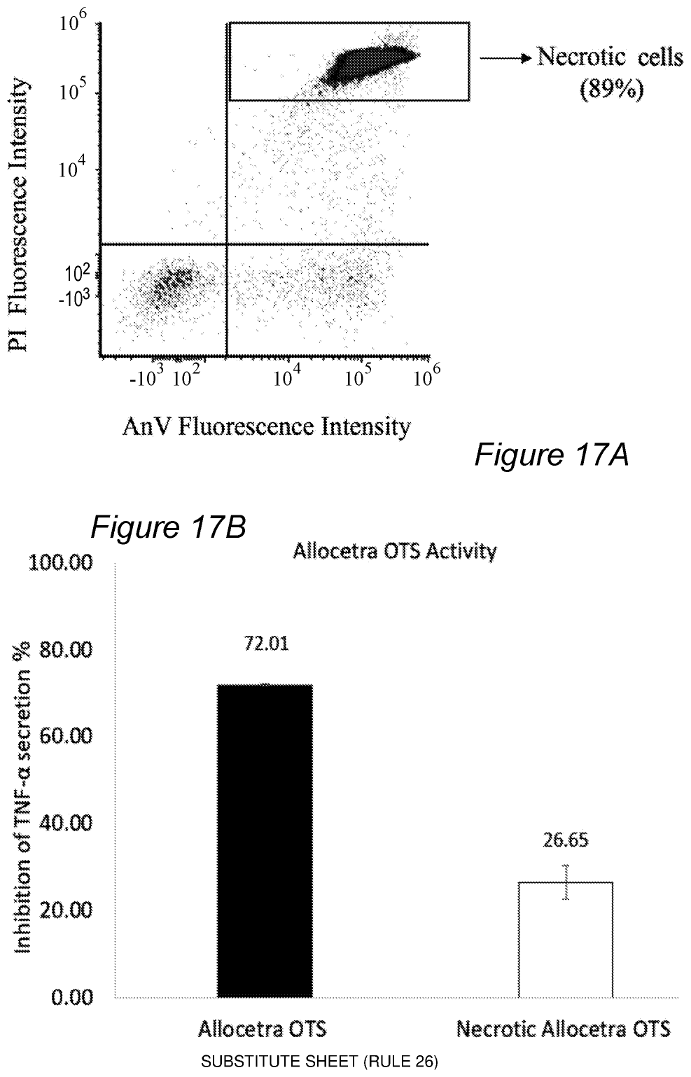

The FIG. 17A shows that following the necrosis induction, more than 85% of the total PBMCs were induced to necrosis phase and appeared in the AnV+, PI+ necrosis control gate.

The necrotic cell control was used as reference cells to characterize the flow cytometry pattern of late apoptotic cells, for example late apoptotic cells as identified in FIG. 1 of Example 1. Analysis showed that the late apoptotic cells overlapped by at least 80% with the generated control necrotic cells (FIG. 17A and data not shown).

FIG. 17A shows that about ˜90% of the total late-stage apoptotic/necrosis cells colocalized with the defined necrosis control gate. Thus, the late-stage apoptotic cells identified as part of the total Allocetra/product population produced (See, Example 1) overlap with flow cytometry sorted necrotic cells. Further, necrotic cells have significantly reduced activity compared with apoptotic cells (FIG. 17B).

### Summary:

Based on the results of the flow cytometry comparison presented here, Allocetra late apoptotic cells are those cells that localized at the same gate as greater than (>) 85% necrotic cells are localized under the same setting of AnV/PI staining and same setting for flow cytometry acquisition and voltages.

### Example 3: Analysis of Apoptotic Induction Time

Objective: To evaluate the apoptosis induction time frame.

Methods: The standard frozen product, as described in Example 1 undergoes a 6 hr incubation with methyl prednisone, as described therein. Here, 1 hr and 6 hr incubations were performed, followed by analysis of the % of apoptotic cells and % of late apoptotic cells. The cell suspension following incubation underwent volume reduction and media exchange with the LOVO cell processing system into PlasmaLyte-148 solution. The cells are then irradiated diluted to the fixed dose and mixed 1:1 with CS-5 solution followed by gradual freezing.

### Results:

Table 3 shows the rates over time of % population of apoptotic and late apoptotic cells following a one-hour incubation rate.

TABLE 3
Percent (%) apoptotic and % late apoptotic cells
Apoptosis Rate early (AnV+PI−) and mature (AnV+PI^{Dim}) apoptotic cells:

| | | Standard (6 hours |
|---|---|---|
| Time post | 1 hour Apoptosis | Apoptosis Induction) |
| induction (hr) | Induction | Frozen Product (N = 5) |
| T = 0 | 60.69 | 58.89 ± 2.82 |
| T = 1 | 60.61 | 55.55 ± 1.46 |
| T = 2 | 55.94 | 51.35 ± 3.52 |
| T = 3 | 51.45 | 48.71 ± 2.43 |

Late Apoptosis Rate

| | | |
|---|---|---|
| T = 0 | 13.6 | 15.42 ± 1.88 |
| T = 1 | 15.31 | 19.28 ± 2.67 |
| T = 2 | 21.22 | 23.62 ± 3.50 |
| T = 3 | 27.63 | 27.54 ± 3.67 |

% Cells Recovery from Thawing Rates: (Cell concentration
before the freezing/Cells concentration following the thawing)

| | | |
|---|---|---|
| T = 0 | 100 | 100 |
| T = 1 | 99.4 | 97.9 ± 2.2 |
| T = 2 | 97.7 | 96.8 ± 2.3 |
| T = 3 | 96.3 | 94.4 ± 1.7 |

% Cells Recovery from Freezing Rates: (Cells concentration,
T = 0 of thawing/Cells concentration T = X of thawing)

| Time post | | Standard (6 hours incubation) |
|---|---|---|
| induction (hr) | 1 hour Incubation | Frozen Product (N = 5) |
| T = 0 | 99.8 | 102.3 ± 2.3 |
| T = 1 | 98.9 | 99.8 ± 3.4 |
| T = 2 | 97.3 | 99.0 ± 3.0 |
| T = 3 | 95.8 | 96.3 ± 2.7 |

The apoptosis induction time range of the frozen product was validated for 1 and 6 hours with similar findings and characteristics (Similar late apoptosis, cell recovery rates, and activity with a similar appearance).

While certain features of the frozen formulations of apoptotic cells and methods of freezing have been illustrated and described herein, many modifications, substitutions, changes, and equivalents will now occur to those of ordinary skill in the art. It is, therefore, to be understood that the appended claims are intended to cover all such modifications and changes as fall within the true spirit of the formulations disclosed herein.

What is claimed is:

1. A frozen formulation comprising:
(a) a population of apoptotic mononuclear enriched cells;
(b) an isotonic crystalloid solution; and
(c) a cryoprotectant agent;
wherein said formulation comprises a pH range of about 6.5-8.0.

2. The frozen formulation of claim 1, wherein the concentration of said apoptotic cells is between about 17-150×10⁶ cells/ml.

3. The frozen formulation of claim 1, wherein said mononuclear enriched cells comprise at least one cell type selected from monocytes, lymphocytes, T cells, B cells, and natural killer cells, wherein optionally, said at least one cell type comprises a single cell type selected from monocytes, lymphocytes, T cells, B cells, and natural killer cells.

4. (canceled)

5. The frozen formulation of claim 1, wherein said isotonic crystalloid solution comprises sodium chloride, potassium chloride, magnesium chloride, sodium acetate, or sodium gluconate, or any combination thereof.

6. The frozen formulation of claim 1, wherein said isotonic crystalloid solution comprises 140 mmol/L sodium, 5 mmol/L potassium, 1.5 mmol/L magnesium, 98 mmol/L chloride, 27 mmol/L acetate, and 23 mmol/L gluconate.

7. The frozen formulation of claim 1, wherein said cryoprotectant agent comprises DMSO.

8. The frozen formulation of claim 7, wherein the percent DMSO in said formulation is at a concentration of about 2.5%.

9. The frozen formulation of claim 1, wherein said pH is at about 7.4.

10. A method of preparing a frozen apoptotic cell formulation comprising steps:
(a) resuspending a fresh apoptotic mononuclear enriched population of cells in an isotonic crystalloid solution having a pH of about 6.5-8.0;
(b) mixing said resuspend apoptotic cells of step (a) with a cryoprotectant agent; and
(c) gradually freezing said mix of step (b) to a temperature of about-196° C.

11. The method of claim 10, wherein the concentration of said resuspended freshly prepared apoptotic cells of step (a) is between about 25-300×10\hat{ }⁶ cells/ml.

12. The method of claim 10, wherein said mononuclear enriched cells comprise at least one cell type selected from monocytes, lymphocytes, T cells, B cells, and natural killer cells, wherein optionally said at least one cell type comprises a single cell type selected from monocytes, lymphocytes, T cells, B cells, and natural killer cells.

13. (canceled)

14. The method of claim 10, wherein said isotonic crystalloid solution comprises sodium chloride, potassium chloride, magnesium chloride, sodium acetate, and sodium gluconate.

15. The method of claim 10, wherein said isotonic crystalloid solution comprises 140 mmol/L sodium, 5 mmol/L potassium, 1.5 mmol/L magnesium, 98 mmol/L chloride, 27 mmol/L acetate, and 23 mmol/L gluconate.

16. The method of claim 10, wherein said isotonic crystalloid solution comprises a pH of about 7.4.

17. The method of claim 10, wherein said mixing of step (b) is 1:1 (vol/vol) with said cryoprotectant agent, wherein optionally said cryoprotectant agent comprises DMSO.

18. (canceled)

19. The method of claim 17, wherein the final concentration of DMSO is about 2.5% DMSO.

20. The method of claim 10, further comprising a step of irradiation of irradiating said apoptotic cell population following said resuspension step (a) and prior to said mixing step (b).

21. The method of claim 10, wherein said frozen formulation comprises a concentration of about 20-100×10\hat{ }⁶ cells/ml apoptotic cells.

22. A method of treating a subject in need with a frozen apoptotic formulation of claim 1 comprising:
(a) thawing said frozen formulation; and
(b) administering said thawed formulation or a portion thereof to said subject in need;
thereby treating said subject.

23. The method of claim 22, further comprising a step of analyzing said thawed apoptotic formulation for Annexin V staining, wherein administration comprises administering an apoptotic population of cells comprising at least 35% Annexin V+ cells.
